SEQUENCE LISTING (34 sequences)

SEQ ID NO: 1 - DNA, 6100 bp, S. commune
cccgtccctcaaggccgttctttcgctggcgaccgacccggtgttcgcgagaacctgttgtttctgacgatcatcagccctttcttctcgtcgctctttagctctccctagaccgtcttttactctactcttcgacgcacgccatgtccggcccaggatatggcaggaatccattcgacaatcccccgcccaacagaggtccctatggccagcagccaggtttcccggggcccggccctcggccttacgactcggacgcggacatgagccagacctatggcagcacaaccaggctcgccggcagtgccggttacagcgacagaaacggtgcgcacgtcgctaccgtacttcctcgatcgtcgattcacataccatgcaggcagcttcgacggcgaccgctcctacgcgccctcaattgactcgcgcgccagcgtgcccagcatatcgcccttcgcagacccgggtatcggctctaatgagccgtatcccgcttggtcggtcgaacgccagattcccatgtccacggaggagattgaggacatcttcctcgacctcacccaaaagtttggcttccagcgcgactccatgcggaatacggtgcgtgaataagcagcccactcgaccgcgggaacagcacaattgacctgtcacccagttcgacttcatgatgcacctcctcgattcccgtgcctcgcgcatgacgcccaaccaagctctgctcacgcttcacgccgactacattggtggccagcatgccaattaccggaagtggtatttcgccgcacagctcaacctcgatgacgcggtcgggcaaaccaataaccccggtatccagcgcttgaagaccatcaagggcgctacgaagaccaagtcgctcgacagcgcactcaaccgctggcgcaacgcgatgaacaacatgagccagtacgatcgcctccggcaaattgcgctctacctcctctgctggggtgaagcaggcaacatccgtctggcgcccgagtgcttgtgcttcatcttcaagtgcgcggacgactactacagaagtcccgagtgtcagaaccggatggaccccgtgccggaagggctgtacctgcagacggtcatcaagccgctctatcgcttcctacgtgatcaggcgtacgaagtcgttgatgggaagcaagtgaagcgcgagaaggaccacgaccagattatcggttatgacgacgtcaaccagttattctggtatccggaaggtttggctaagatcgtcatgtcggacaacgtgcgtatgatcttatcggttaaaattcgtccgctcacatctttccagacacgacttgtagatgtacctccggcgcagcggttcatgaagttcgccaagatcgagtggaaccgcgtcttcttcaagacgtactttgagaagcgctctactgcccatctcctggtcaacttcaaccgtatatggatcctccacgtctcgatgtacttcttctacacggcattcaactctccacgagtctacgcgccgcacggcaaactcgacccctcccctgagatgacctggtccgcgactgcccttggaggcgctgtgtccaccatgatcatgatccttgccactatcgcggagtacacctacatccccacgacatggaacaatgcgtcgcacctcaccacgcggctcattttcctcctggtcatcctcgcgctcactgctggcccaacattctatatcgccatgatagacggacgcacggacatcggccaagtaccactcatcgtggccatagtgcagttcttcatctccgtcgtcgccaccctcgctttcgctaccatcccttctggtcgcatgttcggcgaccgtgtggctggcaagtcaagaaagcacatggcatcgcagacgttcacagcgtcgtacccgtccatgaagcggtcatctcgcgtagcgagtatcatgctgtggcttttggtctttggctgcaaatacgtcgagtcttacttcttcttgacgtcctccttctccagcccgatcgcggtcatggcgcgtacgaaggtacagggctgcaacgaccgtatcttcggcagccagctgtgcacgaatcaggtcccgttcgcgctggcaatcatgtacgtgatggacctggtactgttcttcctggacacgtacctgtggtacatcatctggctggtgatcttctcgatggtgcgcgcgttcaagcttggtatctcgatctggacgccctggagcgagatcttcacccgcatgccgaagcgtatttacgcaaagctgctggcgacggccgagatggaggtcaagtataagcccaaggtatgctgaattcaatctggtcaggtgaattcaccctcatattgtggtacaggtgctcgtctcacaaatctggaacgcggtcatcatctccatgtaccgggagcatctcttgtccatcgagcacgtccagcgcttgctttaccaccaggttgatggtcccgatggccgccgcaccctcagggcaccgccgttcttcaccagccagcgaactgcgaagccaggcctgttcttccctcctggtggcgaggctgagcgccgcatctcgttctttgcctcatcgctgacgaccgcgctcccggagcctctgccgatcgacgccatgcccaccttcaccgtgctcgttccccattactccgagaagattctgctcagtctgcgcgagattatccgcgaggaggaccagaacacccgcgttaccttactggagtacctcaagcagctccaccctgtcgaatgggacaatttcgtcaaggacaccaagatcttggcggaagagtcgggagacgtccaggacgagaagcgcgcgcgcacggacgacttgccgttctattgcatcgggttcaagacctcgtcaccagagtacaccctgcgtacgcgtatctgggcctcactgcgcgcacagacgctgtaccgcacggtctccggtatgatgaactactccaaggcgattaagctcctctatcgcgtcgagaacccggatgtcgttcatgccttcggtgggaacacggaacgtcttgaacgcgagcttgagcgcatgtctcgccgcaagttcaagttcgtcatctcgatgcagcggtactccaagttcaacaaggaggagcaggagaacgccgagttccttctgcgcgcgtacccggatttgcagatcgcgtacctcgatgaagagcccggtcccagcaagagcgacgaggttcggttgttttcgacactcatcgacggacactccgaggtggacgagaagacgggccgccgcaagcccaagttccgcatcgagctgcccggtaaccccatcctcggtgacgggaagtcggataaccagaaccacgccatcgtcttctaccgcggcgagtacattcaggtcattgacgctaaccaggacaattacctggaagagtgtctcaagatccgtaatgtcctgggcgagtttgaggaatactccgtgtcgagccagagcccgtacgcgcagtggggccacaaggagttcaacaagtgccccgtcgctatcctgggttcccgcgagtacatcttctcggagaacatcggtatcctcggtgacatcgctgccggcaaggaacagacgttcggtaccattacggcgcgtgcgcttgcgtggatcggcggcaagctgcattacggtcacccggatttcctcaatgcgacgttcatgacgacgcgtggtggcgtgtcaaaagcgcagaagggcttgcatcttaacgaggatatcttcgctggtatgaccgccgtgtcccgcggagggcgcatcaagcacatggagtactaccagtgcggcaaaggtcgtgatctcggattcggcacgatcttgaacttccagaccaagatcggtactggtatgggcgagcagctgctctcgcgcgagtactactatctgggcacgcaattgcctatcgaccggttcttgacgttctactacgcgcacgctggtttccatgtcaacaacatcctggtcatctactccatccaggtcttcatggtcacccgtaagtgcaggccctcatgaccgccgagcaagcagtctaacggatgtgcagtgctgtacctgggcacattgaacaagcagctgttcatctgcaaggtcaactccaatggccaggttcttagtggacaagctgggtgctacaacctcatcccggtcttcgagtggattcgccggagtatcatctccatcttcttggtgttcttcatcgccttcttgccgttgttcttgcaaggtatgttcacttctcatgtgccatttgtcaatcgctcactcgtacgacagagctttgcgaacgcggaacaggaaaggcgttgctgcgtctcgggaagcacttcctgtcactgtcgcccatcttcgaagtgttctccacccaaatctactcgcaggcgctcttgaacaacatgagtttcggtggtgcgcgctacatcgctacaggacgcggtttcgcgacgagtcggatacccttcaacatcctctactcgcgtttcgcgccgccgagcatctacatgggcatgcgtaatctgctgctcttgctgtacgcgacgatggccatttggatcccacacctgatctacttctggttctccgtcctctccctctgcatcgcgccattcatgttcaatccgcatcaattctcgtacgctgacttcatcatcgactaccgggagttcttgcgctggatgtcgcgcggtaactcgcggacgaaggcgagtagctggtacggatattgccgtctgtcgcgtaccgcgattactgggtacaagaagaagaaactgggacacccgtcggagaagctgtcgggcgatgtgccgcgtgcgccgtggaggaacgtcatcttctcggagatcctttggcccatcggcgcgtgcatcatcttcatcgtcgcgtacatgttcgtcaaatcgttccctgacgagcagggcaacgcgccgccgagcccgctggtccgcattctgctcatcgcggttggccctactgtgtggaacgcggcggtgctcatcacgctgttcttcctgtcgctcttcctgggcccgatgatggatggctgggtcaagttcggctcagtcatggcggcacttgcgcatggtctagcgctcataggcatgctcacgttcttcgagttcttcgtacgtccttcgcgttgttgtggtcgagtgctttgctaacaccgccttcagtggttcctcgagctctgggatgcctcgcacgccgtgctcggcgtcatcgccattattgccgttcagcgcgggatccagaagatcctcattgccgtcttcctgacgcgtgagtacaagcacgacgagacgaaccgcgcgtggtggacaggtaaatggtatggacgcgggctgggtacctcggccatgtcccagccggcgcgcgagttcatcgtgaagatcgtggagatgtcgctgtggacgtcggacttcctgcttgcgcacctgttgctcatcatcttgacggtgccgctactgctgccgttcttcaactcgatccattcgacgatgctttgtgagtgatttgtagtcgttggtcacggatgattgctgactcgcgtgcagtctggttgcgcccttcgaagcagattaggcaacctctgttctccactaagcagaagcggcaacggcgatggattgtaagttcctttgattgctctggctaccgaccttcgctcacctgtctcaggtcatgaagtataccgtggtatatctcgtggtggtggctttcctcgttgcgctcatcgctctgcgtacgttttctgtcgcgctcaccctctattttcactaacgtttcctccagccgcgctcttccgcgagagcatccacttcaactgcgagatctgccagagtatatagtcatataacgacgtctatcgtatcgccggacgagagccccgtcgcctacacactgacatggaattgctgtgtatacaatcgatcttctgaccgcgtcgggggcgttgccgtctttctactatcaacttgcttgtgtatcaacatttcttctctccaagcctacattgacatagagtaatagcccatgttcatacaacaatcgcatagcattgcatataccat

SEQ ID NO: 2 - protein, 1740 aa, S. commune
MSGPGYGRNPFDNPPPNRGPYGQQPGFPGPGPRPYDSDADMSQTYGSTTRLAGSAGYSDRNGSFDGDRSYAPSIDSRASVPSISPFADPGIGSNEPYPAWSVERQIPMSTEEIEDIFLDLTQKFGFQRDSMRNTFDFMMHLLDSRASRMTPNQALLTLHADYIGGQHANYRKWYFAAQLNLDDAVGQTNNPGIQRLKTIKGATKTKSLDSALNRWRNAMNNMSQYDRLRQIALYLLCWGEAGNIRLAPECLCFIFKCADDYYRSPECQNRMDPVPEGLYLQTVIKPLYRFLRDQAYEVVDGKQVKREKDHDQIIGYDDVNQLFWYPEGLAKIVMSDNTRLVDVPPAQRFMKFAKIEWNRVFFKTYFEKRSTAHLLVNFNRIWILHVSMYFFYTAFNSPRVYAPHGKLDPSPEMTWSATALGGAVSTMIMILATIAEYTYIPTTWNNASHLTTRLIFLLVILALTAGPTFYIAMIDGRTDIGQVPLIVAIVQFFISVVATLAFATIPSGRMFGDRVAGKSRKHMASQTFTASYPSMKRSSRVASIMLWLLVFGCKYVESYFFLTSSFSSPIAVMARTKVQGCNDRIFGSQLCTNQVPFALAIMYVMDLVLFFLDTYLWYIIWLVIFSMVRAFKLGISIWTPWSEIFTRMPKRIYAKLLATAEMEVKYKPKVLVSQIWNAVIISMYREHLLSIEHVQRLLYHQVDGPDGRRTLRAPPFFTSQRTAKPGLFFPPGGEAERRISFFASSLTTALPEPLPIDAMPTFTVLVPHYSEKILLSLREIIREEDQNTRVTLLEYLKQLHPVEWDNFVKDTKILAEESGDVQDEKRARTDDLPFYCIGFKTSSPEYTLRTRIWASLRAQTLYRTVSGMMNYSKAIKLLYRVENPDVVHAFGGNTERLERELERMSRRKFKFVISMQRYSKFNKEEQENAEFLLRAYPDLQIAYLDEEPGPSKSDEVRLFSTLIDGHSEVDEKTGRRKPKFRIELPGNPILGDGKSDNQNHAIVFYRGEYIQVIDANQDNYLEECLKIRNVLGEFEEYSVSSQSPYAQWGHKEFNKCPVAILGSREYIFSENIGILGDIAAGKEQTFGTITARALAWIGGKLHYGHPDFLNATFMTTRGGVSKAQKGLHLNEDIFAGMTAVSRGGRIKHMEYYQCGKGRDLGFGTILNFQTKIGTGMGEQLLSREYYYLGTQLPIDRFLTFYYAHAGFHVNNILVIYSIQVFMVTLLYLGTLNKQLFICKVNSNGQVLSGQAGCYNLIPVFEWIRRSIISIFLVFFIAFLPLFLQELCERGTGKALLRLGKHFLSLSPIFEVFSTQIYSQALLNNMSFGGARYIATGRGFATSRIPFNILYSRFAPPSIYMGMRNLLLLLYATMAIWIPHLIYFWFSVLSLCIAPFMFNPHQFSYADFIIDYREFLRWMSRGNSRTKASSWYGYCRLSRTAITGYKKKKLGHPSEKLSGDVPRAPWRNVIFSEILWPIGACIIFIVAYMFVKSFPDEQGNAPPSPLVRILLIAVGPTVWNAAVLITLFFLSLFLGPMMDGWVKFGSVMAALAHGLALIGMLTFFEFFWFLELWDASHAVLGVIAIIAVQRGIQKILIAVFLTREYKHDETNRAWWTGKWYGRGLGTSAMSQPAREFIVKIVEMSLWTSDFLLAHLLLIILTVPLLLPFFNSIHSTMLFWLRPSKQIRQPLFSTKQKRQRRWIVMKYTVVYLVVVAFLVALIALPALFRESIHFNCEICQSI

SEQ ID NO: 3 - DNA, 5770 bp, S. commune
ctgtccaaagaagagatcgaggacatcttcctcgatctgacgcagaagtttggctttcagcgggattccatgcggaacatggtacgtggcgtatgcccatgtgcggcgttctgaggcctaaacgttttccgccagttcgacttcaccatgcagctgcttgacagccgagcgtctcgtatgacccccaaccaggcgctcctcaccctccacgccgactacattggtggccagcatgcgaactaccggaagtggtacttcgcggcgcagctcgaccttgacgacgccgtgggacaaactcagaatccgggtctcaaccgcctcaagtccactcgcggatcgggcaagcgaccacgccatgaaaagtcgctgaacacggcattggagcgctggcggcaagccatgaacaacatgtcgcagtatgaccgcttacgccagatcgcgctctacctgctctgctggggcgaagcggcgcaagtgcgattcatgcccgagtgcttgtgcttcatcttcaagtgcgccgacgactattatcgttcgccggagtgccagaacaggatggagccggtaccggagggtctctacctgaggacggtcgtaaagccgctctacagatttgtccgggatcaaggctatgaggtggtggagggaaaattcgtacggcgggaacgggatcacgaccaaatcattggttacgatgacgtgaatcagctgttctggtacccggagggcattgcccgtatcgtcctgtcggacaaggtaagcacctctgtgcatcttctgtgacatacagggctaattgtcgagcagagtcgtctggtcgacctccctccagcacagcgcttcatgaagttcgaccgtatcgagtggaatcgcgtcttcttcaagacgttctacgagactcgatcctttacgcatcttttggtcgacttcaaccgtatctgggtcgtgcacatcgctctctacttcttctacaccgcatacaactcccccacgatctacgccatcaacggcaacactccgacgtctctggcttggagcgcgactgcgctcggcggtgcggtagcgacaggtatcatgatcctcgccacgatcgccgagttctcgcacatccccacgacatggaacaacacctcgcatctgactcgccgcctcgccttcctcctcgtcacgctcggcctcacatgtggtccgacgttctacgtcgcgattgcagagagcaacgggagcggcggctctttggccttgattctcggcatcgtccagttcttcatctccgtcgtagcgactgcgctcttcactatcatgccttctggtcgtatgttcggcgaccgcgtcgcaggcaagagtcgcaagtatctcgccagccagacgttcacggccagctacccgtcgttgcccaagcaccagcggttcgcatcactcctgatgtggttcctcatcttcgggtgcaagttgacggagagttacttcttcctgacgttgtccttccgcgaccctattcgcgtcatggtcggcatgaagatccagaactgcgaggacaagattttcggcagcggcctttgcaggaatcacgcagcattcaccctcacgatcatgtacatcatggacctcgtcttgttcttcctcgacaccttcctttggtatgtcatctggaactcggttttcagtatcgcacgctctttcgtactcggcctttcgatctggacaccatggagggacatcttccagcgtctgccgaagcgtatctacgcgaagcttctagcgaccggcgacatggaggtcaagtacaagcccaaggtgtgtgaatagctcgctgtaaggttcttgattctgactcattcgcaggtcttggtttcgcaaatctggaacgccatcatcatctccatgtaccgcgagcacttgctctctatcgagcacgttcaaaagctcctgtaccatcaagtggacactggcgaagccggcaagcggagtcttcgcgcgcctccgttcttcgtcgcgcagggcagcagcggtggctcgggcgagttcttcccgcctggtagcgaggctgagcgtcgtatctctttcttcgcgcagtctctatctacggagattcctcagcccatcccggttgacgccatgccgacgttcacagtgcttacgcctcactacagcgagaaggtgcgctttttcctgggcgcattcaacattagctgactgtcgtgcacagatccttctttcgctccgtgagattatccgcgaggaggaccagaacacccgcgtgacattgcttgagtatctcaagcagcttcacccggtcgagtgggagaacttcgtcaaggacaccaagattttggccgaggagtccgctatgttcaacggtccaagtcctttcggcaacgatgagaagggtcagtccaagatggacgatcttcctttctactgcatcggtttcaagagcgccgcgcccgagtacaccctccgcacccgtatctgggcgtccttgcgcgcgcagaccctctaccgcacggtctccggcatgatgaactatgcgaaggcgattaagctgctctaccgcgtcgagaaccccgaggtcgtgcagcagttcggcggtaacacggacaagctcgagcgcgagttggagcggatggcccggcggaagttcaagttcctggtgtccatgcagcgctactcgaagttcaacaaggaggagcacgagaacgccgagttcttgctccgcgcgtacccggacctgcagatcgcgtacctggaggaagagcctcctcgcaaggagggtggcgatccacgcatcttctctgccctcgtcgacggccacagcgacatcatcccggagaccggcaagcggcgccccaagttccgcatcgagctgcccggcaaccccattctcggtgacggcaagtcggacaaccagaaccacgccatcgtcttctaccgcggcgagtacctccagcttatcgacgccaaccaggacaactacctcgaggagtgcttgaagatccgtaacgtactcgccgagttcgaggagtacgacgtctctagccagagtccgtacgcgcagtggagtgtcaaggagttcaagcgctccccggtcgccatcgtcggtgcacgcgagtatatcttctcggagcacatcggtattctcggtgatttggcggctggcaaggaacagacgttcggtacgctcacggcacgcaacaacgccttccttggcggcaagctgcactacggtcacccggatttcctcaacgccctctacatgaacacgcgcggtggtgtctccaaggcgcagaagggtctccatctcaacgaggatatttacgccggtatgaacgcggtcggtcgcggtggacgcatcaagcatagcgaatactaccagtgcggcaagggtcgtgacctcggttttggcaccatcttgaacttccagaccaagatcggtacgggtatgggcgagcagatcctctcgcgcgagtactactacctcggaacccaattgcccatcgatcgcttcctcacgttctactacgcgcacccaggtttccagatcaacaacatgctggttatcctatccgtgcaggtcttcatcgttaccagtacgttgattgcatatcgttagcctgacagcgtctgacgaattcccagtggtcttcctcggtaccttgaagtcttcggtcacgatctgcaagtacacgtccagcggtcagtacatcggtggtcaatccggttgctacaacctcgtcccggtcttccagtggatcgagcgctgcatcatcagcatcttcttggtgttcatgatcgctttcatgccgctcttcctgcaaggtaagagctcgtcaacctgctcaagggccttgcgctgatcatcatctcagaactcgtcgagcgcggtacctggagtgccatctggcgtctgctcaagcagtttatgtcgctgtcgcctgtcttcgaggtgttctccacccagattcagacacactccgtgttgagcaacttgacgttcggtggtgcgcgttacatcgctaccggtcgtgggttcgccaccagtcgtatcagcttcagcatcttgttctcgcgtttcgcaggcccgagtatctacctcggcatgcgcacgctcattatgctgctctacgtgacgttgacgatctggacgccatgggtcatttacttctgggtttccattctctcgctctgcatcgcgccgttcttgttcaatccgcatcaattcgtcttctcggatttcctcatcgactacaggtacgtcggacgagcgctgttccgcgacgtaagctgaccggttatacagggaatacctccggtggatgtcgcgtggtaactcgcgctcgcacaacaactcctggattgggtactgccggttgtcccgcacgatgatcactgggtacaagaagaagaagctgggccacccgtcggagaagctttccggcgacgttcctcgtgcaggctggcgcgccgtcttattctcggagatcatcttcccggcatgcatggccatcctcttcatcatcgcgtacatgttcgtcaagtcgttccctctcgacggcaagcagcctccctccggcctcgttcgcatcgccgtcgtgtctatcggccccatcgtgtggaacgccgccatcctgttgacgctcttccttgtgtcgttgttcctcggccccatgctcgacccggtcttccccctcttcggttccgttatggccttcatcgcgcatttcctcggcacaatcggaatgattgggttcttcgagttcctggtatgtgcccatacctttcattcgtcttcaactatctaacagattcatagtggttcctcgagtcctgggaggcgtcgcatgccgtgctgggtctcatcgccgtcatctccatccagcgcgccattcacaaaattcttatcgccgttttcctcagtcgcgagttcaagcacgacgagacgaacagggcttggtggactggtcgctggtatggccgtggcctcggcacgcacgccatgtcgcagccggcgcgtgagttcgtcgtcaagatcatcgagttgtcgctctggagctcggatctcatactcggccacatcctgctgttcatgcttactccggctgtcctcatcccgtacttcgaccgtctgcacgccatgatgctctgtacgtcgtgtctcattgtttgtgttggtcatactcttaccctctcttagtctggctgcgcccctcaaagcaaatccgcgcgcctctgtactcaatcaagcagaagaggcaaagacgctggattgtcagtgttcagtgccttattctatcagctcttactgacgtcttcatagatcatgaagtacggtactgtatacgttaccgtcatcgcgatcttcgtcgcgctcatcgcgcttcgtgagtacccttgctatctttcgtacctgagcgtcgctgacccctttcccagccctcgtcttccgacacactctaaaggtcgagtgctccctttgcgacagcttgtaatatcggactcgtatatatctagacttctccgcaccatgtgtagctgacgcttgggtatacttcgcggtgccgagctaattgtcgacggacattctccatcgttgagtgcagcgacatcgggtggtttacgacacggacacttttcattgtaccctctacgaatgcaagaactctcttacgaccagtacctatgtgctaagccgtcgcctgttcaggatcatacatacatacgtttctagataccttacagttaggcctattcagggagagtctgcataaaa

SEQ ID NO: 4 - protein, 1622 aa, S. commune
MRNMFDFTMQLLDSRASRMTPNQALLTLHADYIGGQHANYRKWYFAAQLDLDDAVGQTQNPGLNRLKSTRGSGKRPRHEKSLNTALERWRQAMNNMSQYDRLRQIALYLLCWGEAAQVRFMPECLCFIFKCADDYYRSPECQNRMEPVPEGLYLRTVVKPLYRFVRDQGYEVVEGKFVRRERDHDQIIGYDDVNQLFWYPEGIARIVLSDKSRLVDLPPAQRFMKFDRIEWNRVFFKTFYETRSFTHLLVDFNRIWVVHIALYFFYTAYNSPTIYAINGNTPTSLAWSATALGGAVATGIMILATIAEFSHIPTTWNNTSHLTRRLAFLLVTLGLTCGPTFYVAIAESNGSGGSLALILGIVQFFISVVATALFTIMPSGRMFGDRVAGKSRKYLASQTFTASYPSLPKHQRFASLLMWFLIFGCKLTESYFFLTLSFRDPIRVMVGMKIQNCEDKIFGSGLCRNHAAFTLTIMYIMDLVLFFLDTFLWYVIWNSVFSIARSFVLGLSIWTPWRDIFQRLPKRIYAKLLATGDMEVKYKPKVLVSQIWNAIIISMYREHLLSIEHVQKLLYHQVDTGEAGKRSLRAPPFFVAQGSSGGSGEFFPPGSEAERRISFFAQSLSTEIPQPIPVDAMPTFTVLTPHYSEKILLSLREIIREEDQNTRVTLLEYLKQLHPVEWENFVKDTKILAEESAMFNGPSPFGNDEKGQSKMDDLPFYCIGFKSAAPEYTLRTRIWASLRAQTLYRTVSGMMNYAKAIKLLYRVENPEVVQQFGGNTDKLERELERMARRKFKFLVSMQRYSKFNKEEHENAEFLLRAYPDLQIAYLEEEPPRKEGGDPRIFSALVDGHSDIIPETGKRRPKFRIELPGNPILGDGKSDNQNHAIVFYRGEYLQLIDANQDNYLEECLKIRNVLAEFEEYDVSSQSPYAQWSVKEFKRSPVAIVGAREYIFSEHIGILGDLAAGKEQTFGTLTARNNAFLGGKLHYGHPDFLNALYMNTRGGVSKAQKGLHLNEDIYAGMNAVGRGGRIKHSEYYQCGKGRDLGFGTILNFQTKIGTGMGEQILSREYYYLGTQLPIDRFLTFYYAHPGFQINNMLVILSVQVFIVTMVFLGTLKSSVTICKYTSSGQYIGGQSGCYNLVPVFQWIERCIISIFLVFMIAFMPLFLQELVERGTWSAIWRLLKQFMSLSPVFEVFSTQIQTHSVLSNLTFGGARYIATGRGFATSRISFSILFSRFAGPSIYLGMRTLIMLLYVTLTIWTPWVIYFWVSILSLCIAPFLFNPHQFVFSDFLIDYREYLRWMSRGNSRSHNNSWIGYCRLSRTMITGYKKKKLGHPSEKLSGDVPRAGWRAVLFSEIIFPACMAILFIIAYMFVKSFPLDGKQPPSGLVRIAVVSIGPIVWNAAILLTLFLVSLFLGPMLDPVFPLFGSVMAFIAHFLGTIGMIGFFEFLWFLESWEASHAVLGLIAVISIQRAIHKILIAVFLSREFKHDETNRAWWTGRWYGRGLGTHAMSQPAREFVVKIIELSLWSSDLILGHILLFMLTPAVLIPYFDRLHAMMLFWLRPSKQIRAPLYSIKQKRQRRWIIMKYGTVYVTVIAIFVALIALPLVFRHTLKVECSLCDSL

SEQ ID NO: 5 - DNA, 5223 bp, S. commune
atgtccggcccaggatatggcaggaatccattcgacaatcccccgcccaacagaggtccctatggccagcagccaggtttcccggggcccggccctcggccttacgactcggacgcggacatgagccagacctatggcagcacaaccaggctcgccggcagtgccggttacagcgacagaaacggcagcttcgacggcgaccgctcctacgcgccctcaattgactcgcgcgccagcgtgcccagcatatcgcccttcgcagacccgggtatcggctctaatgagccgtatcccgcttggtcggtcgaacgccagattcccatgtccacggaggagattgaggacatcttcctcgacctcacccaaaagtttggcttccagcgcgactccatgcggaatacgttcgacttcatgatgcacctcctcgattcccgtgcctcgcgcatgacgcccaaccaagctctgctcacgcttcacgccgactacattggtggccagcatgccaattaccggaagtggtatttcgccgcacagctcaacctcgatgacgcggtcgggcaaaccaataaccccggtatccagcgcttgaagaccatcaagggcgctacgaagaccaagtcgctcgacagcgcactcaaccgctggcgcaacgcgatgaacaacatgagccagtacgatcgcctccggcaaattgcgctctacctcctctgctggggtgaagcaggcaacatccgtctggcgcccgagtgcttgtgcttcatcttcaagtgcgcggacgactactacagaagtcccgagtgtcagaaccggatggaccccgtgccggaagggctgtacctgcagacggtcatcaagccgctctatcgcttcctacgtgatcaggcgtacgaagtcgttgatgggaagcaagtgaagcgcgagaaggaccacgaccagattatcggttatgacgacgtcaaccagttattctggtatccggaaggtttggctaagatcgtcatgtcggacaacacacgacttgtagatgtacctccggcgcagcggttcatgaagttcgccaagatcgagtggaaccgcgtcttcttcaagacgtactttgagaagcgctctactgcccatctcctggtcaacttcaaccgtatatggatcctccacgtctcgatgtacttcttctacacggcattcaactctccacgagtctacgcgccgcacggcaaactcgacccctcccctgagatgacctggtccgcgactgcccttggaggcgctgtgtccaccatgatcatgatccttgccactatcgcggagtacacctacatccccacgacatggaacaatgcgtcgcacctcaccacgcggctcattttcctcctggtcatcctcgcgctcactgctggcccaacattctatatcgccatgatagacggacgcacggacatcggccaagtaccactcatcgtggccatagtgcagttcttcatctccgtcgtcgccaccctcgctttcgctaccatcccttctggtcgcatgttcggcgaccgtgtggctggcaagtcaagaaagcacatggcatcgcagacgttcacagcgtcgtacccgtccatgaagcggtcatctcgcgtagcgagtatcatgctgtggcttttggtctttggctgcaaatacgtcgagtcttacttcttcttgacgtcctccttctccagcccgatcgcggtcatggcgcgtacgaaggtacagggctgcaacgaccgtatcttcggcagccagctgtgcacgaatcaggtcccgttcgcgctggcaatcatgtacgtgatggacctggtactgttcttcctggacacgtacctgtggtacatcatctggctggtgatcttctcgatggtgcgcgcgttcaagcttggtatctcgatctggacgccctggagcgagatcttcacccgcatgccgaagcgtatttacgcaaagctgctggcgacggccgagatggaggtcaagtataagcccaaggtgctcgtctcacaaatctggaacgcggtcatcatctccatgtaccgggagcatctcttgtccatcgagcacgtccagcgcttgctttaccaccaggttgatggtcccgatggccgccgcaccctcagggcaccgccgttcttcaccagccagcgaactgcgaagccaggcctgttcttccctcctggtggcgaggctgagcgccgcatctcgttctttgcctcatcgctgacgaccgcgctcccggagcctctgccgatcgacgccatgcccaccttcaccgtgctcgttccccattactccgagaagattctgctcagtctgcgcgagattatccgcgaggaggaccagaacacccgcgttaccttactggagtacctcaagcagctccaccctgtcgaatgggacaatttcgtcaaggacaccaagatcttggcggaagagtcgggagacgtccaggacgagaagcgcgcgcgcacggacgacttgccgttctattgcatcgggttcaagacctcgtcaccagagtacaccctgcgtacgcgtatctgggcctcactgcgcgcacagacgctgtaccgcacggtctccggtatgatgaactactccaaggcgattaagctcctctatcgcgtcgagaacccggatgtcgttcatgccttcggtgggaacacggaacgtcttgaacgcgagcttgagcgcatgtctcgccgcaagttcaagttcgtcatctcgatgcagcggtactccaagttcaacaaggaggagcaggagaacgccgagttccttctgcgcgcgtacccggatttgcagatcgcgtacctcgatgaagagcccggtcccagcaagagcgacgaggttcggttgttttcgacactcatcgacggacactccgaggtggacgagaagacgggccgccgcaagcccaagttccgcatcgagctgcccggtaaccccatcctcggtgacgggaagtcggataaccagaaccacgccatcgtcttctaccgcggcgagtacattcaggtcattgacgctaaccaggacaattacctggaagagtgtctcaagatccgtaatgtcctgggcgagtttgaggaatactccgtgtcgagccagagcccgtacgcgcagtggggccacaaggagttcaacaagtgccccgtcgctatcctgggttcccgcgagtacatcttctcggagaacatcggtatcctcggtgacatcgctgccggcaaggaacagacgttcggtaccattacggcgcgtgcgcttgcgtggatcggcggcaagctgcattacggtcacccggatttcctcaatgcgacgttcatgacgacgcgtggtggcgtgtcaaaagcgcagaagggcttgcatcttaacgaggatatcttcgctggtatgaccgccgtgtcccgcggagggcgcatcaagcacatggagtactaccagtgcggcaaaggtcgtgatctcggattcggcacgatcttgaacttccagaccaagatcggtactggtatgggcgagcagctgctctcgcgcgagtactactatctgggcacgcaattgcctatcgaccggttcttgacgttctactacgcgcacgctggtttccatgtcaacaacatcctggtcatctactccatccaggtcttcatggtcaccctgctgtacctgggcacattgaacaagcagctgttcatctgcaaggtcaactccaatggccaggttcttagtggacaagctgggtgctacaacctcatcccggtcttcgagtggattcgccggagtatcatctccatcttcttggtgttcttcatcgccttcttgccgttgttcttgcaagagctttgcgaacgcggaacaggaaaggcgttgctgcgtctcgggaagcacttcctgtcactgtcgcccatcttcgaagtgttctccacccaaatctactcgcaggcgctcttgaacaacatgagtttcggtggtgcgcgctacatcgctacaggacgcggtttcgcgacgagtcggatacccttcaacatcctctactcgcgtttcgcgccgccgagcatctacatgggcatgcgtaatctgctgctcttgctgtacgcgacgatggccatttggatcccacacctgatctacttctggttctccgtcctctccctctgcatcgcgccattcatgttcaatccgcatcaattctcgtacgctgacttcatcatcgactaccgggagttcttgcgctggatgtcgcgcggtaactcgcggacgaaggcgagtagctggtacggatattgccgtctgtcgcgtaccgcgattactgggtacaagaagaagaaactgggacacccgtcggagaagctgtcgggcgatgtgccgcgtgcgccgtggaggaacgtcatcttctcggagatcctttggcccatcggcgcgtgcatcatcttcatcgtcgcgtacatgttcgtcaaatcgttccctgacgagcagggcaacgcgccgccgagcccgctggtccgcattctgctcatcgcggttggccctactgtgtggaacgcggcggtgctcatcacgctgttcttcctgtcgctcttcctgggcccgatgatggatggctgggtcaagttcggctcagtcatggcggcacttgcgcatggtctagcgctcataggcatgctcacgttcttcgagttcttctggttcctcgagctctgggatgcctcgcacgccgtgctcggcgtcatcgccattattgccgttcagcgcgggatccagaagatcctcattgccgtcttcctgacgcgtgagtacaagcacgacgagacgaaccgcgcgtggtggacaggtaaatggtatggacgcgggctgggtacctcggccatgtcccagccggcgcgcgagttcatcgtgaagatcgtggagatgtcgctgtggacgtcggacttcctgcttgcgcacctgttgctcatcatcttgacggtgccgctactgctgccgttcttcaactcgatccattcgacgatgcttttctggttgcgcccttcgaagcagattaggcaacctctgttctccactaagcagaagcggcaacggcgatggattgtcatgaagtataccgtggtatatctcgtggtggtggctttcctcgttgcgctcatcgctctgcccgcgctcttccgcgagagcatccacttcaactgcgagatctgccagagtatatag

SEQ ID NO: 6 - protein, 1740 aa, S. commune; identical to SEQ ID NO: 2

SEQ ID NO: 7 - DNA, 4869 bp, S. commune
atgcggaacatgttcgacttcaccatgcagctgcttgacagccgagcgtctcgtatgacccccaaccaggcgctcctcaccctccacgccgactacattggtggccagcatgcgaactaccggaagtggtacttcgcggcgcagctcgaccttgacgacgccgtgggacaaactcagaatccgggtctcaaccgcctcaagtccactcgcggatcgggcaagcgaccacgccatgaaaagtcgctgaacacggcattggagcgctggcggcaagccatgaacaacatgtcgcagtatgaccgcttacgccagatcgcgctctacctgctctgctggggcgaagcggcgcaagtgcgattcatgcccgagtgcttgtgcttcatcttcaagtgcgccgacgactattatcgttcgccggagtgccagaacaggatggagccggtaccggagggtctctacctgaggacggtcgtaaagccgctctacagatttgtccgggatcaaggctatgaggtggtggagggaaaattcgtacggcgggaacgggatcacgaccaaatcattggttacgatgacgtgaatcagctgttctggtacccggagggcattgcccgtatcgtcctgtcggacaagagtcgtctggtcgacctccctccagcacagcgcttcatgaagttcgaccgtatcgagtggaatcgcgtcttcttcaagacgttctacgagactcgatcctttacgcatcttttggtcgacttcaaccgtatctgggtcgtgcacatcgctctctacttcttctacaccgcatacaactcccccacgatctacgccatcaacggcaacactccgacgtctctggcttggagcgcgactgcgctcggcggtgcggtagcgacaggtatcatgatcctcgccacgatcgccgagttctcgcacatccccacgacatggaacaacacctcgcatctgactcgccgcctcgccttcctcctcgtcacgctcggcctcacatgtggtccgacgttctacgtcgcgattgcagagagcaacgggagcggcggctctttggccttgattctcggcatcgtccagttcttcatctccgtcgtagcgactgcgctcttcactatcatgccttctggtcgtatgttcggcgaccgcgtcgcaggcaagagtcgcaagtatctcgccagccagacgttcacggccagctacccgtcgttgcccaagcaccagcggttcgcatcactcctgatgtggttcctcatcttcgggtgcaagttgacggagagttacttcttcctgacgttgtccttccgcgaccctattcgcgtcatggtcggcatgaagatccagaactgcgaggacaagattttcggcagcggcctttgcaggaatcacgcagcattcaccctcacgatcatgtacatcatggacctcgtcttgttcttcctcgacaccttcctttggtatgtcatctggaactcggttttcagtatcgcacgctctttcgtactcggcctttcgatctggacaccatggagggacatcttccagcgtctgccgaagcgtatctacgcgaagcttctagcgaccggcgacatggaggtcaagtacaagcccaaggtcttggtttcgcaaatctggaacgccatcatcatctccatgtaccgcgagcacttgctctctatcgagcacgttcaaaagctcctgtaccatcaagtggacactggcgaagccggcaagcggagtcttcgcgcgcctccgttcttcgtcgcgcagggcagcagcggtggctcgggcgagttcttcccgcctggtagcgaggctgagcgtcgtatctctttcttcgcgcagtctctatctacggagattcctcagcccatcccggttgacgccatgccgacgttcacagtgcttacgcctcactacagcgagaagatccttctttcgctccgtgagattatccgcgaggaggaccagaacacccgcgtgacattgcttgagtatctcaagcagcttcacccggtcgagtgggagaacttcgtcaaggacaccaagattttggccgaggagtccgctatgttcaacggtccaagtcctttcggcaacgatgagaagggtcagtccaagatggacgatcttcctttctactgcatcggtttcaagagcgccgcgcccgagtacaccctccgcacccgtatctgggcgtccttgcgcgcgcagaccctctaccgcacggtctccggcatgatgaactatgcgaaggcgattaagctgctctaccgcgtcgagaaccccgaggtcgtgcagcagttcggcggtaacacggacaagctcgagcgcgagttggagcggatggcccggcggaagttcaagttcctggtgtccatgcagcgctactcgaagttcaacaaggaggagcacgagaacgccgagttcttgctccgcgcgtacccggacctgcagatcgcgtacctggaggaagagcctcctcgcaaggagggtggcgatccacgcatcttctctgccctcgtcgacggccacagcgacatcatcccggagaccggcaagcggcgccccaagttccgcatcgagctgcccggcaaccccattctcggtgacggcaagtcggacaaccagaaccacgccatcgtcttctaccgcggcgagtacctccagcttatcgacgccaaccaggacaactacctcgaggagtgcttgaagatccgtaacgtactcgccgagttcgaggagtacgacgtctctagccagagtccgtacgcgcagtggagtgtcaaggagttcaagcgctccccggtcgccatcgtcggtgcacgcgagtatatcttctcggagcacatcggtattctcggtgatttggcggctggcaaggaacagacgttcggtacgctcacggcacgcaacaacgccttccttggcggcaagctgcactacggtcacccggatttcctcaacgccctctacatgaacacgcgcggtggtgtctccaaggcgcagaagggtctccatctcaacgaggatatttacgccggtatgaacgcggtcggtcgcggtggacgcatcaagcatagcgaatactaccagtgcggcaagggtcgtgacctcggttttggcaccatcttgaacttccagaccaagatcggtacgggtatgggcgagcagatcctctcgcgcgagtactactacctcggaacccaattgcccatcgatcgcttcctcacgttctactacgcgcacccaggtttccagatcaacaacatgctggttatcctatccgtgcaggtcttcatcgttaccatggtcttcctcggtaccttgaagtcttcggtcacgatctgcaagtacacgtccagcggtcagtacatcggtggtcaatccggttgctacaacctcgtcccggtcttccagtggatcgagcgctgcatcatcagcatcttcttggtgttcatgatcgctttcatgccgctcttcctgcaagaactcgtcgagcgcggtacctggagtgccatctggcgtctgctcaagcagtttatgtcgctgtcgcctgtcttcgaggtgttctccacccagattcagacacactccgtgttgagcaacttgacgttcggtggtgcgcgttacatcgctaccggtcgtgggttcgccaccagtcgtatcagcttcagcatcttgttctcgcgtttcgcaggcccgagtatctacctcggcatgcgcacgctcattatgctgctctacgtgacgttgacgatctggacgccatgggtcatttacttctgggtttccattctctcgctctgcatcgcgccgttcttgttcaatccgcatcaattcgtcttctcggatttcctcatcgactacagggaatacctccggtggatgtcgcgtggtaactcgcgctcgcacaacaactcctggattgggtactgccggttgtcccgcacgatgatcactgggtacaagaagaagaagctgggccacccgtcggagaagctttccggcgacgttcctcgtgcaggctggcgcgccgtcttattctcggagatcatcttcccggcatgcatggccatcctcttcatcatcgcgtacatgttcgtcaagtcgttccctctcgacggcaagcagcctccctccggcctcgttcgcatcgccgtcgtgtctatcggccccatcgtgtggaacgccgccatcctgttgacgctcttccttgtgtcgttgttcctcggccccatgctcgacccggtcttccccctcttcggttccgttatggccttcatcgcgcatttcctcggcacaatcggaatgattgggttcttcgagttcctgtggttcctcgagtcctgggaggcgtcgcatgccgtgctgggtctcatcgccgtcatctccatccagcgcgccattcacaaaattcttatcgccgttttcctcagtcgcgagttcaagcacgacgagacgaacagggcttggtggactggtcgctggtatggccgtggcctcggcacgcacgccatgtcgcagccggcgcgtgagttcgtcgtcaagatcatcgagttgtcgctctggagctcggatctcatactcggccacatcctgctgttcatgcttactccggctgtcctcatcccgtacttcgaccgtctgcacgccatgatgctcttctggctgcgcccctcaaagcaaatccgcgcgcctctgtactcaatcaagcagaagaggcaaagacgctggattatcatgaagtacggtactgtatacgttaccgtcatcgcgatcttcgtcgcgctcatcgcgcttcccctcgtcttccgacacactctaaaggtcgagtgctccctttgcgacagcttgtaa

SEQ ID NO: 8 - protein, 1622 aa, S. commune; identical to SEQ ID NO: 4

SEQ ID NO: 9 - DNA, 6098 bp, S. commune
cccgtccctcaaggccgttctttcgctggcgaccgacccggtgttcgcgagaacctgttgtttctgacgatcatcaaccctttcttctcgtcgctctttagctctccctagaccgtcttttactctactcttcgacgcacgccatgtccggtccaggatatggcaggaatccattcgacaatcccccgcccaacagaggtccctatggccagcagccaggtttcccggggcccggccctcggccttacgactcggacgcggacatgagccagacctatggcagcacaaccaggctcgccggcagtgccggttacagcgacagaaacggtgcgaacgtcgctaccgtacttcctcgatcgtcgactcacatatcacgcaggcagcttcgacggcgaccgctcctacgcgccctcaattgactcgcgcgccagcgtgcccagcatatcgcccttcgcagacccgggtatcggctctaatgagccgtatcccgcttggtcggtcgaacgccagatccccatgtccacggaggagattgaggatatcttcctcgacctcacccaaaagtttggcttccagcgcgactccatgcggaatacggtgcgtgaataagcagcccactcgaccgcgggaacagctcaattgacctgtcacccagttcgacttcatgatgcacctccttgattcccgtgcctcgcgcatgacgcccaaccaagctctgctcacgcttcacgccgactacattggtggccagcacgccaactataggaagtggtatttcgccgctcagctcaacctcgatgacgcggtcgggcaaaccaataaccccggtatccagcgcttgaagaccatcaagggcgctacgaagaccaagtcgctcgacagcgcactcaaccgctggcgcaatgcgatgaacaacatgagccagtacgatcgcctccggcaaattgcgctctatctcctctgctggggagaagcaggcaacatccgtctggcgcccgagtgcttgtgcttcatcttcaagtgcgcggacgactactacagaagtcccgagtgtcagaaccggatggaccccgtgccggaagggctgtacctccagacggtcatcaagccgctctatcgcttcctacgtgatcaggcgtacgaagtcgttgatgggaagcaagtgaagcgcgagaaggaccacgaccagattatcggttatgacgacgtcaaccagttattctggtatccggaaggtttggctaagatcgtcatgtcggacaacgtgcgtatgatcttatcggttacaattcgtccgctcacatctttccagacacgacttgtagatgtacctccggcgcagcggttcatgaagttcgccaagatcgagtggaaccgcgtcttcttcaagacgtactttgagaagcgctctactgcccatctcctggtcaacttcaaccgtatatggatcctccacgtctcgatgtacttcttctacacggcattcaactctccacgagtctacgcgccgcacggcaaactcgacccctcccctgagatgacctggtccgcgactgcccttggaggcgctgtgtccaccatgatcatgatccttgccactatcgcggagtacacctacatccccacgacatggaacaatgcgtcgcacctcaccacgcggctcattttcctcctggtcatcctcgcgctcactgctggaccaacattctatatcgccatgatagacggacgcacggacatcggccaagtaccactcatcgtggccatagtgcagttcttcatctccgtcgtcgccaccctcgctttcgctaccatcccttctggtcgcatgttcggcgaccgtgtggctggcaagtcaagaaagcacatggcatcgcagacgttcacagcgtcgtacccgtccatgaagcggtcatctcgcgtagcgagtatcatgctgtggcttttggtctttggctgcaaatacgtcgagtcttacttcttcttgacgtcctccttctccagcccgatcgcggtcatggcgcgtacgaaggtacagggctgcaacgaccgtatcttcggcagccagctgtgcacgaatcaggtcccgttcgcgctggcaatcatgtacgtgatggacctggtactgttcttcctggacacgtacctgtggtacatcatctggctggtgatcttctcgatggtgcgcgcgttcaagcttggtatctcgatctggacgccctggagcgagatcttcacccgcatgccgaagcgtatctacgcgaagctgctggcgacggccgagatggaggtcaagtataagcccaaggtatgctgaatgcaatctggtcaggtgaattcaccctcatattgttgtgcaggtgctcgtctcgcaaatctggaacgcggtcatcatctccatgtaccgggagcatctcttgtccatcgagcacgtccagcgcctgctataccaccaggttgatggtccagacggtcgccgcaccctcagggcaccgccgttcttcaccagccagcgaactgcgaagccaggcctgttcttccctcctggtggcgaggctgagcgccgtatctcgttctttgcctcatcgctgacgaccgcgctccctgagcctctgccgatcgacgccatgcccaccttcaccgtgctcgttccccattactcggagaagattctgctcagtctgcgcgagattattcgcgaggaggaccagaacacccgcgtcaccttgctggagtacctcaagcagctccaccctgtcgaatgggacaacttcgtcaaggacaccaagatcttggcggaagagtcgggcgacgtccaggacgagaagcgcgcgcgcacggacgacttgccgttctactgcatcgggttcaagacctcgtcaccagagtacaccctgcgtacgcgtatctgggcttcactgcgcgcacagacgctgtaccgcacggtctccggtatgatgaactactccaaggcgatcaagctcctctatcgcgtcgagaacccggatgtcgttcatgccttcggtgggaacacggaacgtcttgaacgcgagcttgagcgcatgtctcgccgcaagttcaagttcgtcatctcgatgcagcggtactctaagttcaacaaggaggagcaagagaacgccgaattccttctgcgcgcgtacccggatttgcagatcgcgtacctcgatgaagagcccggtcccagcaagagcgacgaggttcggttgttttcgacactcatcgatggacactccgaggtggatgagaagaccggccgccgcaagcccaagttccgcattgagctgcccggtaaccccatcctcggtgacgggaagtcggataaccagaaccacgccattgtcttctaccgcggcgagtacatccaggtcatcgacgctaaccaggacaattacctggaagagtgtctcaagatccgtaacgtcctgggcgagtttgaggaatactccgtgtcgagccagagcccgtacgcacagtggggccacaaggagttcaacaagtgccccgtcgctatcctgggttctcgcgagtacatcttctcggagaacatcggtatcctcggtgacatcgccgccggcaaggaacagacgttcggtaccattacggcgcgtgcgcttgcgtggatcggcggcaagctgcattacggtcacccggatttcctcaatgcgacgttcatgacgacgcgtggtggcgtgtcaaaagcgcagaagggcttgcatctcaacgaggatatcttcgctggtatgaccgccgtgtcccgcggagggcgcatcaagcacatggagtactaccagtgcggcaaaggtcgtgatctcggtttcggcacgatcttgaacttccagacgaagatcggtactggtatgggcgagcagctcctctcgcgcgagtactactacctgggcacgcaattgcctatcgaccggttcttgacgttctactacgcgcacgctggtttccacgtcaacaacatcctggtcatctactccatccaggtcttcatggtcacctgtaagtgcaggcgctcatgaccgccgagaacgtagtctgacggatgtgcagtgctgtacctgggcacattgaacaagcagctgttcatctgcaaggtcaactccaatggccaggttcttagtggacaagctgggtgctacaacctcatcccggtcttcgagtggattcgccggagtatcatctccatcttcttggtgttcttcatcgccttcttgcctctattcttgcaaggtatgttcactttccatgtgtcatccgttagccgctcaccatacgacagagctgtgcgagcgcggaacgggaaaggcgttgctgcgtctcgggaagcacttcttgtcactgtcgcccattttcgaagtgttctccacccagatttactcgcaggcgctcttgaacaacatgagcttcggtggtgcgcgctacatcgccacaggtcgtggtttcgcgactagtcgcatacccttcaacatcctctactcgcgtttcgcgccgccaagcatctacatgggcatgcgtaacctgctgctcctgctgtacgcgacgatggccatttggatcccgcacctgatctacttctggttctccgtcctctccctctgcatcgcgccattcatgttcaatccgcatcaattctcgtacgccgacttcatcatcgactaccgggagttcttgcgctggatgtcgcgcggtaactcgcgaacgaaggcgagcagctggtacggatactgccgtctgtcgcgtaccgcgattactgggtacaagaagaagaagctgggacacccgtcggagaagctgtcgggcgacgtaccgcgtgcgccgtggaggaacgttatcttctcggagatcctgtggcccatcggcgcgtgcatcatcttcatcgtcgcgtacatgttcgtcaagtcgttccccgacgagcagggcaacgcgccgccgagcccgctggtccggattctgctcatcgcggttggccctactgtgtggaacgcggcggtgctcataacgctgttcttcctgtcgctcttcctgggcccgatgatggatggctgggtcaagttcggctcggtcatggcggcccttgcgcatggcctggcgcttataggcatgctcacgttctttgagttcttcgtacgtccttcgcgttgtgtcgtcaagtgctctgctaacgccgtcttcagtggttccttgagctctgggatgcctcgcacgccgtgctcggcgtcatcgctatcattgccgttcagcgcgggatccagaagatcctcattgccgtcttcctgacgcgtgagtacaagcacgacgagacgaaccgcgcgtggtggacaggtaaatggtatggacgcgggctgggtacctcggccatgtcccagccggcgcgcgagttcatcgtgaagatcgtggagatgtcgttgtggacgtcggacttcctgcttgcgcacctgttgctcatcatcttgacggtgccgctactgctgccgttcttcaactcaattcattcgacgatgctttgtgagtggtttgtagtcgttggtcatggatgatttctgactcgcgtgcagtctggttgcgcccttcgaagcagattaggcaacctctgttctccaccaagcagaagcggcaacggcgatggattgtgagttcctttgattgctctgggtaccgaccttcgctcacctttcttaggtcatgaagtataccgtggtatatctcgtggtggtggctttcctcgtcgcgctcatcgctctgcgtacgttttccctcgcgctcaccctgtattttcactaacgtttcctccagccgccctcttccgcgagagcatccacttcaactgcgagatctgccagagtatatagtcatataacgacgtctatcgtatcgccggacgagagccccgtcgcctacacactgacatggaatcgctgtgtatacaatcgatcttctgaccgcgtcgggggcgttgccgtctttctactatcaatttgcttgtgtatcaacatttcttctctccaagcctacattgacatagagtaatagcccatgttcatacaacaatcgcatagcattgcatataccat

SEQ ID NO: 10 - protein, 1726 aa, S. commune
MSGPGYGRNPFDNPPPNRGPYGQQPGFPGPGPRPYDSDADMSQTYGSTTRLAGSAGYSDRNGSFDGDRSYAPSIDSRASVPSISPFADPGIGSNEPYPAWSVERQIPMSTEEIEDIFLDLTQKFGFQRDSMRNTFDFMMHLLDSRASRMTPNQALLTLHADYIGGQHANYRKWYFAAQLNLDDAVGQTNNPGIQRLKTIKGATKTKSLDSALNRWRNAMNNMSQYDRLRQIALYLLCWGEAGNIRLAPECLCFIFKCADDYYRSPECQNRMDPVPEGLYLQTVIKPLYRFLRDQAYEVVDGKQVKREKDHDQIIGYDDVNQLFWYPEGLAKIVMSDNTRLVDVPPAQRFMKFAKIEWNRVFFKTYFEKRSTAHLLVNFNRIWILHVSMYFFYTAFNSPRVYAPHGKLDPSPEMTWSATALGGAVSTMIMILATIAEYTYIPTTWNNASHLTTRLIFLLVILALTAGPTFYIAMIDGRTDIGQVPLIVAIVQFFISVVATLAFATIPSGRMFGDRVAGKSRKHMASQTFTASYPSMKRSSRVASIMLWLLVFGCKYVESYFFLTSSFSSPIAVMARTKVQGCNDRIFGSQLCTNQVPFALAIMYVMDLVLFFLDTYLWYIIWLVIFSMVRAFKLGISIWTPWSEIFTRMPKRIYAKLLATAEMEVKYKPKVLVSQIWNAVIISMYREHLLSIEHVQRLLYHQVDGPDGRRTLRAPPFFTSQRTAKPGLFFPPGGEAERRISFFASSLTTALPEPLPIDAMPTFTVLVPHYSEKILLSLREIIREEDQNTRVTLLEYLKQLHPVEWDNFVKDTKILAEESGDVQDEKRARTDDLPFYCIGFKTSSPEYTLRTRIWASLRAQTLYRTVSGMMNYSKAIKLLYRVENPDVVHAFGGNTERLERELERMSRRKFKFVISMQRYSKFNKEEQENAEFLLRAYPDLQIAYLDEEPGPSKSDEVRLFSTLIDGHSEVDEKTGRRKPKFRIELPGNPILGDGKSDNQNHAIVFYRGEYIQVIDANQDNYLEECLKIRNVLGEFEEYSVSSQSPYAQWGHKEFNKCPVAILGSREYIFSENIGILGDIAAGKEQTFGTITARALAWIGGKLHYGHPDFLNATFMTTRGGVSKAQKGLHLNEDIFAGMTAVSRGGRIKHMEYYQCGKGRDLGFGTILNFQTKIGTGMGEQLLSREYYYLGTQLPIDRFLTFYYAHAGFHVNNILVIYSIQVFMVTLLYLGTLNKQLFICKVNSNGQVLSGQAGCYNLIPVFEWIRRSIISIFLVFFIAFLPLFLQELCERGTGKALLRLGKHFLSLSPIFEVFSTQIYSQALLNNMSFGGARYIATGRGFATSRIPFNILYSRFAPPSIYMGMRNLLLLLYATMAIWIPHLIYFWFSVLSLCIAPFMFNPHQFSYADFIIDYREFLRWMSRGNSRTKASSWYGYCRLSRTAITGYKKKKLGHPSEKLSGDVPRAPWRNVIFSEILWPIGACIIFIVAYMFVKSFPDEQGNAPPSPLVRILLIAVGPTVWNAAVLITLFFLSLFLGPMMDGWVKFGSVMAALAHGLALIGMLTFFEFFWFLELWDASHAVLGVIAIIAVQRGIQKILIAVFLTRKWYGRGLGTSAMSQPAREFIVKIVEMSLWTSDFLLAHLLLIILTVPLLLPFFNSIHSTMLFWLRPSKQIRQPLFSTKQKRQRRWIVMKYTVVYLVVVAFLVALIALPALFRESIHFNCEICQSI

SEQ ID NO: 11 - DNA, 5771 bp, S. commune
ctgtccaaggaggagatcgaggacatcttcctcgatttgacgcagaagtttggctttcagcgggattccatgcggaatatggtacgtggcgtgtgcccatgtgcggcgttctgaggcctaacgttttccgccagttcgacttcaccatgcagctgcttgacagccgagcgtctcgtatgacccccaaccaggcgctcctcaccctccacgccgactacattggtggccagcatgcgaactaccggaagtggtacttcgcggcgcagctcgaccttgacgacgccgtgggacaaactcagaatccgggtctcaaccgcctcaagtccactcgcggatcgggcaagcgaccacgccatgaaaagtcgctgaacacggcattggagcgctggcggcaagccatgaacaacatgtcgcagtatgaccgcttacgccagatcgcgctctacctgctctgctggggcgaagcggcgcaagtgcgattcatgcccgagtgcttgtgcttcatcttcaagtgcgccgacgactactatcgttcgccggagtgccagaacaggatggagccggtaccggagggtctctacctgaggacggtcgtaaagccgctctacagatttgtccgggatcaaggctatgaggtggtggagggaaaattcgtacggcgggaacgggatcacgaccaaatcattggttacgatgacgtgaatcagctgttctggtacccggagggaattgcccgtatcgtcctgtcggacaaggtaagcacctctgtgcatcttctgtgacatacagggctaattgtcgagcagagtcgtctagtcgacctccccccagcacagcgcttcatgaagttcgaccgtatcgagtggaatcgcgtcttcttcaagacgttttacgagactcgatccttcacgcatcttttggtcgacttcaaccgtatctgggtcgtgcacatcgctctctacttcttctacactgcatacaactcccccacgatctacgccatcaacggcaacacaccgacgtctctggcttggagcgcgactgcgctcggcggtgcggtagcgacaggtatcatgatcctcgccacgatcgccgagttctcgcacatccccacgacatggaacaacacctcgcatctgactcgccgcctcgccttcctcctcgtcacgctcggcctcacatgtggtccgacgttctacgtcgcgattgcagagagcaacgggagcggcggctctttggccttgattctcggtatcgtccagttcttcatctccgtcgtggcaactgcgctcttcactatcatgccttctggtcgtatgttcggcgaccgtgtcgcaggcaagagtcgcaagtatctcgccagccagacgttcacggccagctacccgtcgttgcccaagcaccagcggttcgcctcactcctgatgtggttcctcatcttcgggtgcaagttgacggagagttacttctttctgacgctgtccttccgcgaccctatccgcgtcatggtcggcatgaagatccagaactgcgaggacaagattttcggcagcggcctttgcaggaatcacgcagcattcaccctcacgatcatgtacatcatggacctcgtcttgttcttcctcgacaccttcctttggtatgtcatctggaactcggttttcagtatcgcacgctctttcgtactcggcctttcgatctggacaccgtggagagacatcttccagcgtctgccgaagcggatctacgcgaagcttctggcgactggcgacatggaggtcaagtacaagcccaaggtatgcgttgagctcgccgtaaatccacttaaggctaacacgttcgcaggtcttggtctcgcaaatctggaacgccatcatcatctccatgtaccgcgagcacttgctctctattgagcacgtccagaagctcctgtaccaccaagtggacactggcgaagccggcaagcggagtcttcgcgcgcctccgttcttcgtcgcgcagggcagcagcggtggctcgggcgagttcttcccgcctggcagcgaggccgagcgtcgtatctctttcttcgcgcagtcgctttctacggagattcctcagcccatcccggtcgacgccatgccgacgttcacggtgcttacgcctcactacagcgagaaggtacatgctccccttgtagccatatgacatcagctgactgtcgtgcacagatccttctctctctccgtgaaattatccgcgaggaggaccagaacactcgcgttacgttgctcgagtacctgaagcagctgcatccggtcgagtgggagaatttcgtcaaggacactaaaattttggccgaggagtccgctatgtttaacggtccgagtcctttcggcaacgacgagaagggtcagtccaagatggacgatctaccgttctactgcatcggtttcaagagcgccgcgcccgagtacaccctccgcacccgtatctgggcgtccctgcgcgcgcagacgctgtaccgcacggtctccggcatgatgaactatgcgaaggcgatcaagctgctctaccgcgttgagaacccggaggtcgtacaacagttcggcggcaacacggacaagctcgagcgcgagttggagcggatggcgcgacggaagttcaagttcctcgtgtccatgcagcgctactcgaagttcaacaaggaggagcacgagaacgccgagttcttgctccgcgcgtacccggacttgcagatcgcgtacctcgaggaagagccccctcgcaaggagggcggcgatccacgcatcttctctgccctcgtcgacggccacagcgacatcatcccggagaccggcaagcggcgccccaagttccgtatcgagctgcccggtaaccccattctcggtgacggtaaatccgacaatcagaaccacgctatcgtcttctaccgcggcgagtacctccagcttatcgacgccaaccaggacaactacctcgaggagtgcttgaagatccgtaacgtgctcgccgagtttgaggagtacgacgtctccagccagagcccgtacgcgcagtggagtgtcaaggagttcaagcgctctccggtcgccatcgtcggtgcacgcgagtacatcttctcagagcacatcggtatcctcggtgatctggcggctggcaaggaacagacgttcggtacgctcacggcacgcaacaacgccttccttggcggcaagctgcactacggtcaccccgatttcctcaacgccctctacatgaacacgcgcggtggtgtctccaaggcgcagaagggtctccatctcaacgaggatatctacgccggtatgaacgcggtcggtcgcggtggacgcattaagcacagcgagtactatcagtgcggcaagggtcgtgacctcggtttcggcaccatcttgaacttccagaccaagatcggtacgggtatgggcgagcagatcctctcgcgcgagtactactatctcggaacacaactgcccatcgatcgcttcctcacgttctactacgcgcacccgggtttccagatcaacaacatgctggtcatcctctccgtgcaggtcttcatcgttaccagtacgttcaatgcatattgttagcctgacaacgtctgacgaatttccagtggtcttcctcggtaccttgaagtcttcggtcacgatctgcaagtacacgtccagcggtcagtacatcggtggtcaatccggttgctacaacctcgtcccggtcttccagtggatcgagcgctgcatcatcagcatcttcttggtgttcatgatcgctttcatgccgctcttcctgcaaggtaagagcttgtcaacctgctcaaggggcttgcgctgatcatcatctcagaactcgtcgagcgcggtacctggagtgccatctggcgtctgctcaagcagtttatgtcgctgtcgcctgtcttcgaggtgttctccacccagattcagacgcactccgtgttgagcaacttgacgttcggtggtgcgcgttacatcgctaccggtcgtgggttcgccaccagtcgtatcagcttcagcatcttgttctcgcgtttcgcaggcccgagtatctacctcggcatgcgcacgctcattatgctgctctacgtgacgttgacgatctggacgccatgggtcatttacttctgggtttccattctctcgctctgcatcgcgccgttcttgttcaacccgcatcaattcgtattctcggacttcctcatcgactacaggtacgtcggacgagcgctgttccgcgacgtaagctgaccggttatacagggaatacctgcggtggatgtcgcgtggcaactcgcgctcgcacaacaactcctggattgggtactgccggttgtcccgcacgatgatcactgggtacaagaagaagaagctgggccacccgtcggagaagctttccggcgacgttcctcgtgcaggctggcgcgccgtcttgttctcggagatcatcttcccggcgtgcatggccatcctcttcatcatcgcgtacatgttcgtcaagtcgttccctctcgacggcaagcagcctccctccggcctcgttcgcatcgccgtcgtgtctatcggccccatcgtgtggaacgccgccatcctgttgacgctcttccttgtgtcgttgttcctcggccccatgctcgacccggtcttccccctcttcggttccgttatggccttcatcgcgcatttccttggcacaatcggaatgattgggttcttcgagttcctggtatgtgcccatacctttcattcgacttcaactatctaacagattcatagtggttcctcgagtcctgggaggcgtcgcatgccgtgctgggtctcatcgccgtcatctccatccagcgcgccattcacaagatccttatcgccgttttcctcagtcgcgagttcaagcacgacgagacgaacagggcctggtggactggtcgctggtatggccgtggcctcggcacgcacgccatgtcgcagccggcgcgtgagttcgtcgtcaagatcatcgagttgtcgctttggagctcggatctcatactcggccacatcctgctgttcatgcttactccggccgtcctcatcccgtacttcgaccgtttgcacgccatgatgctctgtacgtcgtgtctcattgtctgtgttggtcatactcttaccctctcttagtctggctgcgtccctcgaagcaaatccgcgcgcctctgtactcgatcaagcagaagaggcaaagacgctggattgtcagtgttcagtgccttattctatcagctcttactaacgtcttcatagatcatgaagtacggtactgtatacgttaccgtcatcgcgatcttcgtcgcgctcatcgcgcttcgtgagtttccttgctatttttcgtacctgagcgtcgctgacccctttcccagccctcgtattccgacacactctaaaggtcgagtgctccctttgcgacagcttgtaatatcggactcgtatatatctagacttctccgcaccatgtgtagctgacgcttgggtatacttcgcggtgccgagctaattgtcgacggacattctccatcgttgagtgcagcgacgtcgggtggtttacgacacggacacttttcattgtaccctctacgaatgcaagaactctcttacgaccagtacctatgtgctaagccgtcgcctgttcaggatcatacatacatacgtttctagataccttacagttaggcctattcagggagagtctgcataaaa

SEQ ID NO: 12 - protein, 1783 aa, S. commune
MPRPGGTSAEGGYASSPSMETTPSDPFGTANGAPRRYYDNDSEEYGPGRRDTYASDSSNQGLTDPGYYDQNGAYDPYPTGDTDSDGDVYGQRYGPSAESLGTHKFGHSDSSTPTFVDYSASSGGRDSYPAWTAERNIPLSKEEIEDIFLDLTQKFGFQRDSMRNMFDFTMQLLDSRASRMTPNQALLTLHADYIGGQHANYRKWYFAAQLDLDDAVGQTQNPGLNRLKSTRGSGKRPRHEKSLNTALERWRQAMNNMSQYDRLRQIALYLLCWGEAAQVRFMPECLCFIFKCADDYYRSPECQNRMEPVPEGLYLRTVVKPLYRFVRDQGYEVVEGKFVRRERDHDQIIGYDDVNQLFWYPEGIARIVLSDKSRLVDLPPAQRFMKFDRIEWNRVFFKTFYETRSFTHLLVDFNRIWVVHIALYFFYTAYNSPTIYAINGNTPTSLAWSATALGGAVATGIMILATIAEFSHIPTTWNNTSHLTRRLAFLLVTLGLTCGPTFYVAIAESNGSGGSLALILGIVQFFISVVATALFTIMPSGRMFGDRVAGKSRKYLASQTFTASYPSLPKHQRFASLLMWFLIFGCKLTESYFFLTLSFRDPIRVMVGMKIQNCEDKIFGSGLCRNHAAFTLTIMYIMDLVLFFLDTFLWYVIWNSVFSIARSFVLGLSIWTPWRDIFQRLPKRIYAKLLATGDMEVKYKPKVLVSQIWNAIIISMYREHLLSIEHVQKLLYHQVDTGEAGKRSLRAPPFFVAQGSSGGSGEFFPPGSEAERRISFFAQSLSTEIPQPIPVDAMPTFTVLTPHYSEKILLSLREIIREEDQNTRVTLLEYLKQLHPVEWENFVKDTKILAEESAMFNGPSPFGNDEKGQSKMDDLPFYCIGFKSAAPEYTLRTRIWASLRAQTLYRTVSGMMNYAKAIKLLYRVENPEVVQQFGGNTDKLERELERMARRKFKFLVSMQRYSKFNKEEHENAEFLLRAYPDLQIAYLEEEPPRKEGGDPRIFSALVDGHSDIIPETGKRRPKFRIELPGNPILGDGKSDNQNHAIVFYRGEYLQLIDANQDNYLEECLKIRNVLAEFEEYDVSSQSPYAQWSVKEFKRSPVAIVGAREYIFSEHIGILGDLAAGKEQTFGTLTARNNAFLGGKLHYGHPDFLNALYMNTRGGVSKAQKGLHLNEDIYAGMNAVGRGGRIKHSEYYQCGKGRDLGFGTILNFQTKIGTGMGEQILSREYYYLGTQLPIDRFLTFYYAHPGFQINNMLVILSVQVFIVTMVFLGTLKSSVTICKYTSSGQYIGGQSGCYNLVPVFQWIERCIISIFLVFMIAFMPLFLQELVERGTWSAIWRLLKQFMSLSPVFEVFSTQIQTHSVLSNLTFGGARYIATGRGFATSRISFSILFSRFAGPSIYLGMRTLIMLLYVTLTIWTPWVIYFWVSILSLCIAPFLFNPHQFVFSDFLIDYREYLRWMSRGNSRSHNNSWIGYCRLSRTMITGYKKKKLGHPSEKLSGDVPRAGWRAVLFSEIIFPACMAILFIIAYMFVKSFPLDGKQPPSGLVRIAVVSIGPIVWNAAILLTLFLVSLFLGPMLDPVFPLFGSVMAFIAHFLGTIGMIGFFEFLWFLESWEASHAVLGLIAVISIQRAIHKILIAVFLSREFKHDETNRAWWTGRWYGRGLGTHAMSQPAREFVVKIIELSLWSSDLILGHILLFMLTPAVLIPYFDRLHAMMLFWLRPSKQIRAPLYSIKQKRQRRWIIMKYGTVYVTVIAIFVALIALPLVFRHTLKVECSLCDSL

SEQ ID NO: 13 - DNA, 5223 bp, S. commune
atgtccggtccaggatatggcaggaatccattcgacaatcccccgcccaacagaggtccctatggccagcagccaggtttcccggggcccggccctcggccttacgactcggacgcggacatgagccagacctatggcagcacaaccaggctcgccggcagtgccggttacagcgacagaaacggcagcttcgacggcgaccgctcctacgcgccctcaattgactcgcgcgccagcgtgcccagcatatcgcccttcgcagacccgggtatcggctctaatgagccgtatcccgcttggtcggtcgaacgccagatccccatgtccacggaggagattgaggatatcttcctcgacctcacccaaaagtttggcttccagcgcgactccatgcggaatacgttcgacttcatgatgcacctccttgattcccgtgcctcgcgcatgacgcccaaccaagctctgctcacgcttcacgccgactacattggtggccagcacgccaactataggaagtggtatttcgccgctcagctcaacctcgatgacgcggtcgggcaaaccaataaccccggtatccagcgcttgaagaccatcaagggcgctacgaagaccaagtcgctcgacagcgcactcaaccgctggcgcaatgcgatgaacaacatgagccagtacgatcgcctccggcaaattgcgctctatctcctctgctggggagaagcaggcaacatccgtctggcgcccgagtgcttgtgcttcatcttcaagtgcgcggacgactactacagaagtcccgagtgtcagaaccggatggaccccgtgccggaagggctgtacctccagacggtcatcaagccgctctatcgcttcctacgtgatcaggcgtacgaagtcgttgatgggaagcaagtgaagcgcgagaaggaccacgaccagattatcggttatgacgacgtcaaccagttattctggtatccggaaggtttggctaagatcgtcatgtcggacaacacacgacttgtagatgtacctccggcgcagcggttcatgaagttcgccaagatcgagtggaaccgcgtcttcttcaagacgtactttgagaagcgctctactgcccatctcctggtcaacttcaaccgtatatggatcctccacgtctcgatgtacttcttctacacggcattcaactctccacgagtctacgcgccgcacggcaaactcgacccctcccctgagatgacctggtccgcgactgcccttggaggcgctgtgtccaccatgatcatgatccttgccactatcgcggagtacacctacatccccacgacatggaacaatgcgtcgcacctcaccacgcggctcattttcctcctggtcatcctcgcgctcactgctggaccaacattctatatcgccatgatagacggacgcacggacatcggccaagtaccactcatcgtggccatagtgcagttcttcatctccgtcgtcgccaccctcgctttcgctaccatcccttctggtcgcatgttcggcgaccgtgtggctggcaagtcaagaaagcacatggcatcgcagacgttcacagcgtcgtacccgtccatgaagcggtcatctcgcgtagcgagtatcatgctgtggcttttggtctttggctgcaaatacgtcgagtcttacttcttcttgacgtcctccttctccagcccgatcgcggtcatggcgcgtacgaaggtacagggctgcaacgaccgtatcttcggcagccagctgtgcacgaatcaggtcccgttcgcgctggcaatcatgtacgtgatggacctggtactgttcttcctggacacgtacctgtggtacatcatctggctggtgatcttctcgatggtgcgcgcgttcaagcttggtatctcgatctggacgccctggagcgagatcttcacccgcatgccgaagcgtatctacgcgaagctgctggcgacggccgagatggaggtcaagtataagcccaaggtgctcgtctcgcaaatctggaacgcggtcatcatctccatgtaccgggagcatctcttgtccatcgagcacgtccagcgcctgctataccaccaggttgatggtccagacggtcgccgcaccctcagggcaccgccgttcttcaccagccagcgaactgcgaagccaggcctgttcttccctcctggtggcgaggctgagcgccgtatctcgttctttgcctcatcgctgacgaccgcgctccctgagcctctgccgatcgacgccatgcccaccttcaccgtgctcgttccccattactcggagaagattctgctcagtctgcgcgagattattcgcgaggaggaccagaacacccgcgtcaccttgctggagtacctcaagcagctccaccctgtcgaatgggacaacttcgtcaaggacaccaagatcttggcggaagagtcgggcgacgtccaggacgagaagcgcgcgcgcacggacgacttgccgttctactgcatcgggttcaagacctcgtcaccagagtacaccctgcgtacgcgtatctgggcttcactgcgcgcacagacgctgtaccgcacggtctccggtatgatgaactactccaaggcgatcaagctcctctatcgcgtcgagaacccggatgtcgttcatgccttcggtgggaacacggaacgtcttgaacgcgagcttgagcgcatgtctcgccgcaagttcaagttcgtcatctcgatgcagcggtactctaagttcaacaaggaggagcaagagaacgccgaattccttctgcgcgcgtacccggatttgcagatcgcgtacctcgatgaagagcccggtcccagcaagagcgacgaggttcggttgttttcgacactcatcgatggacactccgaggtggatgagaagaccggccgccgcaagcccaagttccgcattgagctgcccggtaaccccatcctcggtgacgggaagtcggataaccagaaccacgccattgtcttctaccgcggcgagtacatccaggtcatcgacgctaaccaggacaattacctggaagagtgtctcaagatccgtaacgtcctgggcgagtttgaggaatactccgtgtcgagccagagcccgtacgcacagtggggccacaaggagttcaacaagtgccccgtcgctatcctgggttctcgcgagtacatcttctcggagaacatcggtatcctcggtgacatcgccgccggcaaggaacagacgttcggtaccattacggcgcgtgcgcttgcgtggatcggcggcaagctgcattacggtcacccggatttcctcaatgcgacgttcatgacgacgcgtggtggcgtgtcaaaagcgcagaagggcttgcatctcaacgaggatatcttcgctggtatgaccgccgtgtcccgcggagggcgcatcaagcacatggagtactaccagtgcggcaaaggtcgtgatctcggtttcggcacgatcttgaacttccagacgaagatcggtactggtatgggcgagcagctcctctcgcgcgagtactactacctgggcacgcaattgcctatcgaccggttcttgacgttctactacgcgcacgctggtttccacgtcaacaacatcctggtcatctactccatccaggtcttcatggtcaccttgctgtacctgggcacattgaacaagcagctgttcatctgcaaggtcaactccaatggccaggttcttagtggacaagctgggtgctacaacctcatcccggtcttcgagtggattcgccggagtatcatctccatcttcttggtgttcttcatcgccttcttgcctctattcttgcaagagctgtgcgagcgcggaacgggaaaggcgttgctgcgtctcgggaagcacttcttgtcactgtcgcccattttcgaagtgttctccacccagatttactcgcaggcgctcttgaacaacatgagcttcggtggtgcgcgctacatcgccacaggtcgtggtttcgcgactagtcgcatacccttcaacatcctctactcgcgtttcgcgccgccaagcatctacatgggcatgcgtaacctgctgctcctgctgtacgcgacgatggccatttggatcccgcacctgatctacttctggttctccgtcctctccctctgcatcgcgccattcatgttcaatccgcatcaattctcgtacgccgacttcatcatcgactaccgggagttcttgcgctggatgtcgcgcggtaactcgcgaacgaaggcgagcagctggtacggatactgccgtctgtcgcgtaccgcgattactgggtacaagaagaagaagctgggacacccgtcggagaagctgtcgggcgacgtaccgcgtgcgccgtggaggaacgttatcttctcggagatcctgtggcccatcggcgcgtgcatcatcttcatcgtcgcgtacatgttcgtcaagtcgttccccgacgagcagggcaacgcgccgccgagcccgctggtccggattctgctcatcgcggttggccctactgtgtggaacgcggcggtgctcataacgctgttcttcctgtcgctcttcctgggcccgatgatggatggctgggtcaagttcggctcggtcatggcggcccttgcgcatggcctggcgcttataggcatgctcacgttctttgagttcttctggttccttgagctctgggatgcctcgcacgccgtgctcggcgtcatcgctatcattgccgttcagcgcgggatccagaagatcctcattgccgtcttcctgacgcgtgagtacaagcacgacgagacgaaccgcgcgtggtggacaggtaaatggtatggacgcgggctgggtacctcggccatgtcccagccggcgcgcgagttcatcgtgaagatcgtggagatgtcgttgtggacgtcggacttcctgcttgcgcacctgttgctcatcatcttgacggtgccgctactgctgccgttcttcaactcaattcattcgacgatgcttttctggttgcgcccttcgaagcagattaggcaacctctgttctccaccaagcagaagcggcaacggcgatggattgtcatgaagtataccgtggtatatctcgtggtggtggctttcctcgtcgcgctcatcgctctgcccgccctcttccgcgagagcatccacttcaactgcgagatctgccagagtatatag

SEQ ID NO: 14 - protein, 1740 aa, S. commune; identical to SEQ ID NO: 2

SEQ ID NO: 15 - DNA, 5352 bp, S. commune
atgccgaggccgggcggcaccagcgcagaaggcggctacgcatcatcgccgtcgatggagacgacccccagcgatcccttcggaaccgcgaacggcgcgccccgccgctactacgacaatgattctgaggagtacggacctggccgtagagacacctacgcgtccgacagcagtaatcagggcctcacggacccgggctactacgaccagaatggcgcctatgatccctatccgaccggggacaccgattccgacggcgacgtctacggccagcgatatggaccctcagcagagtcgcttggcacccacaagttcggccattccgattcatccacgccgacttttgtcgactacagcgcatcctccggcgggagggattcgtaccctgcatggactgccgaacgcaacatcccgctgtccaaggaggagatcgaggacatcttcctcgatttgacgcagaagtttggctttcagcgggattccatgcggaatatgttcgacttcaccatgcagctgcttgacagccgagcgtctcgtatgacccccaaccaggcgctcctcaccctccacgccgactacattggtggccagcatgcgaactaccggaagtggtacttcgcggcgcagctcgaccttgacgacgccgtgggacaaactcagaatccgggtctcaaccgcctcaagtccactcgcggatcgggcaagcgaccacgccatgaaaagtcgctgaacacggcattggagcgctggcggcaagccatgaacaacatgtcgcagtatgaccgcttacgccagatcgcgctctacctgctctgctggggcgaagcggcgcaagtgcgattcatgcccgagtgcttgtgcttcatcttcaagtgcgccgacgactactatcgttcgccggagtgccagaacaggatggagccggtaccggagggtctctacctgaggacggtcgtaaagccgctctacagatttgtccgggatcaaggctatgaggtggtggagggaaaattcgtacggcgggaacgggatcacgaccaaatcattggttacgatgacgtgaatcagctgttctggtacccggagggaattgcccgtatcgtcctgtcggacaagagtcgtctagtcgacctccccccagcacagcgcttcatgaagttcgaccgtatcgagtggaatcgcgtcttcttcaagacgttttacgagactcgatccttcacgcatcttttggtcgacttcaaccgtatctgggtcgtgcacatcgctctctacttcttctacactgcatacaactcccccacgatctacgccatcaacggcaacacaccgacgtctctggcttggagcgcgactgcgctcggcggtgcggtagcgacaggtatcatgatcctcgccacgatcgccgagttctcgcacatccccacgacatggaacaacacctcgcatctgactcgccgcctcgccttcctcctcgtcacgctcggcctcacatgtggtccgacgttctacgtcgcgattgcagagagcaacgggagcggcggctctttggccttgattctcggtatcgtccagttcttcatctccgtcgtggcaactgcgctcttcactatcatgccttctggtcgtatgttcggcgaccgtgtcgcaggcaagagtcgcaagtatctcgccagccagacgttcacggccagctacccgtcgttgcccaagcaccagcggttcgcctcactcctgatgtggttcctcatcttcgggtgcaagttgacggagagttacttctttctgacgctgtccttccgcgaccctatccgcgtcatggtcggcatgaagatccagaactgcgaggacaagattttcggcagcggcctttgcaggaatcacgcagcattcaccctcacgatcatgtacatcatggacctcgtcttgttcttcctcgacaccttcctttggtatgtcatctggaactcggttttcagtatcgcacgctctttcgtactcggcctttcgatctggacaccgtggagagacatcttccagcgtctgccgaagcggatctacgcgaagcttctggcgactggcgacatggaggtcaagtacaagcccaaggtcttggtctcgcaaatctggaacgccatcatcatctccatgtaccgcgagcacttgctctctattgagcacgtccagaagctcctgtaccaccaagtggacactggcgaagccggcaagcggagtcttcgcgcgcctccgttcttcgtcgcgcagggcagcagcggtggctcgggcgagttcttcccgcctggcagcgaggccgagcgtcgtatctctttcttcgcgcagtcgctttctacggagattcctcagcccatcccggtcgacgccatgccgacgttcacggtgcttacgcctcactacagcgagaagatccttctctctctccgtgaaattatccgcgaggaggaccagaacactcgcgttacgttgctcgagtacctgaagcagctgcatccggtcgagtgggagaatttcgtcaaggacactaaaattttggccgaggagtccgctatgtttaacggtccgagtcctttcggcaacgacgagaagggtcagtccaagatggacgatctaccgttctactgcatcggtttcaagagcgccgcgcccgagtacaccctccgcacccgtatctgggcgtccctgcgcgcgcagacgctgtaccgcacggtctccggcatgatgaactatgcgaaggcgatcaagctgctctaccgcgttgagaacccggaggtcgtacaacagttcggcggcaacacggacaagctcgagcgcgagttggagcggatggcgcgacggaagttcaagttcctcgtgtccatgcagcgctactcgaagttcaacaaggaggagcacgagaacgccgagttcttgctccgcgcgtacccggacttgcagatcgcgtacctcgaggaagagccccctcgcaaggagggcggcgatccacgcatcttctctgccctcgtcgacggccacagcgacatcatcccggagaccggcaagcggcgccccaagttccgtatcgagctgcccggtaaccccattctcggtgacggtaaatccgacaatcagaaccacgctatcgtcttctaccgcggcgagtacctccagcttatcgacgccaaccaggacaactacctcgaggagtgcttgaagatccgtaacgtgctcgccgagtttgaggagtacgacgtctccagccagagcccgtacgcgcagtggagtgtcaaggagttcaagcgctctccggtcgccatcgtcggtgcacgcgagtacatcttctcagagcacatcggtatcctcggtgatctggcggctggcaaggaacagacgttcggtacgctcacggcacgcaacaacgccttccttggcggcaagctgcactacggtcaccccgatttcctcaacgccctctacatgaacacgcgcggtggtgtctccaaggcgcagaagggtctccatctcaacgaggatatctacgccggtatgaacgcggtcggtcgcggtggacgcattaagcacagcgagtactatcagtgcggcaagggtcgtgacctcggtttcggcaccatcttgaacttccagaccaagatcggtacgggtatgggcgagcagatcctctcgcgcgagtactactatctcggaacacaactgcccatcgatcgcttcctcacgttctactacgcgcacccgggtttccagatcaacaacatgctggtcatcctctccgtgcaggtcttcatcgttaccatggtcttcctcggtaccttgaagtcttcggtcacgatctgcaagtacacgtccagcggtcagtacatcggtggtcaatccggttgctacaacctcgtcccggtcttccagtggatcgagcgctgcatcatcagcatcttcttggtgttcatgatcgctttcatgccgctcttcctgcaagaactcgtcgagcgcggtacctggagtgccatctggcgtctgctcaagcagtttatgtcgctgtcgcctgtcttcgaggtgttctccacccagattcagacgcactccgtgttgagcaacttgacgttcggtggtgcgcgttacatcgctaccggtcgtgggttcgccaccagtcgtatcagcttcagcatcttgttctcgcgtttcgcaggcccgagtatctacctcggcatgcgcacgctcattatgctgctctacgtgacgttgacgatctggacgccatgggtcatttacttctgggtttccattctctcgctctgcatcgcgccgttcttgttcaacccgcatcaattcgtattctcggacttcctcatcgactacagggaatacctgcggtggatgtcgcgtggcaactcgcgctcgcacaacaactcctggattgggtactgccggttgtcccgcacgatgatcactgggtacaagaagaagaagctgggccacccgtcggagaagctttccggcgacgttcctcgtgcaggctggcgcgccgtcttgttctcggagatcatcttcccggcgtgcatggccatcctcttcatcatcgcgtacatgttcgtcaagtcgttccctctcgacggcaagcagcctccctccggcctcgttcgcatcgccgtcgtgtctatcggccccatcgtgtggaacgccgccatcctgttgacgctcttccttgtgtcgttgttcctcggccccatgctcgacccggtcttccccctcttcggttccgttatggccttcatcgcgcatttccttggcacaatcggaatgattgggttcttcgagttcctgtggttcctcgagtcctgggaggcgtcgcatgccgtgctgggtctcatcgccgtcatctccatccagcgcgccattcacaagatccttatcgccgttttcctcagtcgcgagttcaagcacgacgagacgaacagggcctggtggactggtcgctggtatggccgtggcctcggcacgcacgccatgtcgcagccggcgcgtgagttcgtcgtcaagatcatcgagttgtcgctttggagctcggatctcatactcggccacatcctgctgttcatgcttactccggccgtcctcatcccgtacttcgaccgtttgcacgccatgatgctcttctggctgcgtccctcgaagcaaatccgcgcgcctctgtactcgatcaagcagaagaggcaaagacgctggattatcatgaagtacggtactgtatacgttaccgtcatcgcgatcttcgtcgcgctcatcgcgcttcccctcgtattccgacacactctaaaggtcgagtgctccctttgcgacagcttgtaa

SEQ ID NO: 16 - protein, 1783 aa, S. commune; identical to SEQ ID NO: 12

SEQ ID NO: 17 - DNA, 620 bp, S. commune
atcgccattgtaagccgcagacgggcacgcttccaacccccatcgatgggcgctcgatgtccatctcatcggcgactcatcattgtatctcgcgcagtcccatccctcgccgctcgcctgtagtttatgctatttatctttgcaccagtcgttgtattactccctcgtcgtgtagaaagtaccagataaaatgcatgtaatcctaatgaaatttgcacgacacgaagatccggcagggttgtgggcaaggggcagcgggaacgaatggatggcggggtacagcgagtacccggcagtgccacagtcagtgtcacacacgtgactgattgtccattagcgtgaccgataacatcgatcaaaaattttatttcagaggacgataaataagggccgacggtgcgcgtccgtctttctctcaaccctcatcttcctctcgtctctcactcttcccccctccaccactaccaagtaagttcaaacttcctctcatcgcctttgcacacatcgcctacgccccatctctctccatctgcctcgcgaacggcgcccccatcgtcgctttcccgcgcgagatcttgtgcgatctagtttactgacaatctcacctagaaaacatcaaa

SEQ ID NO: 18 - DNA, 440 bp, S. commune
atccaagtccggtggcaaggtcaccaagtccgccgagaaggccgccaagaagaagtaaatgtagatgtacatatgtattttctcattccgtttccttcctcttgttgttgtttcactggtcctctcgtgctcgctcgcatcgcatacagccattgttgtcaccactataacttcacgcattctgtatttcatgccaggcgacggggtgttcctgccaggcctgtcgcttgttgtaacgctaatgaaaagtcacgagtagtggacgaacgacgatgtatttctatgtgctgtagcgattatccatttcgagttcgccatcgagctctcttcaaacctaggtgcgacgttgtgaatgcagtagcaagtgcagagtattgcagactcgtccattgatgataacttcaagctacgtcagagccagatgctactgaacccgggcc

SEQ ID NO: 19 - DNA, 36 bp, Artificial Sequence
  Ura_forw (NotI) primer
ataagaatgcggccgctccagctcgaccttgcgccg

SEQ ID NO: 20 - DNA, 30 bp, Artificial Sequence
  Ura_rev (XbaI) primer
ctagtctagaggatccgacgtggaggagcc

SEQ ID NO: 21 - DNA, 30 bp, Artificial Sequence
  TefP_forw (XbaI) primer
ctagtctagaatcgccattgtaagccgcag

SEQ ID NO: 22 - DNA, 30 bp, Artificial Sequence
  TefP_rev (SpeI) primer
ctagactagttttgatgttttctaggtgag

SEQ ID NO: 23 - DNA, 30 bp, Artificial Sequence
  TefT_forw (SalI) primer
acgcgtcgaccaagtccggtggcaaggtca

SEQ ID NO: 24 - DNA, 31 bp, Artificial Sequence
  TefT_rev (SalI) primer
ccgacgtcgacgggttcagtagcatctggct

SEQ ID NO: 25 - DNA, 32 bp, Artificial Sequence
  TefT_forw (EcoRV) primer
catggtgatatccaagtccggtggcaaggtca

SEQ ID NO: 26 - DNA, 32 bp, Artificial Sequence
  TefT_rev (ApaI) primer
ccgtatgggcccgggttcagtagcatctggct

SEQ ID NO: 27 - DNA, 30 bp, Artificial Sequence
  GS1_forw (SpeI) primer
ctagactagtcccgtccctcaaggccgttc

SEQ ID NO: 28 - DNA, 36 bp, Artificial Sequence
  GS1_rev (SalI) primer
aatggccgacgtcgacatggtatatgcaatgctatg

SEQ ID NO: 29 - DNA, 30 bp, Artificial Sequence; identical to SEQ ID NO: 21
  Fusion TefP_GS1_forw (XbaI) primer

SEQ ID NO: 30 - DNA, 36 bp, Artificial Sequence; identical to SEQ ID NO: 28
  Fusion TefP_GS1_rev (SalI) primer

SEQ ID NO: 31 - DNA, 30 bp, Artificial Sequence
  GS2_forw (SpeI) primer
ctagactagtctgtccaaagaagagatcga

SEQ ID NO: 32 - DNA, 33 bp, Artificial Sequence
  GS2_rev (EcoRV) primer
tacatgcgatatcttttatgcagactctccctg

SEQ ID NO: 33 - DNA, 1614 bp, S. commune
tccagctcgaccttgcgccgcttggagtaacgttcagcgtcttcgtcgtcctcgtcgcgctcgtgtacgatgatgggctcagccatggcaggtatacaagctcagagtcaatgggggacgaggtctcaagccgtgaaagtcgtcgtcgaacaacgtcaagttcgagacggaccagagttggatttcgtgattagatctacgctcgatcacagaatgatcaaagaacaaagcttgccaaaaggggatctcccatcaacttcaacttgccccaaaccatcatgaccgccgctcataagctcacatacggtcagcgcgctgcaaggttcaccaatcccgcggcgaaagccctgctggaaaccatggagcgcaagaagagcaatctatccgtcagcgtcgacgtcgtaaaatccgccgatctgctcgctattgtcgataccgtcgggccctatatctgtctgataaaggcattgcactgtcgcttgcggtcttgggatgctgcttatactctatgaagacccatgtggatgttgtcgaagacttcgactcgtcgctcgtcaccaagcttcaggctctggccgagaagcatgatttcctcatctttgaggacagaaaattcgccgacataggtctgtccgtcgaatctctatcgatgtcaactctgatgacttgcacaggcaacaccgtcgctctgcagtactctagtggcgtgcacaaaattgccagctggtcgcacatcacgaacgcacaccctgttccaggaccgtcaatcatcagtggcctcgcatcggtaggacaacccctcggtcgcggactcctcctgctcgcagagatgagcacgaagggctcacttgcgacaggcgcgtacactgaagccgccgtccagatggcaagggagaaccgcggcttcgtcatcgggttcatcgcccaacggcggatggatggtattggcgcgcctccaggggtgaatgtcgaggacgaggattttcttgtcttgacaccaggtgtcggactcgatgtgaagggcgatgggatggggcagcaatacaggacgccgaagcaagtggtacaggaagatgggtgcgatgtaatcatcgtgggtcgcgggatttatggcaaggacccatcgaaggtggaagagatacggaggcaggcagagcgttaccaggctgcaggatgggcggcgtacattgagagggtcaacgccttggtatagctaatctgatcggtgttgtcttgttaagcgtcaggctcaatggaacgctttggacgagcggagagtaacttgaattagcagtgtatacttcgggcaaatcaatcgtgataaatacaagagcacgctcacgcacgtccaatctccctcaaaatctccatctttctcgcctcattcaccttcctgaacccagccggcgacatctcgaacagaccatgcccacccgacagcgcacgcagcctattcgagtagtccagcatccggctgagcggcgccaccgcctgcaccgcgcgcttcatcttcacgcccgccgcctccctcgccgcagtgccgccagagggcgacacccactccgggggcacgtacacgccgtccgcagggtacggctcctccacgtcggatcc

SEQ ID NO: 34 - protein, 278 aa, S. commune
MTAAHKLTYGQRAARFTNPAAKALLETMERKKSNLSVSVDVVKSADLLAIVDTVGPYICLIKTHVDVVEDFDSSLVTKLQALAEKHDFLIFEDRKFADIGNTVALQYSSGVHKIASWSHITNAHPVPGPSIISGLASVGQPLGRGLLLLAEMSTKGSLATGAYTEAAVQMARENRGFVIGFIAQRRMDGIGAPPGVNVEDEDFLVLTPGVGLDVKGDGMGQQYRTPKQVVQEDGCDVIIVGRGIYGKDPSKVEEIRRQAERYQAAGWAAYIERVNALV

(19) United States
(12) Patent Application Publication
(10) Pub. No.: US 2015/0152453 A1
(43) Pub. Date: Jun. 4, 2015

(54) GENETICALLY MODIFIED MICROORGANISMS CAPABLE OF PRODUCING BETA-GLUCANS AND METHODS FOR PRODUCING BETA-GLUCANS

(71) Applicants: BASF SE, Ludwigshafen (DE); Wintershall Holding GmbH, Kassel (DE)

(72) Inventors: Beata Brockmann, Morristown, NJ (US); Andrea Herold, Weinheim (DE); Oskar Zelder, Speyer (DE); Stefan Haefner, Speyer (DE); Christian Fleck, Sandhausen (DE); Hartwig Schröder, Bussloch (DE); Mari Granström, Kerava (FI); Julia Kristine Schmidt, Heidelberg (DE)

(21) Appl. No.: 14/412,212

(22) PCT Filed: Jul. 3, 2013
(86) PCT No.: PCT/EP2013/064024

§ 371 Date: Dec. 31, 2014

(30) Foreign Application Priority Data
Jul. 4, 2012 (EP) 12174882.6

(51) Int. Cl. C12P 19/04; C12N 9/10

(52) U.S. Cl. CPC C12P 19/04; C12N 9/1051; C12Y 204/01034

(57) ABSTRACT

The present invention relates to genetically modified microorganisms capable of producing beta-glucans, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain. The present invention also relates to the use of a polynucleotide encoding a polypeptide having 1,3-β-D-glucan synthase-activity or the use of such a polypeptide for producing β-glucans. Furthermore, the present invention relates to methods for producing β-glucans comprising the introduction of a promoter upstream of a polynucleotide encoding a polypeptide having 1,3-β-D-glucan synthase-activity thereby increasing the expression of said polynucleotide, or a polynucleotide encoding a polypeptide having 1,3-β-D-glucan synthase-activity into a microorganism being able to synthesize β-glucans.

The present invention relates to genetically modified microorganisms capable of producing beta-glucans (herein also referred to as β-glucans), characterized said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain. D-glucan synthase-activity compared to a corresponding non-modified control microorganism of the same strain. The present invention also relates to the use of a polynucleotide encoding a polypeptide having 1,3-β-D-glucan synthase-activity or the use of such a polypeptide for producing β-glucans. Furthermore, the present invention relates to methods for producing β-glucans comprising the introduction of a polynucleotide encoding a polypeptide having 1,3-β-D-glucan synthase-activity into a microorganism being able to synthesize β-glucans. In context of the present invention, the term “β-glucans” may particularly comprise polymers consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

β-glucans are known well-conserved components of cell walls in several microorganisms, particularly in fungi and yeast (Novak, Endocrine, Metabol & Immune Disorders—Drug Targets (2009), 9: 67-75). Biochemically, β-glucans comprise non-cellulosic polymers of β-glucose linked via glycosidic β(1-3) bonds exhibiting a certain branching pattern with β(1-6) bound glucose molecules (Novak, loc cit). A large number of closely related β-glucans exhibit a similar branching pattern such as schizophyllan, scleroglucan, pendulan, cinerian, laminarin, lentinan and pleuran, all of which exhibit a linear main chain of β-D-(1-3)-glucopyranosyl units with a single β-D-glucopyranosyl unit (1-6) linked to a β-D-glucopyranosyl unit of the linear main chain with an average branching degree of about 0.3 (Novak, loc cit; EP-B1 463540; Stahmann, Appl Environ Microbiol (1992), 58: 3347-3354; Kim, Biotechnol Letters (2006), 28: 439-446; Nikitina, Food Technol Biotechnol (2007), 45: 230-237). Although these β-glucans are structurally closely related, their respective microbial producers are not. Examples of microorganisms producing these structurally closely related β-glucans are Schizophyllum commune (for schizophyllan; Martin, Biomacromolecules (2000), 1: 49-60; Rau, Methods in Biotechnol (1999), 10: 43-55, DOI: 10.1007/978-1-59259-261-64); Sclerotium rolfsii, Sclerotium glucanicum, and Sclerotium delphinii (for scleroglucan; Survase, Food Technol Biotechnol (2007), 107-118); Porodisculus pendulus (for pendulan; EP-B1 463540); Botrytis cinerea (for cinerian; Stahmann, loc cit) Laminaria sp. (for laminarin; Kim, loc cit); and Lentinula edoles (for lentinan; Nikitina, loc cit). At least two of said β-glucans—schizophyllan and scleroglucan—even share an identical structure and differ only slightly in their molecular mass, i.e. in their chain length (Survase, loc cit).

Such β-glucans are widely used as thickeners and find application in several applications such as food industry and particularly oil industry (enhanced oil recovery, EOR) (Survase, loc cit). Also, such β-glucans are used in the pharmaceutical industry in tablet formulations and excipients as well as in immunotherapy as antiviral agents (Survase, loc cit).

Industrial production of β-glucans is mostly performed by fermentation processes using their natural microbial producers. Classical ways to improve β-glucan synthesis, e.g., of schizophyllan is based on manipulation of the development of S. commune (Rau, Habilitation, Braunschweig 1997). The most common approach is to convert dicaryotic cells via protoplast generation into monocarytic cells (Rau, Habilitation, Braunschweig 1997). Another approach is to cross different monocaryotic cells to form a new dicaryotic cell (Rau, Habilitation, Braunschweig 1997). Further possible approaches comprise, e.g., a classical random based mutagenesis using UV radiation, transposon mutagenesis or using suitable chemicals (e.g., nitrosoguanidin (NTG or N-methyl-N′-nitro-N-nitrosoguanidin), 2-aminofluorene (2-AF), 4-nitro-o-phenylenediamine (NPD), 2-methoxy-6-chloro-9-(3-(2-chloroethyl)aminopropylamino)acridine×2HCl (ICR-191), 4-nitroquinolone-N-oxide (NQNO), benzo[α]pyrene (B[alpha]p), or sodium azide (SA)) (Czyz, J Appl Genet (2002), 43(3): 377-389). Due to the rearrangement of genetic material within the crossing event it is possible to select strains exhibiting higher β-glucan (schizophyllan) productivity.

Yet, all of these approaches are undirected and do not allow targeted modification of the β-glucan producing microorganisms. In fact, results and efficiency of such approaches are not predictable and identification and selection of improved strains is labored and costly.

This technical problem has been solved by the means and methods described herein below and as defined in the claims.

In particular, as has been surprisingly found in context with the present invention, overexpression of 1,3-β-D-glucan synthase in a 3-glucan producing microorganism such as, e.g., S. commune or S. rolfsii leads to significant higher yields of the respective glucan. This finding was indeed unexpected given the fact that the biosynthetic pathway of β-glucan synthesis was only poorly understood and moreover, for most β-glucan producing microorganisms (such as Schizophyllum commune), there was no proposed β-glucan biosynthesis pathway available at all. Moreover, in context of those microorganisms whose β-glucan biosynthesis pathway was at least investigated (such as Pediococcus parvulus), enzymes such as α-phosphoglucomutase (α-PGM) and particularly UDP-glucose pyrophosphorylase (UGP) were assumed to represent a bottle-neck in β-glucan synthesis (Velasco, Int J Food Microbiol (2007), 115: 325-354). Accordingly, overexpression of these enzymes was assumed to increase the yields of β-glucan synthesis (Velasco, loc cit). Yet, as has been found in context with the present invention, overexpression of UGP in S. commune did not result in an increased yield of the β-glucan schizophyllan. In sharp contrast, as further described herein below and in the Examples, it has been found in context of the present invention that S. commune possesses two copies of 1,3-β-D-glucan synthase (genome sequence known from Ohm, Nature Biotech (2010), 28: 957-963) and, surprisingly, that overexpressing either of the two copies of 1,3-β-D-glucan synthase in S. commune leads to significant higher yields in the production of schizophyllan. Given that schizophyllan has a structure which is closely related to other β-glucans such as scleroglucan, pendulan, cinerian, laminarin, lentinan and pleuran (all of which are polymers consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3), it appears to be likely that overexpression of polypeptides having 1,3-β-D-glucan synthase activity in corresponding microorganisms as also described herein may therefore result in higher yields of those β-glucans.

Accordingly, the present invention relates to a genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain. Said polynucleotide may be endogenous or exogenous. For example, in context with the present invention, the overexpression of said polynucleotide may result from the introduction of a strong (e.g., constitutive or inducible) promoter upstream of said polynucleotide thereby increasing the expression level of said polynucleotide, or, preferably, from the introduction of at least one copy of a polynucleotide encoding a polypeptide having 1,3-β-D-glucan synthase-activity. In one embodiment, the present invention relates to a genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism contains at least one copy more of a polynucleotide encoding a polypeptide having 1,3-β-D-glucan synthase-activity compared to a corresponding non-modified control microorganism of the same strain. Said genetically modified microorganism is preferably capable of stably maintaining and expressing the additional polynucleotide encoding a polypeptide having 1,3-β-D-glucan synthase-activity. Said genetically modified microorganism may originate from a corresponding non-modified microorganism which preferably per se, i.e. naturally, contains a polynucleotide encoding a polypeptide having 1,3-β-D-glucan synthase-activity. Also, said genetically modified microorganism is preferably per se, i.e. before modification, able to produce a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3 as described herein. Into said genetically modified microorganism, a strong promoter or at least one polynucleotide encoding a polypeptide having 1,3-β-D-glucan synthase-activity may have been introduced. Non-limiting examples of means and methods for the introduction of a promoter sequence into a microorganism may comprise inter alia homologous recombination as known in the art (Ohm, World J Microbiol Biotechnol (2010), 26: 1919-1923). Also, in context with the present invention, the microorganism may have been modified such that more polypeptide having 1,3-β-D-glucan synthase-activity is expressed, e.g., by inserting a strong promoter as described herein, by adding introns into a polynucleotide encoding a polypeptide having 1,3-β-D-glucan synthase-activity, by adapting the codon usage, by improving the ribosomal binding site for better translational initiation, by introducing elements in the mRNA that stabilize it, or by inserting a polynucleotide with a higher transcription level having 1,3-β-D-glucan synthase-activity into the microorganism (cf. Ohm, loc cit).

In context with the present invention, the promoter may be introduced into said microorganism upstream of a polynucleotide encoding a polypeptide having 1,3-β-D-glucan synthase-activity and in a manner that said promoter increases or enhances the expression of said polynucleotide. Non-limiting examples of means and methods for the introduction of a polynucleotide into a microorganism may comprise transformation, transduction and transfection as commonly known in the art and as also exemplified herein (Sambrook and Russell (2001), Molecular Cloning: A Laboratory Manual, CSH Press, Cold Spring Harbor, N.Y., USA; Current Protocols in Molecular Biology, Update May 9, 2012, Print ISSN: 1934-3639, Online ISSN: 1934-3647; Methods in Yeast Genetics, A Laboratory Course Manual, Cold Spring Harbor Laboratory Press, 1990; van Peer, Applied Environ Microbiol (2009), 75: 1243-1247; Schmid, “Genetics of Scleroglucan Production by Sclerotium rolfsii”, dissertation Technische Universität Berlin, D83 (2008)). Non-limiting examples of means and methods for the introduction of a promoter sequence into a microorganism may comprise inter alia homologous recombination as known in the art (Ohm, World J Microbiol Biotechnol (2010), 26: 1919-1923). Strong promoters to be introduced upstream of a polynucleotide encoding a polypeptide having 1,3-β-D-glucan synthase-activity in context with the present invention may comprise, inter alia, constitutive promoters such as, e.g., tell promoter (translation and elongation factor 1a, S. commune, A. niger), gpdA promoter (glyceraldehyde-3-phosphate dehydrogenase, S. commune, A. niger; Schuren, Cur Genet (1998), 33: 151-156), trpC promoter (tryptophan biosynthesis, Aspergillus nidulans) or inducible promoters such as, e.g., glaA promoter (glucoamylase, A. niger), alcA (alcohol dehydrogenase, A. nidulans) cbhI (cellobiohydrolase I, Trichoderma reesei; Knabe, Dissertation “Untersuchung von Signalkomponenten der sexuellen Entwicklung bei dem Basidiomyceten Schizophyllum commune” (2008)) thiA (thiamine biosynthesis, Aspergillus oryzae) (Moore, Biotechnology, Vol. III, Genetic Engeneering of Fungal Cells, Enceclopedia of Life Support Systems (2007)). In context with the present invention, preferred promoters comprise tef1 and gdpA.

Generally, in context with the present invention, the polynucleotide encoding a polypeptide having 1,3-β-D-glucan synthase-activity may be introduced into the microorganism in any suitable form, e.g., comprised in a vector, a plasmid, or as naked nucleic acid as further described and exemplified herein. The polynucleotide introduced into the microorganism may then be exogenous, on a vector/plasmid within the microorganism (i.e. outside of the microbial chromosome(s)), or it may be incorporated into the microbial chromosome(s) by, e.g., random (ectopic) or homologous recombination or any other suitable method as known in the art. In context with the present invention, the polynucleotide encoding a polypeptide having 1,3-β-D-glucan synthase-activity which has been introduced into the microorganism (i.e. the additional copy to the natural endogenous polynucleotide encoding a polypeptide having 1,3-β-D-glucan synthase-activity of a corresponding unmodified strain) does not necessarily have to have the same nucleotide sequence as the natural endogenous polynucleotide encoding a polypeptide having 1,3-β-D-glucan synthase-activity of a corresponding unmodified strain, as long as it has 1,3-β-D-glucan synthase-activity as described herein.

In one embodiment of the present invention, the genetically modified microorganism is able to produce at least 1.5 times, more preferably at least 1.8 times more, more preferably at least 2.0 times more, and most preferably at least 2.2 times more β-glucan polymer compared to the corresponding non-modified control microorganism. In this context, production of, e.g., 1.5 times “more” β-glucan polymer may mean that a genetically modified microorganism produces an amount of β-glucan polymer which is 1.5 times higher compared to the amount of β-glucan polymer produced in the same time under the same conditions by a corresponding non-modified control microorganism. Alternatively, production of, e.g., 1.5 times “more” β-glucan polymer may mean that a genetically modified microorganism produces the same amount of β-glucan polymer as a corresponding non-modified control organism under the same conditions, however, 1.5 times faster. The amount of produced β-glucan polymer may be measured by methods known in the art and as also described herein.

Furthermore, the present invention relates to the use of a polynucleotide encoding a polypeptide having 1,3-β-D-glucan synthase-activity, or a polypeptide having 1,3-β-D-glucan synthase-activity, or of a genetically modified microorganism according to claim 1 for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

Furthermore, the present invention relates to a method of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, said method comprising the steps of:
(a) introducing (i) a strong (e.g., constitutive or inducible) promoter upstream of a polynucleotide encoding a polypeptide having 1,3-β-D-glucan synthase-activity thereby increasing the expression of said polynucleotide, or, preferably, (ii) a polynucleotide encoding a polypeptide having 1,3-β-D-glucan synthase-activity into a microorganism being able to synthesize said polymer;
(b) culturing said microorganism of (a) in a medium, thereby allowing said microorganism to produce said polymer; and
(c) optionally recovering said polymer from the medium.

As regards step (c) of the method described and provided herein, it is noted that in some cases (e.g., when β-glucans such as schizophyllan is used for oil drilling purposes), the culture broth may also be used directly (e.g., pumped into the drill hole), without previous recovery of the pure β-glucan. As such, the recovery step (c) is optional. Strong promoters to be introduced upstream of a polynucleotide encoding a polypeptide having 1,3-β-D-glucan synthase-activity in context with the present invention may comprise, inter alia, constitutive promoters such as, e.g., tef1 promoter (translation and elongation factor 1a, S. commune, A. niger), gpdA promoter (glyceraldehyde-3-phosphate, S. commune, A. niger), trpC promoter (tryptophan biosynthesis, Aspergillus nidulans) or inducible promoters such as, e.g., glaA promoter (glucoamylase, A. niger), alcA (alcohol dehydrogenase, A. nidulans) cbhI (cellobiohydrolase I, Trichoderma reesei) thiA (thiamine biosynthesis, Aspergillus oryzae), tef1 and gdpA being preferred promoters. In context with the present invention, the promoter is preferably introduced into said microorganism upstream of a polynucleotide encoding a polypeptide having 1,3-β-D-glucan synthase-activity and in a manner that said promoter increases or enhances the expression of said polynucleotide. Said promoter or said polynucleotide encoding a polypeptide having 1,3-β-D-glucan synthase-activity may be introduced in said microorganism by any means and methods known in the art, preferably in a manner that after introduction the promoter can increase the expression of said polynucleotide or that said polynucleotide can be stably maintained and expressed by the microorganism, respectively. Non-limiting examples of means and methods for the introduction of a promoter sequence into a microorganism may comprise, inter alia, recombinant homology as known in the art (Ohm, loc cit). Non-limiting examples of such methods for the introduction of a polynucleotide into a microorganism may comprise transformation, transduction and transfection as commonly known in the art and as also exemplified herein (Sambrook and Russell (2001), Molecular Cloning: A Laboratory Manual, CSH Press, Cold Spring Harbor, N.Y., USA; Current Protocols in Molecular Biology, Update May 9, 2012, Print ISSN: 1934-3639, Online ISSN: 1934-3647; Methods in Yeast Genetics, A Laboratory Course Manual, Cold Spring Harbor Laboratory Press, 1990; van Peer, Applied Environ Microbiol (2009), 75: 1243-1247; Schmid, “Genetics of Scleroglucan Production by Sclerotium rolfsii”, dissertation Technische Universität Berlin, D83 (2008)).

In context with the present invention, the strong promoter introduced into a microorganism upstream of a polynucleotide encoding a polypeptide having 1,3-β-D-glucan synthase-activity preferably increases the expression level of said polynucleotide at least 1.5-fold, more preferably at least 1.8-fold, more preferably at least 2.0-fold, and most preferably at least 2.2-fold. In this context, the expression level of a polynucleotide can be easily assessed by the skilled person by methods known in the art, e.g., by quantitative RT-PCR, Northern Blot (for assessing the amount of expressed mRNA levels), Dot Blot, Microarray or the like.

Generally, the term “overexpression” as used herein comprises both, overexpression of polynucleotides (e.g., on the transcriptional level) and overexpression of polypeptides (e.g., on the translation level). Accordingly, the present invention relates to a genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain. In context with the present invention, a genetically modified microorganism is to be considered as “overexpressing” a polynucleotide encoding a polypeptide having 1,3-β-D-glucan synthase-activity if it expresses at least 1.5-fold, more preferably at least 1.8-fold, more preferably at least 2.0-fold, and most preferably at least 2.2-fold of said polynucleotide compared to a non-modified control microorganism of the same strain. In this context, the expression level of a polynucleotide can be easily assessed by the skilled person by methods known in the art, e.g., by quantitative RT-PCR (qRT-PCR), Northern Blot (for assessing the amount of expressed mRNA levels), Dot Blot, Microarray or the like (see, e.g., Sambrook, loc cit; Current Protocols in Molecular Biology, Update May 9, 2012, Print ISSN: 1934-3639, Online ISSN: 1934-3647). Preferably, the amount of expressed polynucleotide is measured by qRT-PCR. Furthermore, in context with the present invention, a genetically modified microorganism is to be considered as “overexpressing” a polypeptide having 1,3-β-D-glucan synthase-activity if it expresses at least 1.5-fold, more preferably at least 1.8-fold, more preferably at least 2.0-fold, and most preferably at least 2.2-fold of said polypeptide compared to a non-modified control microorganism of the same strain. In this context, the expression level of a polypeptide can be easily assessed by the skilled person by methods known in the art, e.g., by Western Blot, ELISA, EIA, RIA, or the like (see, e.g., Sambrook, loc cit; Current Protocols in Molecular Biology, Update May 9, 2012, Print ISSN: 1934-3639, Online ISSN: 1934-3647). Preferably, the amount of expressed polypeptide is measured by Western Blot.

Generally, in context with the present invention, the polynucleotide encoding a polypeptide having 1,3-β-D-glucan synthase-activity may be introduced into the microorganism in any suitable form, e.g., comprised in a vector, a plasmid or as naked nucleic acid. The polynucleotide introduced into the microorganism may then be exogenous (e.g., on a vector or a plasmid) within the microorganism (i.e. outside of the microbial chromosome(s)), or it may be incorporated into the microbial chromosome(s) by, e.g., random (ectopic) or homologous recombination or any other suitable method as known in the art. In context with the present invention, the polynucleotide encoding a polypeptide having 1,3-β-D-glucan synthase-activity which has been introduced into the microorganism (i.e. the additional copy to the natural endogenous polynucleotide encoding a polypeptide having 1,3-β-D-glucan synthase-activity of a corresponding unmodified strain) does not necessarily have to have the same nucleotide sequence as the natural endogenous polynucleotide encoding a polypeptide having 1,3-β-D-glucan synthase-activity of a corresponding unmodified strain, as long as it has 1,3-β-D-glucan synthase-activity as described herein.

Methods for culturing microorganisms such as fermentation processes are known in the art and also described and exemplified herein (Kumari, Bioresource Technol (2008), 99: 1036-1043; Reyes, J Natural Studies (2009), 7(2), January-June). In context with the present invention, such methods allow the respective microorganism to grow and to produce the desired β-glucan as described and exemplified herein. Suitable media may comprise, e.g., coconut water as described in Reyes, loc cit. Furthermore, as known in the art, there are several media particularly suitable for particular microorganisms. For example, also in context with the present invention, suitable media for culturing S. commune comprise CYM medium (25 g agar (Difco), 20 g glucose (Sigma), 2 g trypticase peptone (Roth), 2 g yeast extract (Difco), 0.5 g MgSO₄×7 H₂O (Roth), 0.5 g KH₂PO₄ and 1 g K₂HPO₄ (both from Riedel-de Haën) per liter H₂O) (particularly useful for cultivation on solid support) or a medium comprising 30 g glucose (Sigma), 3 g yeast extract (Difco), 1 g KH₂PO₄ (Riedel-de Haën), 0.5 g MgSO₄×7H₂O (Roth) per liter H₂O (particularly useful for liquid cultures) as also described and exemplified herein. Further suitable media for culturing S. rolfsii are known in the art (Survase, Bioresource Technol (2006), 97: 989-993). The β-glucan produced in accordance to the present invention can be recovered by various methods known in the art and described herein (see also “Recommended Practices for Evaluation of Polymers Used in Enhanced Oil Recovery Operations, API Recommended Practice 63 (RP 63), 1^{st} Ed, American Petroleum Institute, Washington D.C., Jun. 1, 1990; Kumari, Bioresource Technol (2008), 99: 1036-1043).

In context with the present invention, the term “average branching degree about 0.3” may mean that in average about 3 of 10 β-D-(1-3)-glucopyranosyl units are (1-6) linked to a single β-D-glucopyranosyl unit. In this context, the term “about” may mean that the average branching degree may be within the range from 0.1 to 0.5, preferably from 0.2 to 0.4, more preferably from 0.25 to 0.35, more preferably from 0.25 to 0.33, more preferably from 0.27 to 0.33, and most preferably from 0.3 to 0.33. It may also be 0.3 or 0.33. Schizophyllan, scleroglucan, pendulan, cinerian, laminarin, lentinan and pleuran all have an average branching degree between 0.25 and 0.33; for example, scleroglucan and schizophyllan have an average branching degree of 0.3 to 0.33 (Survase, loc cit; Novak, loc cit). The average branching degree of a β-glucan can be determined by methods known in the art, e.g., by periodic oxidation analysis, methylated sugar analysis and NMR (Brigand, Industrial Gums, Academic Press, New York/USA (1993), 461-472).

In one embodiment of the present invention, the polymer to be produced is selected from the group consisting of schizophyllan, scleroglucan, pendulan, cinerian, laminarin, lentinan and pleuran. For example, the polymer may be schizophyllan or scleroglucan, particularly schizophyllan.

The microorganism of the present invention and as referred to and as employed in context with the present invention (hereinafter also referred to as “microorganism in context of the present invention”) may generally be a microorganism which is per se (i.e. naturally, in a non-modified state in context with the present invention) capable of synthesizing β-glucan polymers, particularly those polymers consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3. That is, such microorganisms preferably possess per se (i.e. naturally, in a non-modified state in context with the present invention) a polynucleotide encoding a polypeptide having 1,3-β-D-glucan synthase-activity. Non-limiting examples of microorganisms in context of the present invention are Schizophyllum commune, Sclerotium rolfsii, Sclerotium glucanicum, Sclerotium delphinii, Porodisculus pendulus, Botrytis cinerea, Laminaria sp., Lentinula edoles, and Monilinia fructigena. For example, the microorganism in context with the present invention may be S. commune or S. rolfsii, particularly S. commune.

The polynucleotide encoding a polypeptide having 1,3-β-D-glucan synthase-activity as referred to and to be employed in context with the present invention (hereinafter also referred to as the “polynucleotide in context of the present invention”) may be a 1,3-β-D-glucan synthase gene. For example, the polynucleotide in context of the present invention may comprise or may consist of a nucleic acid sequence which is at least 70%, preferably at least 75%, more preferably at least 80%, more preferably at least 85%, more preferably at least 90%, more preferably at least 95%, more preferably at least 96%, more preferably at least 97%, more preferably at least 98%, more preferably at least 99%, more preferably at least 99.5%, and most preferably 100% identical to SEQ ID NO: 1, 3, 5, 7, 9, 11, 13 or 15, provided that the polypeptide encoded by said polynucleotide has 1,3-β-D-glucan synthase-activity as further described and exemplified herein below. SEQ ID NO: 1 represents the nucleotide sequence of the gene of glucan synthase I of S. commune strain Lu15531 (obtained from Jena University (Germany) strain collection, Germany, Prof. E. Kothe; Jena University internal strain name: W22). SEQ ID NO: 3 represents the nucleotide sequence of the gene of glucan synthase II of S. commune strain Lu15531. SEQ ID NO: 5 represents the cDNA sequence of glucan synthase I of S. commune strain Lu15531. SEQ ID NO: 7 represents the cDNA sequence of glucan synthase II of S. commune strain Lu15531. SEQ ID NO: 9 represents the nucleotide sequence of the gene of glucan synthase I of S. commune strain Lu15634 (strain collection, BASF SE; monocaryotic strain originating from dicaryotic S. commune strain from strain collection at the Technical University of Braunschweig (Germany), Prof. Rau; generated by spore isolation). SEQ ID NO: 11 represents the nucleotide sequence of the gene of glucan synthase II of S. commune strain Lu15634. SEQ ID NO: 13 represents the cDNA sequence of glucan synthase I of S. commune strain Lu15634. SEQ ID NO: 15 represents the cDNA sequence of glucan synthase II of S. commune strain Lu15634.

The polypeptide as referred to and to be used in context with the present invention and the polypeptide encoded by the polynucleotide in context of the present invention (said polypeptides hereinafter also referred to as the “polypeptide in context of the present invention”) has 1,3-β-D-glucan synthase-activity. In one embodiment, it is a 1,3-β-D-glucan synthase. For example, the polypeptide in context of the present invention may comprise or consist of an amino acid sequence which at least 70%, preferably at least 75%, more preferably at least 80%, more preferably at least 85%, more preferably at least 90%, more preferably at least 95%, more preferably at least 96%, more preferably at least 97%, more preferably at least 98%, more preferably at least 99%, more preferably at least 99.5%, and most preferably 100% identical to SEQ ID NO: 6, 8, 14 or 16, provided that the polypeptide has 1,3-β-D-glucan synthase-activity. SEQ ID NO: 6 represents the amino acid sequence of glucan synthase I of S. commune strain Lu15531. SEQ ID NO: 8 represents the amino acid sequence of glucan synthase II of S. commune strain Lu15531. SEQ ID NO: 14 represents the amino acid sequence of glucan synthase I of S. commune strain Lu15634. SEQ ID NO: 16 represents the amino acid sequence of glucan synthase II of S. commune strain Lu15634.

In context with the present invention, the term “1,3-β-D-glucan synthase-activity” means that the respective polypeptide is capable of catalyzing the elongation of the 1,3-β-D-glucan chain (chain can be linear or branched) using UDP-glucose as substrate (see Inoue, Eur J Biochem (1995), 231: 845-854). For example, in context with the present invention, a polynucleotide may be considered to encode a polypeptide having 1,3-β-D-glucan synthase-activity if an S. commune cell which is transformed with said polynucleotide and which expresses said polynucleotide constitutively is able to produce at least 50%, more preferably at least 75%, more preferably at least 100%, more preferably at least 120%, more preferably at least 150%, more preferably at least 200%, and most preferably at least 220% more schizophyllan compared to an S. commune cell not being transformed with said polynucleotide, wherein the following conditions may be applied. The respective S. commune cultures with transformed and non-transformed cells, respectively, may be cultivated as follows. For the liquid cultures, the following medium may be used (hereinafter referred to as “Standard Medium”): 30 g glucose (Sigma), 3 g yeast extract (Difco), 1 g KH₂PO₄ (Riedel-de Haën), 0.5 g MgSO₄×7H₂O (Roth) per liter H₂O, For both, pre-cultures and for main culture, 250 ml shaking flasks filled with 30 ml Standard Medium may be used. The cultivation may be carried out at 27° C. and 225 rpm. Before each inoculation, the biomass may be homogenized for 1 minute at 13500 rpm using T 25 digital ULTRA-TURRAX® (IKA). The first pre-culture may be inoculated with 50 mg of wet biomass. The cultures may then be incubated for 72 hours. After 72 hours, the second pre-culture may be started. The concentration of the homogenized wet biomass from the first pre-culture used for inoculation may be 250 mg. Cultivation time may be 45 hours. After 45 hours, the main culture may be inoculated with 500 mg of homogenized wet biomass from the second pre-culture and cultivated for another 45 hours. Subsequently, the cultures may be treated as follows. 10 ml of the culture, 20 ml H₂O and 90 μl Acticide BW20 may be mixed. The sample may then be digested for 24 h at 40° C. with β-glucanase (0.3 ml) (Erbslöh). After the incubation, the sample may be centrifuged (e.g., 30 minutes at 3400 g) and the supernatant may be analyzed for glucose content using HPLC cation exchanger (Aminex HPX-87-H, BIO-RAD) with 0.5 M H₂SO₄ (Roth) as eluent and 0.5 ml/min flow rate at 30° C. The typical schizophyllan structure as described herein may be confirmed by further analytical approaches as described in the Example herein below (e.g., by NMR and XRD). The same evaluation may be performed mutatis mutandis for assessing whether a given polypeptide has 1,3-β-D-glucan synthase-activity in context of the present invention. In this case, a corresponding polynucleotide encoding said polypeptide to be assessed is evaluated mutatis mutandis as described above. If the expression of such a polynucleotide encoding said polypeptide to be assessed is considered to encode a polypeptide having 1,3-β-D-glucan synthase-activity as described above, the polypeptide itself is considered to have 1,3-β-D-glucan synthase-activity.

The level of identity between two or more sequences (e.g., nucleic acid sequences or amino acid sequences) can be easily determined by methods known in the art, e.g., by BLAST analysis. Generally, in context with the present invention, if two sequences (e.g., polynucleotide sequences or amino acid sequences) to be compared by, e.g., sequence comparisons differ in identity, then the term “identity” may refer to the shorter sequence and that part of the longer sequence that matches said shorter sequence. Therefore, when the sequences which are compared do not have the same length, the degree of identity may preferably either refer to the percentage of nucleotide residues in the shorter sequence which are identical to nucleotide residues in the longer sequence or to the percentage of nucleotides in the longer sequence which are identical to nucleotide sequence in the shorter sequence. In this context, the skilled person is readily in the position to determine that part of a longer sequence that matches the shorter sequence. Furthermore, as used herein, identity levels of nucleic acid sequences or amino acid sequences may refer to the entire length of the respective sequence and is preferably assessed pair-wise, wherein each gap is to be counted as one mismatch. These definitions for sequence comparisons (e.g., establishment of “identity” values) are to be applied for all sequences described and disclosed herein.

Moreover, the term “identity” as used herein means that there is a functional and/or structural equivalence between the corresponding sequences. Nucleic acid/amino acid sequences having the given identity levels to the herein-described particular nucleic acid/amino acid sequences may represent derivatives/variants of these sequences which, preferably, have the same biological function. They may be either naturally occurring variations, for instance sequences from other varieties, species, etc., or mutations, and said mutations may have formed naturally or may have been produced by deliberate mutagenesis. Furthermore, the variations may be synthetically produced sequences. The variants may be naturally occurring variants or synthetically produced variants or variants produced by recombinant DNA techniques. Deviations from the above-described nucleic acid sequences may have been produced, e.g., by deletion, substitution, addition, insertion and/or recombination. The term “addition” refers to adding at least one nucleic acid residue/amino acid to the end of the given sequence, whereas “insertion” refers to inserting at least one nucleic acid residue/amino acid within a given sequence. The term “deletion” refers to deleting or removal of at least one nucleic acid residue or amino acid residue in a given sequence. The term “substitution” refers to the replacement of at least one nucleic acid residue/amino acid residue in a given sequence. Again, these definitions as used here apply, mutatis mutandis, for all sequences provided and described herein.

Generally, as used herein, the terms “polynucleotide” and “nucleic acid” or “nucleic acid molecule” are to be construed synonymously. Generally, nucleic acid molecules may comprise inter alia DNA molecules, RNA molecules, oligonucleotide thiophosphates, substituted ribo-oligonucleotides or PNA molecules. Furthermore, the term “nucleic acid molecule” may refer to DNA or RNA or hybrids thereof or any modification thereof that is known in the art (see, e.g., U.S. Pat. No. 5,525,711, U.S. Pat. No. 4,711,955, U.S. Pat. No. 5,792,608 or EP 302175 for examples of modifications). The polynucleotide sequence may be single- or double-stranded, linear or circular, natural or synthetic, and without any size limitation. For instance, the polynucleotide sequence may be genomic DNA, cDNA, mitochondrial DNA, mRNA, antisense RNA, ribozymal RNA or a DNA encoding such RNAs or chimeroplasts (Gamper, Nucleic Acids Research, 2000, 28, 4332-4339). Said polynucleotide sequence may be in the form of a vector, plasmid or of viral DNA or RNA. Also described herein are nucleic acid molecules which are complementary to the nucleic acid molecules described above and nucleic acid molecules which are able to hybridize to nucleic acid molecules described herein. A nucleic acid molecule described herein may also be a fragment of the nucleic acid molecules in context of the present invention. Particularly, such a fragment is a functional fragment. Examples for such functional fragments are nucleic acid molecules which can serve as primers.

The term “hybridization” or “hybridizes” as used herein in context of nucleic acid molecules/DNA sequences may relate to hybridizations under stringent or non-stringent conditions. If not further specified, the conditions are preferably non-stringent. Said hybridization conditions may be established according to conventional protocols described, for example, in Sambrook, Russell “Molecular Cloning, A Laboratory Manual”, Cold Spring Harbor Laboratory, N. Y. (2001); Current Protocols in Molecular Biology, Update May 9, 2012, Print ISSN: 1934-3639, Online ISSN: 1934-3647; Ausubel, “Current Protocols in Molecular Biology”, Green Publishing Associates and Wiley Interscience, N. Y. (1989), or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as 0.1×SSC, 0.1% SDS at 65° C. Non-stringent hybridization conditions for the detection of homologous or not exactly complementary sequences may be set at 6×SSC, 1% SDS at 65° C. As is well known, the length of the probe and the composition of the nucleic acid to be determined constitute further parameters of the hybridization conditions. Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. In accordance to the invention described herein, low stringent hybridization conditions for the detection of homologous or not exactly complementary sequences may, for example, be set at 6×SSC, 1% SDS at 65° C. As is well known, the length of the probe and the composition of the nucleic acid to be determined constitute further parameters of the hybridization conditions.

Hybridizing nucleic acid molecules also comprise fragments of the above described molecules. Such fragments may represent nucleic acid molecules which code for a functional 1,3-β-D-glucan synthase as described herein or a functional fragment thereof which can serve as a primer. Furthermore, nucleic acid molecules which hybridize with any of the aforementioned nucleic acid molecules also include complementary fragments, derivatives and variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed). The terms complementary or complementarity refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single-stranded molecules. The degree of complementarity between nucleic acid strands has significant effects on the efficiency and strength of hybridization between nucleic acid strands. This is of particular importance in amplification reactions, which depend upon binding between nucleic acids strands. The term “hybridizing sequences” preferably refers to sequences which display a sequence identity of at least 45%, more preferably at least 50%, more preferably at least 55%, more preferably at least 60%, more preferably at least 65%, more preferably at least 70%, more preferably at least 75%, more preferably at least 80%. more preferably at least 85%, more preferably at least 90%, more preferably at least 95%, more preferably at least 96%, more preferably at least 97%, more preferably at least 98% more preferably at least 99%, more preferably at least 99.5%, and most preferably 100% identity with a nucleic acid sequence as described herein encoding a 1,3-β-D-glucan synthase.

Also described herein are vectors containing a polynucleotide in context of the present invention. The present invention relates also to a vector comprising the polynucleotide in context of the present invention. The term “vector” as used herein particularly refers to plasmids, cosmids, viruses, bacteriophages and other vectors commonly used in genetic engineering. In a preferred embodiment, the vectors are suitable for the transformation, transduction and/or transfection of microorganisms as described herein, e.g., fungal cells, prokaryotic ells (e.g., bacteria), yeast, and the like. Specific examples of microorganisms in context with the present invention are Schizophyllum commune, Sclerotium rolfsii, Sclerotium glucanicum, Sclerotium delphinii, Porodisculus pendulus, Botrytis cinema, Laminaria sp., Lentinula edoles, and Monilinia fructigena. In a particularly preferred embodiment, said vectors are suitable for stable transformation of the microorganism, for example to express the polypeptide having 1,3-β-D-glucan synthase activity as described herein.

Accordingly, in one aspect of the invention, the vector as provided is an expression vector. Generally, expression vectors have been widely described in the literature. As a rule, they may not only contain a selection marker gene and a replication-origin ensuring replication in the host selected, but also a promoter, and in most cases a termination signal for transcription. Between the promoter and the termination signal there is preferably at least one restriction site or a polylinker which enables the insertion of a nucleic acid sequence/molecule desired to be expressed.

It is to be understood that when the vector provided herein is generated by taking advantage of an expression vector known in the prior art that already comprises a promoter suitable to be employed in context of this invention, for example expression of a polypeptide having 1,3-β-D-glucan synthase activity as described herein. The nucleic acid construct is preferably inserted into that vector in a manner the resulting vector comprises only one promoter suitable to be employed in context of this invention. The skilled person knows how such insertion can be put into practice. For example, the promoter can be excised either from the nucleic acid construct or from the expression vector prior to ligation. A non-limiting example of the vector of the present invention is pBluescript II comprising the polynucleotide in context of the present invention. Further examples of vectors suitable to comprise the polynucleotide in context of the present invention to form the described herein are known in the art and comprise, for example pDrive, pTOPO, pUC19 and pUC21.

Generally, the present invention relates to all the embodiments described herein as well as to all permutations and combinations thereof. The following particular aspects of the present invention must not be construed as limiting the scope of the present invention on such aspects.

In one aspect, the present invention relates to a genetically modified microorganism capable of producing schizophyllan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain.

In one aspect, the present invention relates to a genetically modified microorganism capable of producing schizophyllan, characterized in that said genetically modified microorganism contains at least one copy more of a polynucleotide encoding a polypeptide having 1,3-β-D-glucan synthase-activity compared to a corresponding non-modified control microorganism of the same strain.

In another aspect, the present invention relates to a genetically modified microorganism of the species Schizoyphyllum commune, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain.

In another aspect, the present invention relates to a genetically modified microorganism of the species Schizoyphyllum commune, characterized in that said genetically modified microorganism contains at least one copy more of a polynucleotide encoding a polypeptide having 1,3-β-D-glucan synthase-activity compared to a corresponding non-modified control microorganism of the same strain.

In one aspect, the present invention relates to a genetically modified microorganism capable of producing scleroglucan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain.

In another aspect, the present invention relates to a genetically modified microorganism capable of producing scleroglucan, characterized in that said genetically modified microorganism contains at least one copy more of a polynucleotide encoding a polypeptide having 1,3-β-D-glucan synthase-activity compared to a corresponding non-modified control microorganism of the same strain.

In another aspect, the present invention relates to a genetically modified microorganism of the species Sclerotium rolfsii, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain.

In another aspect, the present invention relates to a genetically modified microorganism of the species Sclerotium rolfsii, characterized in that said genetically modified microorganism contains at least one copy more of a polynucleotide encoding a polypeptide having 1,3-β-D-glucan synthase-activity compared to a corresponding non-modified control microorganism of the same strain.

In another aspect, the present invention relates to a genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15.

In another aspect, the present invention relates to a genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15.

In another aspect, the present invention relates to a genetically modified microorganism capable of producing schizophyllan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15.

In another aspect, the present invention relates to a genetically modified microorganism capable of producing schizophyllan, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15.

In another aspect, the present invention relates to a genetically modified microorganism of the species Schizoyphyllum commune, characterized in that said genetically modified microorganism overexpresses (i) a polynucleotide encoding a polypeptide having 1,3-β-D-glucan synthase-activity, and/or (ii) a polypeptide having synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15.

In another aspect, the present invention relates to a genetically modified microorganism of the species Schizoyphyllum commune, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15.

In another aspect, the present invention relates to a genetically modified microorganism capable of producing scleroglucan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15.

In another aspect, the present invention relates to a genetically modified microorganism capable of producing scleroglucan, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15.

In another aspect, the present invention relates to a genetically modified microorganism of the species Sclerotium rolfsii, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15.

In another aspect, the present invention relates to a genetically modified microorganism of the species Sclerotium rolfsii, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15.

In another aspect, the present invention relates to a genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16.

In another aspect, the present invention relates to a genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism contains at least one copy more of a polynucleotide encoding a polypeptide having 1,3-β-D-glucan synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16.

In another aspect, the present invention relates to a genetically modified microorganism capable of producing schizophyllan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16.

In another aspect, the present invention relates to a genetically modified microorganism capable of producing schizophyllan, characterized in that said genetically modified microorganism contains at least one copy more of a polynucleotide encoding a polypeptide having 1,3-β-D-glucan synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16.

In another aspect, the present invention relates to a genetically modified microorganism of the species Schizoyphyllum commune, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16.

In another aspect, the present invention relates to a genetically modified microorganism of the species Schizoyphyllum commune, characterized in that said genetically modified microorganism contains at least one copy more of a polynucleotide encoding a polypeptide having synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16.

In another aspect, the present invention relates to a genetically modified microorganism capable of producing scleroglucan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16.

In another aspect, the present invention relates to a genetically modified microorganism capable of producing scleroglucan, characterized in that said genetically modified microorganism contains at least one copy more of a polynucleotide encoding a polypeptide having 1,3-β-D-glucan synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16.

In another aspect, the present invention relates to a genetically modified microorganism of the species Sclerotium rolfsii, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16.

In another aspect, the present invention relates to a genetically modified microorganism of the species Sclerotium rolfsii, characterized in that said genetically modified microorganism contains at least one copy more of a polynucleotide encoding a polypeptide having 1,3-β-D-glucan synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16.

In another aspect, the present invention relates to the use of a polynucleotide encoding a polypeptide having 1,3-β-D-glucan synthase-activity for producing schizophyllan.

In another aspect, the present invention relates to the use of a polynucleotide encoding a polypeptide having 1,3-β-D-glucan synthase-activity for producing scleroglucan.

In another aspect, the present invention relates to the use of a polynucleotide encoding a polypeptide having 1,3-β-D-glucan synthase-activity for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15.

In another aspect, the present invention relates to the use of a polynucleotide encoding a polypeptide having 1,3-β-D-glucan synthase-activity for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, wherein said polypeptide is at least 70%, 75%, 80%, 85%, 90%, 95%, 96%, 97%, 98%, 99%, 99.5%, or 100% identical to SEQ ID NO: 6, 8, 14 or 16.

In another aspect, the present invention relates to the use of a polynucleotide encoding a polypeptide having 1,3-β-D-glucan synthase-activity for producing schizophyllan, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15.

In another aspect, the present invention relates to the use of a polynucleotide encoding a polypeptide having 1,3-β-D-glucan synthase-activity for producing schizophyllan, wherein said polypeptide is at least 70%, 75%, 80%, 85%, 90%, 95%, 96%, 97%, 98%, 99%, 99.5%, or 100% identical to SEQ ID NO: 6, 8, 14 or 16.

In another aspect, the present invention relates to the use of a polynucleotide encoding a polypeptide having 1,3-β-D-glucan synthase-activity for producing scleroglucan, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15.

In another aspect, the present invention relates to the use of a polynucleotide encoding a polypeptide having 1,3-β-D-glucan synthase-activity for producing scleroglucan, wherein said polypeptide is at least 70%, 75%, 80%, 85%, 90%, 95%, 96%, 97%, 98%, 99%, 99.5%, or 100% identical to SEQ ID NO: 6, 8, 14 or 16.

In another aspect, the present invention relates to the use of a polypeptide having 1,3-β-D-glucan synthase-activity for producing schizophyllan.

In another aspect, the present invention relates to the use of a polypeptide having 1,3-β-D-glucan synthase-activity for producing scleroglucan.

In another aspect, the present invention relates to the use of polypeptide having 1,3-β-D-glucan synthase-activity for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, wherein said polypeptide is at least 70%, 75%, 80%, 85%, 90%, 95%, 96%, 97%, 98%, 99%, 99.5%, or 100% identical to SEQ ID NO: 6, 8, 14 or 16.

In another aspect, the present invention relates to the use of a polypeptide having 1,3-β-D-glucan synthase-activity for producing schizophyllan, wherein said polypeptide is at least 70%, 75%, 80%, 85%, 90%, 95%, 96%, 97%, 98%, 99%, 99.5%, or 100% identical to SEQ ID NO: 6, 8, 14 or 16.

In another aspect, the present invention relates to the use of polypeptide having 1,3-β-D-glucan synthase-activity for producing scleroglucan, wherein said polypeptide is at least 70%, 75%, 80%, 85%, 90%, 95%, 96%, 97%, 98%, 99%, 99.5%, or 100% identical to SEQ ID NO: 6, 8, 14 or 16.

In another aspect, the present invention relates to the use of a genetically modified microorganism capable of producing schizophyllan, wherein said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism capable of producing schizophyllan, wherein said genetically modified microorganism contains at least one copy more of a polynucleotide encoding a polypeptide having 1,3-β-D-glucan synthase-activity compared to a corresponding non-modified control microorganism of the same strain, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism capable of producing schizophyllan, wherein said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, for producing schizophyllan.

In another aspect, the present invention relates to the use of a genetically modified microorganism capable of producing schizophyllan, wherein said genetically modified microorganism contains at least one copy more of a polynucleotide encoding a polypeptide having 1,3-β-D-glucan synthase-activity compared to a corresponding non-modified control microorganism of the same strain, for producing schizophyllan.

In another aspect, the present invention relates to the use of a genetically modified microorganism capable of producing schizophyllan, wherein said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, for producing scleroglucan.

In another aspect, the present invention relates to the use of a genetically modified microorganism capable of producing schizophyllan, wherein said genetically modified microorganism contains at least one copy more of a polynucleotide encoding a polypeptide having 1,3-β-D-glucan synthase-activity compared to a corresponding non-modified control microorganism of the same strain, for producing scleroglucan. In another aspect, the present invention relates to the use of a genetically modified microorganism of the species Schizoyphyllum commune, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism of the species Schizoyphyllum commune, characterized in that said genetically modified microorganism contains at least one copy more of a polynucleotide encoding a polypeptide having 1,3-β-D-glucan synthase-activity compared to a corresponding non-modified control microorganism of the same strain, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism of the species Schizoyphyllum commune, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, for producing schizophyllan.

In another aspect, the present invention relates to the use of a genetically modified microorganism of the species Schizoyphyllum commune, characterized in that said genetically modified microorganism contains at least one copy more of a polynucleotide encoding a polypeptide having 1,3-β-D-glucan synthase-activity compared to a corresponding non-modified control microorganism of the same strain, for producing schizophyllan.

In another aspect, the present invention relates to the use of a genetically modified microorganism of the species Schizoyphyllum commune, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, for producing scleroglucan.

In another aspect, the present invention relates to the use of a genetically modified microorganism of the species Schizoyphyllum commune, characterized in that said genetically modified microorganism contains at least one copy more of a polynucleotide encoding a polypeptide having 1,3-β-D-glucan synthase-activity compared to a corresponding non-modified control microorganism of the same strain, for producing scleroglucan.

In another aspect, the present invention relates to the use of a genetically modified microorganism capable of producing scleroglucan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism capable of producing scleroglucan, characterized in that said genetically modified microorganism contains at least one copy more of a polynucleotide encoding a polypeptide having 1,3-β-D-glucan synthase-activity compared to a corresponding non-modified control microorganism of the same strain, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism capable of producing scleroglucan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, for producing schizophyllan.

In another aspect, the present invention relates to the use of a genetically modified microorganism capable of producing scleroglucan, characterized in that said genetically modified microorganism contains at least one copy more of a polynucleotide encoding a polypeptide having 1,3-β-D-glucan synthase-activity compared to a corresponding non-modified control microorganism of the same strain, for producing schizophyllan.

In another aspect, the present invention relates to the use of a genetically modified microorganism capable of producing scleroglucan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, for producing scleroglucan.

In another aspect, the present invention relates to the use of a genetically modified microorganism capable of producing scleroglucan, characterized in that said genetically modified microorganism contains at least one copy more of a polynucleotide encoding a polypeptide having 1,3-β-D-glucan synthase-activity compared to a corresponding non-modified control microorganism of the same strain, for producing scleroglucan.

In another aspect, the present invention relates to the use of a genetically modified microorganism of the species Sclerotium rolfsii, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism of the species Sclerotium rolfsii, characterized in that said genetically modified microorganism contains at least one copy more of a polynucleotide encoding a polypeptide having 1,3-β-D-glucan synthase-activity compared to a corresponding non-modified control microorganism of the same strain, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism of the species Sclerotium rolfsii, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, for schizophyllan.

In another aspect, the present invention relates to the use of a genetically modified microorganism of the species Sclerotium rolfsii, characterized in that said genetically modified microorganism contains at least one copy more of a polynucleotide encoding a polypeptide having 1,3-β-D-glucan synthase-activity compared to a corresponding non-modified control microorganism of the same strain, for schizophyllan.

In another aspect, the present invention relates to the use of a genetically modified microorganism of the species Sclerotium rolfsii, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, for scleroglucan.

In another aspect, the present invention relates to the use of a genetically modified microorganism of the species Sclerotium rolfsii, characterized in that said genetically modified microorganism contains at least one copy more of a polynucleotide encoding a polypeptide having 1,3-β-D-glucan synthase-activity compared to a corresponding non-modified control microorganism of the same strain, for scleroglucan.

In another aspect, the present invention relates to the use of a genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing schizophyllan.

In another aspect, the present invention relates to the use of a genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing schizophyllan.

In another aspect, the present invention relates to the use of a genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing scleroglucan.

In another aspect, the present invention relates to the use of a genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing scleroglucan.

In another aspect, the present invention relates to the use of a genetically modified microorganism capable of producing schizophyllan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism capable of producing schizophyllan, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism capable of producing schizophyllan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing schizophyllan.

In another aspect, the present invention relates to the use of a genetically modified microorganism capable of producing schizophyllan, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing schizophyllan.

In another aspect, the present invention relates to the use of a genetically modified microorganism capable of producing schizophyllan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing scleroglucan.

In another aspect, the present invention relates to the use of a genetically modified microorganism capable of producing schizophyllan, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing scleroglucan.

In another aspect, the present invention relates to the use of a genetically modified microorganism of the species Schizoyphyllum commune, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism of the species Schizoyphyllum commune, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism of the species Schizoyphyllum commune, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing schizophyllan.

In another aspect, the present invention relates to the use of a genetically modified microorganism of the species Schizoyphyllum commune, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing schizophyllan.

In another aspect, the present invention relates to the use of a genetically modified microorganism of the species Schizoyphyllum commune, characterized in that said genetically modified microorganism overexpresses (i) a polynucleotide encoding a polypeptide having 1,3-β-D-glucan synthase-activity, and/or (ii) a polypeptide having 1,3-synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing scleroglucan.

In another aspect, the present invention relates to the use of a genetically modified microorganism of the species Schizoyphyllum commune, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing scleroglucan.

In another aspect, the present invention relates to the use of a genetically modified microorganism capable of producing scleroglucan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism capable of producing scleroglucan, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing a polymer consisting of a linear main chain of β-D-(1-3)-glucopyranosyl units having a single ii-D-glucopyranosyl unit (1-6) linked to a β-D-glucopyranosyl unit of the linear main chain with an average branching degree of about 0.3.

In another aspect, the present invention relates to a genetically modified microorganism capable of producing scleroglucan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing schizophyllan.

In another aspect, the present invention relates to a genetically modified microorganism capable of producing scleroglucan, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing schizophyllan.

In another aspect, the present invention relates to the use of a genetically modified microorganism capable of producing scleroglucan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing scleroglucan.

In another aspect, the present invention relates to the use of a genetically modified microorganism capable of producing scleroglucan, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing scleroglucan.

In another aspect, the present invention relates to the use of a genetically modified microorganism of the species Sclerotium rolfsii, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism of the species Sclerotium rolfsii, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism of the species Sclerotium rolfsii, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing schizophyllan.

In another aspect, the present invention relates to the use of a genetically modified microorganism of the species Sclerotium rolfsii, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing schizophyllan.

In another aspect, the present invention relates to the use of a genetically modified microorganism of the species Sclerotium rolfsii, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing scleroglucan.

In another aspect, the present invention relates to the use of a genetically modified microorganism of the species Sclerotium rolfsii, characterized in that said genetically modified microorganism contains at least one copy more of a polynucleotide encoding a polypeptide having 1,3-β-D-glucan synthase-activity compared to a corresponding non-modified control microorganism of the same strain, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15, for producing scleroglucan.

In another aspect, the present invention relates to the use of a genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism contains at least one copy more of a polynucleotide encoding a polypeptide having 1,3-β-D-glucan synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing schizophyllan.

In another aspect, the present invention relates to the use of a genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism contains at least one copy more of a polynucleotide encoding a polypeptide having 1,3-β-D-glucan synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing schizophyllan.

In another aspect, the present invention relates to the use of a genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing scleroglucan.

In another aspect, the present invention relates to the use of a genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism contains at least one copy more of a polynucleotide encoding a polypeptide having 1,3-β-D-glucan synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing scleroglucan.

In another aspect, the present invention relates to the use of a genetically modified microorganism capable of producing schizophyllan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism capable of producing schizophyllan, characterized in that said genetically modified microorganism contains at least one copy more of a polynucleotide encoding a polypeptide having 1,3-β-D-glucan synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism capable of producing schizophyllan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing schizophyllan.

In another aspect, the present invention relates to the use of a genetically modified microorganism capable of producing schizophyllan, characterized in that said genetically modified microorganism contains at least one copy more of a polynucleotide encoding a polypeptide having 1,3-β-D-glucan synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing schizophyllan.

In another aspect, the present invention relates to the use of a genetically modified microorganism capable of producing schizophyllan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing scleroglucan.

In another aspect, the present invention relates to the use of a genetically modified microorganism capable of producing schizophyllan, characterized in that said genetically modified microorganism contains at least one copy more of a polynucleotide encoding a polypeptide having 1,3-β-D-glucan synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing scleroglucan.

In another aspect, the present invention relates to the use of a genetically modified microorganism of the species Schizoyphyllum commune, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism of the species Schizoyphyllum commune, characterized in that said genetically modified microorganism contains at least one copy more of a polynucleotide encoding a polypeptide having 1,3-β-D-glucan synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism of the species Schizoyphyllum commune, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing schizophyllan.

In another aspect, the present invention relates to the use of a genetically modified microorganism of the species Schizoyphyllum commune, characterized in that said genetically modified microorganism contains at least one copy more of a polynucleotide encoding a polypeptide having 1,3-β-D-glucan synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing schizophyllan.

In another aspect, the present invention relates to the use of a genetically modified microorganism of the species Schizoyphyllum commune, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing scleroglucan.

In another aspect, the present invention relates to the use of a genetically modified microorganism of the species Schizoyphyllum commune, characterized in that said genetically modified microorganism contains at least one copy more of a polynucleotide encoding a polypeptide having 1,3-β-D-glucan synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing scleroglucan.

In another aspect, the present invention relates to the use of a genetically modified microorganism capable of producing scleroglucan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism capable of producing scleroglucan, characterized in that said genetically modified microorganism contains at least one copy more of a polynucleotide encoding a polypeptide having 1,3-β-D-glucan synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism capable of producing scleroglucan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing schizophyllan.

In another aspect, the present invention relates to the use of a genetically modified microorganism capable of producing scleroglucan, characterized in that said genetically modified microorganism contains at least one copy more of a polynucleotide encoding a polypeptide having 1,3-β-D-glucan synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing schizophyllan.

In another aspect, the present invention relates to the use of a genetically modified microorganism capable of producing scleroglucan, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing scleroglucan.

In another aspect, the present invention relates to the use of a genetically modified microorganism capable of producing scleroglucan, characterized in that said genetically modified microorganism contains at least one copy more of a polynucleotide encoding a polypeptide having 1,3-β-D-glucan synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing scleroglucan.

In another aspect, the present invention relates to the use of a genetically modified microorganism of the species Sclerotium rolfsii, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism of the species Sclerotium rolfsii, characterized in that said genetically modified microorganism contains at least one copy more of a polynucleotide encoding a polypeptide having 1,3-β-D-glucan synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing a polymer consisting of a linear main chain of 3-D-(1-3)-glucopyranosyl units having a single β-D-glucopyranosyl unit (1-6) linked to a β-D-glucopyranosyl unit of the linear main chain with an average branching degree of about 0.3.

In another aspect, the present invention relates to the use of a genetically modified microorganism of the species Sclerotium rolfsii, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing schizophyllan.

In another aspect, the present invention relates to the use of a genetically modified microorganism of the species Sclerotium rolfsii, characterized in that said genetically modified microorganism contains at least one copy more of a polynucleotide encoding a polypeptide having 1,3-β-D-glucan synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing schizophyllan.

In another aspect, the present invention relates to the use of a genetically modified microorganism of the species Sclerotium rolfsii, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing scleroglucan.

In another aspect, the present invention relates to the use of a genetically modified microorganism of the species Sclerotium rolfsii, characterized in that said genetically modified microorganism contains at least one copy more of a polynucleotide encoding a polypeptide having 1,3-β-D-glucan synthase-activity compared to a corresponding non-modified control microorganism of the same strain, wherein said polypeptide is at least 70%, 75%, 80%, 85%, 90%, 95%, 96%, 97%, 98%, 99%, 99.5%, or 100% identical to SEQ ID NO: 6, 8, 14 or 16, for producing scleroglucan.

In another aspect, the present invention relates to a method of producing schizophyllan, said method comprising the steps of:
(a) introducing a strong (e.g., constitutive or inducible) promoter upstream of a polynucleotide encoding a polypeptide having 1,3-β-D-glucan synthase-activity thereby increasing the expression of said polynucleotide, or a polynucleotide encoding a polypeptide having 1,3-β-D-glucan synthase-activity into a microorganism being able to synthesize schizophyllan;
(b) culturing said microorganism of (a) in a medium, thereby allowing said microorganism to produce schizophyllan; and
(c) optionally recovering schizophyllan from the medium.

In another aspect, the present invention relates to a method of producing scleroglucan, said method comprising the steps of:
(a) introducing a strong (e.g., constitutive or inducible) promoter upstream of a polynucleotide encoding a polypeptide having 1,3-β-D-glucan synthase-activity thereby increasing the expression of said polynucleotide, or a polynucleotide encoding a polypeptide having 1,3-β-D-glucan synthase-activity into a microorganism being able to synthesize scleroglucan;
(b) culturing said microorganism of (a) in a medium, thereby allowing said microorganism to produce scleroglucan; and
(c) optionally recovering scleroglucan from the medium.

In another aspect, the present invention relates to a method of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, said method comprising the steps of:
(a) introducing a strong (e.g., constitutive or inducible) promoter upstream of a polynucleotide encoding a polypeptide having 1,3-β-D-glucan synthase-activity thereby increasing the expression of said polynucleotide, or a polynucleotide encoding a polypeptide having synthase-activity into a microorganism of the species Schizophyllum commune being able to synthesize said polymer;
(b) culturing said microorganism of (a) in a medium, thereby allowing said microorganism to produce said polymer; and
(c) optionally recovering said polymer from the medium.

In another aspect, the present invention relates to a method of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, said method comprising the steps of:
(a) introducing a strong (e.g., constitutive or inducible) promoter upstream of a polynucleotide encoding a polypeptide having 1,3-β-D-glucan synthase-activity thereby increasing the expression of said polynucleotide, or a polynucleotide encoding a polypeptide having 1,3-β-D-glucan synthase-activity into a microorganism of the species Sclerotium rolfsii being able to synthesize said polymer;
(b) culturing said microorganism of (a) in a medium, thereby allowing said microorganism to produce said polymer; and
(c) optionally recovering said polymer from the medium.

In another aspect, the present invention relates to a method of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, said method comprising the steps of:
(a) introducing a polynucleotide encoding a polypeptide having 1,3-β-D-glucan synthase-activity into a microorganism being able to synthesize said polymer, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15;
(b) culturing said microorganism of (a) in a medium, thereby allowing said microorganism to produce said polymer; and
(c) optionally recovering said polymer from the medium.

In another aspect, the present invention relates to a method of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, said method comprising the steps of:
(a) introducing a polynucleotide encoding a polypeptide having 1,3-β-D-glucan synthase-activity into a microorganism being able to synthesize said polymer, wherein said polypeptide is at least 70%, 75%, 80%, 85%, 90%, 95%, 96%, 97%, 98%, 99%, 99.5%, or 100% identical to SEQ ID NO: 6, 8, 14 or 16;
(b) culturing said microorganism of (a) in a medium, thereby allowing said microorganism to produce said polymer; and
(c) optionally recovering said polymer from the medium.

In another aspect, the present invention relates to a method of producing schizophyllan, said method comprising the steps of:
(a) introducing a polynucleotide encoding a polypeptide having 1,3-β-D-glucan synthase-activity into a microorganism being able to synthesize schizophyllan, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15;
(b) culturing said microorganism of (a) in a medium, thereby allowing said microorganism to produce schizophyllan; and
(c) optionally recovering schizophyllan from the medium.

In another aspect, the present invention relates to a method of producing scleroglucan, said method comprising the steps of:
(a) introducing a polynucleotide encoding a polypeptide having 1,3-β-D-glucan synthase-activity into a microorganism being able to synthesize scleroglucan, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15;
(b) culturing said microorganism of (a) in a medium, thereby allowing said microorganism to produce scleroglucan; and
(c) optionally recovering scleroglucan from the medium.

In another aspect, the present invention relates to a method of producing schizophyllan, said method comprising the steps of:
(a) introducing a polynucleotide encoding a polypeptide having 1,3-β-D-glucan synthase-activity into a microorganism being able to synthesize schizophyllan, wherein said polypeptide is at least 70%, 75%, 80%, 85%, 90%, 95%, 96%, 97%, 98%, 99%, 99.5%, or 100% identical to SEQ ID NO: 6, 8, 14 or 16;
(b) culturing said microorganism of (a) in a medium, thereby allowing said microorganism to produce schizophyllan; and
(c) optionally recovering schizophyllan from the medium.

In another aspect, the present invention relates to a method of producing scleroglucan, said method comprising the steps of:
(a) introducing a polynucleotide encoding a polypeptide having 1,3-β-D-glucan synthase-activity into a microorganism being able to synthesize scleroglucan, wherein said polypeptide is at least 70%, 75%, 80%, 85%, 90%, 95%, 96%, 97%, 98%, 99%, 99.5%, or 100% identical to SEQ ID NO: 6, 8, 14 or 16;
(b) culturing said microorganism of (a) in a medium, thereby allowing said microorganism to produce schizophyllan; and
(c) optionally recovering scleroglucan from the medium.

In another aspect, the present invention relates to a method of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, said method comprising the steps of:
(a) introducing a polynucleotide encoding a polypeptide having 1,3-β-D-glucan synthase-activity into a microorganism of the species Schizophyllum commune being able to synthesize said polymer, wherein said polypeptide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15;
(b) culturing said microorganism of (a) in a medium, thereby allowing said microorganism to produce said polymer; and
(c) optionally recovering said polymer from the medium.

In another aspect, the present invention relates to a method of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, said method comprising the steps of:
(a) introducing a polynucleotide encoding a polypeptide having 1,3-β-D-glucan synthase-activity into a microorganism of the species Sclerotium rolfsii being able to synthesize said polymer, wherein said polypeptide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15;
(b) culturing said microorganism of (a) in a medium, thereby allowing said microorganism to produce said polymer; and
(c) optionally recovering said polymer from the medium.

In another aspect, the present invention relates to a method of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, said method comprising the steps of:
(a) introducing a polynucleotide encoding a polypeptide having 1,3-β-D-glucan synthase-activity into a microorganism of the species Schizophyllum commune being able to synthesize said polymer, wherein said polypeptide is at least 70%, 75%, 80%, 85%, 90%, 95%, 96%, 97%, 98%, 99%, 99.5%, or 100% identical to SEQ ID NO: 6, 8, 14 or 16;
(b) culturing said microorganism of (a) in a medium, thereby allowing said microorganism to produce said polymer; and
(c) optionally recovering said polymer from the medium.

In another aspect, the present invention relates to a method of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, said method comprising the steps of:
(a) introducing a polynucleotide encoding a polypeptide having 1,3-β-D-glucan synthase-activity into a microorganism of the species Sclerotium rolfsii being able to synthesize said polymer, wherein said polypeptide is at least 70%, 75%, 80%, 85%, 90%, 95%, 96%, 97%, 98%, 99%, 99.5%, or 100% identical to SEQ ID NO: 6, 8, 14 or 16;
(b) culturing said microorganism of (a) in a medium, thereby allowing said microorganism to produce said polymer; and
(c) optionally recovering said polymer from the medium.

In another aspect, the present invention relates to a method of producing schizophyllan, said method comprising the steps of:
(a) introducing a polynucleotide encoding a polypeptide having 1,3-β-D-glucan synthase-activity into a microorganism of the species Schizophyllum commune being able to synthesize said polymer, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15;
(b) culturing said microorganism of (a) in a medium, thereby allowing said microorganism to produce said polymer; and
(c) optionally recovering said polymer from the medium.

In another aspect, the present invention relates to a method of producing scleroglucan, said method comprising the steps of:
(a) introducing a polynucleotide encoding a polypeptide having 1,3-β-D-glucan synthase-activity into a microorganism of the species Sclerotium rolfsii being able to synthesize said polymer, wherein said polynucleotide is at least 70%, 75%, 80%, 85%, 90%, 95%, 96%, 97%, 98%, 99%, 99.5%, or 100% identical to SEQ ID NO: 1, 3, 5, 7, 9, 11, 13 or 15;
(b) culturing said microorganism of (a) in a medium, thereby allowing said microorganism to produce said polymer; and
(c) optionally recovering said polymer from the medium.

In another aspect, the present invention relates to a method of producing schizophyllan, said method comprising the steps of:
(a) introducing a polynucleotide encoding a polypeptide having 1,3-β-D-glucan synthase-activity into a microorganism of the species Schizophyllum commune being able to synthesize said polymer, wherein said polypeptide is at least 70%, 75%, 80%, 85%, 90%, 95%, 96%, 97%, 98%, 99%, 99.5%, or 100% identical to SEQ ID NO: 6, 8, 14 or 16;
(b) culturing said microorganism of (a) in a medium, thereby allowing said microorganism to produce said polymer; and
(c) optionally recovering said polymer from the medium.

In another aspect, the present invention relates to a method of producing scleroglucan, said method comprising the steps of:
(a) introducing a polynucleotide encoding a polypeptide having 1,3-β-D-glucan synthase-activity into a microorganism of the species Sclerotium rolfsii being able to synthesize said polymer, wherein said polypeptide is at least 70%, 75%, 80%, 85%, 90%, 95%, 96%, 97%, 98%, 99%, 99.5%, or 100% identical to SEQ ID NO: 6, 8, 14 or 16;
(b) culturing said microorganism of (a) in a medium, thereby allowing said microorganism to produce said polymer; and
(c) optionally recovering said polymer from the medium.

The Figures show:

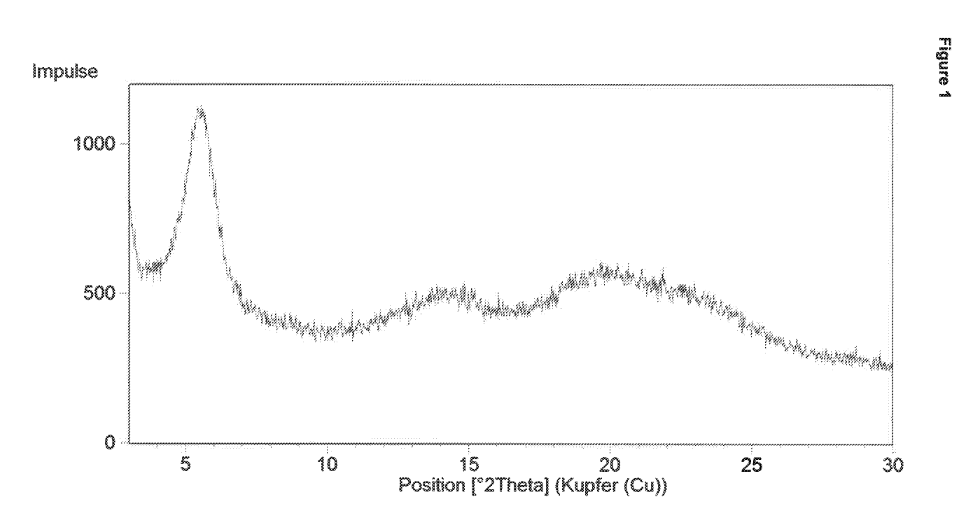

FIG. 1 XRD Spectrum of Schizophyllan sample. The triple helix could be seen as an intensive diffraction at 5° 2θ and the amorphous region of the material gives broad diffraction in the range of 20-25° 2θ

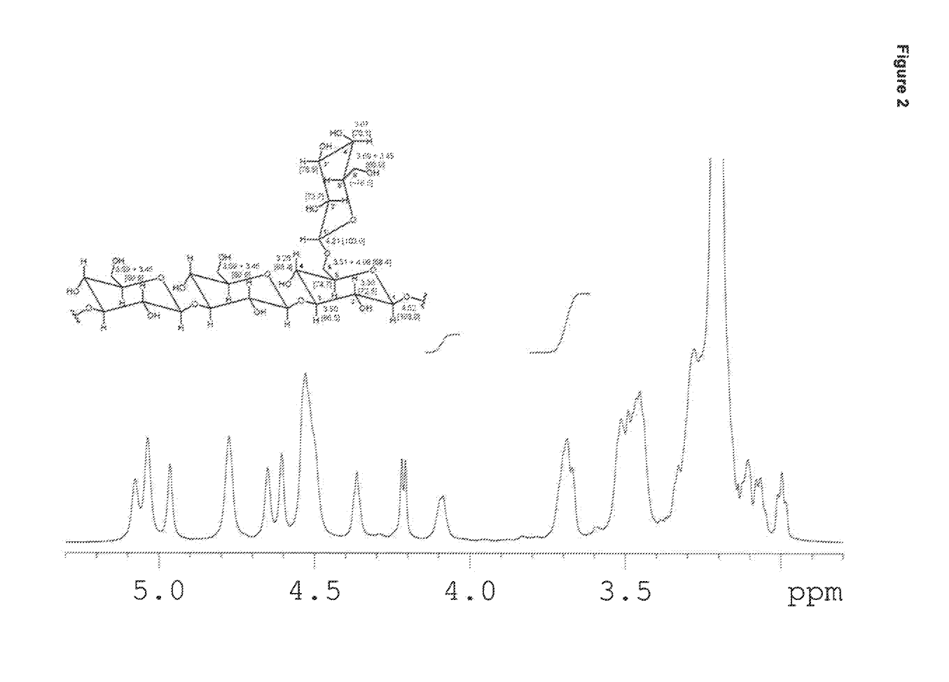

FIG. 2 ¹H-NMR of schizophyllan (50 mg of gel) in [D₆]-DMSO measured at 50° C. (16 scans, 600 MHz), The substitution pattern of schizophyllan can be assigned from the integrations of the CH₂OH at 3.7 ppm and CH₂O (ether) at 4.1 ppm signals, the ratio was determined to be 3.3:1 indicating the correct repeating unit.

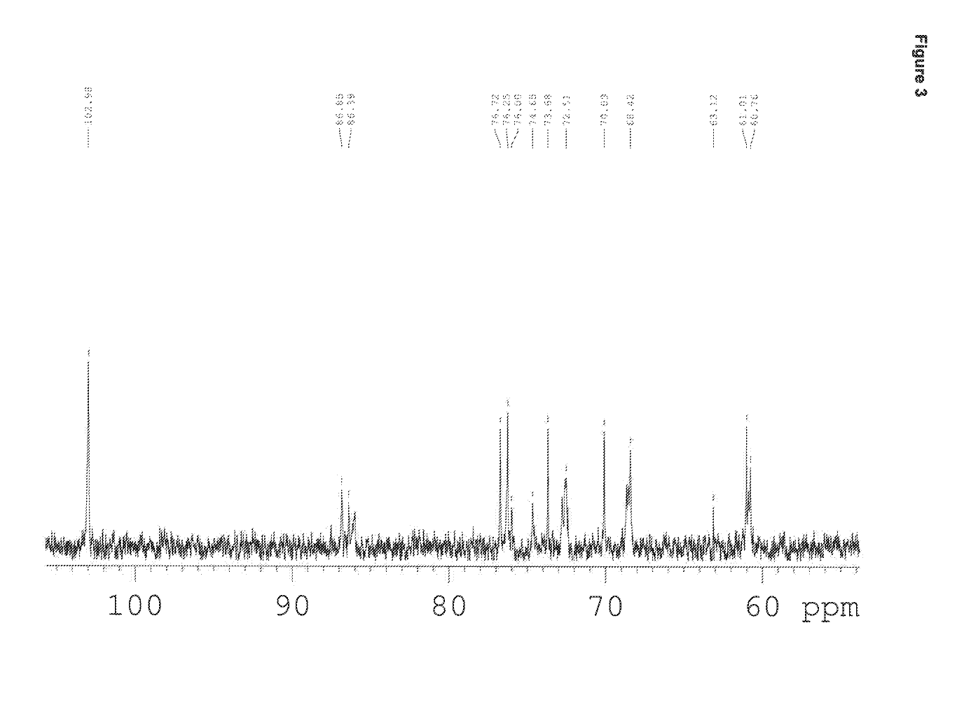

FIG. 3 13C-NMR of schizophyllan (50 mg of gel) in [D6]-DMSO measured at 50° C. (10.000 scans, 600 MHz), Assignment of the signals, δ (ppm): 60 and 61 (C-6), 68 (C6-C β(1-6)), 68 (C4-OH side glucose), 70 (C-2 backbone), 72 (C-2), 76 (C-5), 76.7 (C-3 side glucose), 86 (c-§ backbone), 103 (C-1).

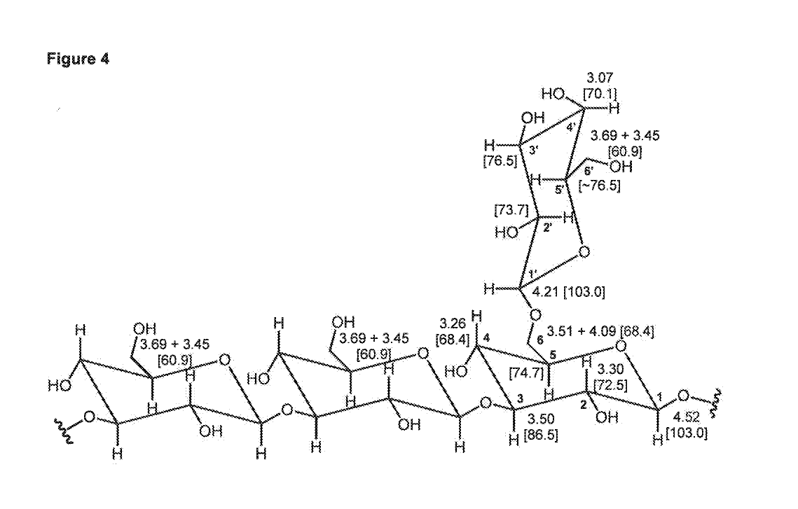

FIG. 4 Schematic picture of the repeating unit of schizophyllan.

The following Examples illustrate the present invention. Yet, the present invention must not be construed as being limited by the following Examples.

### EXAMPLES

### Example 1

### Cloning of the β-1,3-Glucan Synthase Expression Plasmid (pGS_{—}1) and Transformation into S. commune

In the genome of Schizophyllum commune, two genes encoding for β-1,3-glucan synthase were identified by using BLAST analysis (query genes: 1,3-β-glucan synthase sequence from Mycosphaerella graminicola, Saccharomyces cerevisiae, Cryptococcus neoformans, Schizosaccharomyces pombe); cf. Ullman, Biochem J (1997), 326: 929-942. In context of the present invention, it was proven that the overexpression of either of these β-1,3-glucan synthases in S. commune results in increased yields of schizophyllan production.

Two expression plasmids (pGS_{—}1)] and (pGS_{—}2) (having pBluescript II as backbone) were generated carrying selection marker cassette (amp^{R}, ura1), strong constitutive promoter (Tef1 promoter), the synthase gene sequence (genomic sequence) and terminator sequence (Tef1 terminator).

All polynucleotide sequences described herein originate from Schizopyllum commune. The polynucleotides represented by SEQ ID NOs 1 and 3 (genes β-1,3-glucan synthases I and II of Lu15531, respectively) were synthesized by Eurofins MWG GmbH/Germany (http://www.eurofinsdna.com/de) according to the original sequence data sourced from JGI data base (http://www.jgi.doe.gov/Scommune; gene position: scaffold 2, 1194740-1200474 and gene position: scaffold 6, 1391067-1396555). The sequences were delivered on pMK plasmids (pMK_GS_{—}1) and (pMK_GS_{—}2) (Eurofins plasmids containing kan^{R}, ColE1 origin and genomic sequence of respective β-1,3-glucan synthases). The polynucleotides were further used for the cloning of the complete expression plasmid. Plasmid (pMK_GS_{—}1) contained a polynucleotide represented by SEQ ID NO: 1 flanked by 5′ SpeI and 3′ SalI restriction sites. Plasmid (pMK_GS_{—}2) contained a polynucleotide represented by SEQ ID NO: 3 flanked by 5′ SpeI und 3′ EcoRV restriction sites, respectively.

The individual elements (SEQ ID NOs. 17, 18 and 33 (Tef1 promoter, Tef1 terminator and ura1) were isolated from the genomic DNA of Schizophyllum commune using PCR technology prepared by established microbiologic protocols (Sambrook, loc cit; Current Protocols in Molecular Biology, Update May 9, 2012, Print ISSN: 1934-3639, Online ISSN: 1934-3647).

All plasmid isolations were conducted according to manufacturer's instructions using HiSpeed Maxi Kit (Quiagen/Germany). For this purpose, Escherichia coli XL10 cells (Stratagene) containing the final expression plasmid or one of the interim plasmids were cultivated in Luria-Bertoni (LB) medium (Sigma-Aldrich) containing 50 mg/ml Ampicillin (Sigma-Aldrich).

For isolation of tef1 promoter sequence (SEQ ID NO: 17), 50 μl PCR reaction contained 1.25 U PfuUltra Hotstart Mastermix (Stratagene) and 1.25 U Taq PCR Mastermix (Quiagen), 22 μl H₂O, 22.1 pmol of forward primer TefP_forw (XbaI) (SEQ ID NO: 21) and 100 pmol of reverse primer TefP_rev (SpeI) (SEQ ID NO: 22), and 100 ng of template (genomic DNA of Schizophyllum commune). The reaction was carried out in Gene Amp® PCR System 9700 Thermal Cycler from PE Applied Biosystems. The following program was used for the amplification: initial heating step up to 95° C. for 4 minutes was followed by 30 cycles of 30 seconds denaturing at 95° C., 30 seconds of annealing step at 55° C., 1 minute elongation step at 72° C., followed by one cycle at 72° C. for 10 minutes.

For amplification of the synthetic β-1,3-glucan synthase gene (SEQ ID NO: 1), 50 μl PCR reaction contained 1.25 U PfuUltra Hotstart Mastermix (Stratagene) and 1.25 U Taq PCR Mastermix (Quiagen), 22 μl H₂O, 100 pmol of forward primer GS1_forw (SpeI) (SEQ ID NO: 27) and 22 pmol of reverse primer GS1_rev (SalI) (SEQ ID NO: 28), 100 ng template (pMK_GS_{—}1). The reaction was carried out in Gene Amp® PCR System 9700 Thermal Cycler from PE Applied Biosystems. The following program was used for the amplification: an initial heating step up to 95° C. for 4 minutes was followed by 30 cycles of 30 seconds denaturing at 95° C., 30 seconds of annealing step at 55° C., 8 minutes elongation step at 72° C., followed by one cycle at 72° C. for 10 minutes.

In the next PCR reaction step, fusion of the first two PCR products (tef1 promoter (SEQ ID NO: 17) with β-1,3-glucan synthase gene (SEQ ID NO: 1) was carried out. 50 μl PCR reaction contained 1.25 U of Pwo Hotstart Mastermix (Roche) and 1.25 U Taq PCR Mastermix (Quiagen), 22 μl of H₂O, 22.1 pmol of each primer: Fusion TefP_GS1_forw (XbaI) (SEQ ID NO: 29) and Fusion TefP_GS1_rev (SalI) (SEQ ID NO: 30) and 100 ng of both templates. The reaction was carried out in Gene Amp® PCR System 9700 Thermal Cycler from PE Applied Biosystems. The following program was used for the fusion of both sequences: an initial heating step up to 95° C. for 4 minutes was followed by 30 cycles of 30 seconds denaturing at 95° C., 30 seconds of annealing step at 55° C., 8 minutes elongation step at 72° C., followed by one cycle at 72° C. for 10 minutes.

The product of the fusion PCR was treated with SalI and XbaI restriction enzymes (Roche) according to manufacturer's instructions and the vector (pBluescript 2KSP, Stratagene Cloning Systems) was linearized using the same restriction enzymes and subsequently treated with alkaline phosphatase (Roche) according to manufacturer's instructions. Both, the digested PCR product and the linearized pBluescript 2KSP vector, were ligated using 14 DNA Ligase (New England Biolabs, Inc., Beverly, Mass./USA) and transformed into Escherichia coli XL10 cells (Stratagene) according to manufacturer's instructions.

For isolation of tef1 terminator sequence (SEQ ID NO: 18) following PCR reaction was carried out: 50 μl PCR reaction contained 1.25 U of Pwo Hotstart Mastermix (Roche) and 1.25 U Taq PCR Mastermix (Quiagen), 22 μl of H₂O, 24 pmol of forward primer TefT_forw (SalI) (SEQ ID NO: 23) and 21 pmol of reverse primer TefT_rev (SalI) (SEQ ID NO: 24), and 100 ng of template (genomic DNA of Schizophyllum commune). The reaction was carried out in Gene Amp® PCR System 9700 Thermal Cycler from PE Applied Biosystems. The following program was used: an initial heating step up to 95° C. for 4 minutes was followed by 30 cycles of 30 seconds denaturing at 95° C., 30 seconds of annealing step at 60° C., 1 minute elongation step at 72° C., followed by one cycle at 72° C. for 10 minutes. The PCR product was treated with SalI restriction enzyme (Roche) and ligated with the plasmid containing tef1 promoter and β-1,3-glucan synthase, which was before linearized with SalI restriction enzyme (Roche) and treated with alkaline phosphatase (Roche) according to manufacturer's instructions. After ligation, the DNA construct was transformed into Escherichia coli XL10 cells (Stratagene) according to manufacturer's instructions.

To enable screening of Schizophyllum commune strains after transformation with the β-1,3-glucan synthase expression, a plasmid selection marker (ura1; SEQ ID NO: 33) was introduced into the plasmid. For that purpose, ura1 gene was isolated from the genomic DNA of Schizophyllum commune. The PCR reaction contained 2.5 U of Pwo Hotstart Mastermix (Roche), 22 μl of H₂O, 21 pmol of forward primer Ura_forw (NotI) (SEQ ID NO: 19), 38 pmol of reverse primer Ura_rev (XbaI) (SEQ ID NO: 20) and 100 ng of the template (genomic DNA of Schizophyllum commune). The reaction was carried out in Gene Amp® PCR System 9700 Thermal Cycler from PE Applied Biosystems. The following program was used: an initial heating step up to 95° C. for 4 minutes was followed by 30 cycles of 30 seconds denaturing at 95° C., 30 seconds of annealing step at 60° C., 2 minutes elongation step at 72° C., followed by one cycle at 72° C. for 10 minutes. The PCR Product was digested with XbaI and NotI restriction enzymes (Roche) and ligated into the XbaI/NotI site of the β-1,3-glucan synthase expression plasmid (pGS_{—}1) using T4 DNA Ligase (New England Biolabs, Inc., Beverly, Mass./USA). The resulting plasmid encoding β-1,3-glucan synthase with tef1 promoter and terminator, and containing ura1 selection marker was transformed into Escherichia coli XL10 cells (Stratagene) according to manufacturer's instructions.

For the transformation of Schizophyllum commune with the β-1,3-glucan synthase expression plasmid (pGS_{—}1), plasmid preparation was carried out as follows. Escherichia coli XL10 cells containing the β-1,3-glucan synthase expression plasmid were cultivated in Luria-Bertoni (LB) medium (Sigma-Aldrich) containing 50 mg/ml Ampicillin (Sigma-Aldrich) and the plasmid isolation was conducted according to manufacturer's instructions using HiSpeed Maxi Kit (Quiagen).

Schizophyllum commune (Lu15527; obtained from strain collection of University of Jena (Germany), Prof. E. Kothe, Jena University internal strain name: 12-43) was transformed based on the method described by van Peer et al. (van Peer, loc cit) as basis. The method was modified according to the description below.

For preparation of S. commune protoplasts, fresh culture was inoculated on a plate containing complex medium (CYM). For incubation at 26° C. for 2-3 days, plates were sealed with parafilm.

For inoculation of liquid preculture (50 ml working volume), the biomass from the plate was macerated for 1 minute at 13500 rpm using T 25 digital ULTRA-TURRAX® (IKA), inoculated in shaking flask containing liquid CYM medium and incubated at 30° C., 220 rpm for further 3 days. Main culture was inoculated with 15 ml of the preculture in 200 ml CYM medium and incubated further 3 days at 30° C. at 220 rpm. After finishing the culture growth, the main culture was divided in four 50 ml samples and centrifuged (4000 rpm, 15 min). Obtained pellet was washed twice with 1 M MgSO₄ (50 ml) (Roth). After washing, four samples were united and dissolved 50 ml 1M MgSO₄.

To enable cell wall lysis, 100 mg Caylase (Cayla, Toulouse, France) were dissolved in 1 mL 1 M MgSO₄ and added to the pellet suspension. The sample was incubated over night at 30° C. under slight shaking (70 rpm). Subsequently distilled water was added to the sample (in 1:1 ratio), which was then incubated under slight shaking (70 rpm) for further 5 min. After this step, cells were incubated without shaking for 10 min and subsequently centrifuged (1100 rpm, 20 min, 4° C.). After the supernatant was filtrated using Miracloth-Membrane, one volume of cold 1 M sorbitol was added and the sample was allowed to equilibrate for 10 min. Subsequently, the sample was centrifuged (2000 rpm, 20 min, 2° C.). Pellet was washed by re-suspending carefully in 1 M sorbitol and centrifugation step was repeated. Finally the protoplasts were re-suspended in 1 M sorbitol and 50 mM CaCl₂ at a concentration of 10⁸ protoplasts per ml.

DNA used for transformation was a circular plasmid (pGS_{—}1) and the integration in the genome of S. commune was ectopic. To transform the protoplasts with the DNA, 100 μl protoplasts and 10 μl DNA (5-10 μg) were gently mixed and incubated for 60 min on ice. Subsequently, one volume of PEG 4000 (40%) was added and the sample was incubated for 5 to 10 min on ice. After adding 2.5 ml regeneration medium (complete medium containing 0.1 μg/ml Phleomycin and 0.5 M MgSO₄), the sample was incubated at 30° C., 70 rpm overnight.

After PEG mediated transformation, regenerated protoplasts were spread on petri dishes containing 40 ml solidified minimal medium: 2 g aspartic acid (Roth), 20 g glucose (Sigma), 0.5 g MgSO₄ (Roth), 0.5 g KH₂PO₄, 1 g K₂HPO₄ (both from Riedel-de Haën), 120 μg thiaminhydrochlorid (Roth) per liter, pH 6.3 containing 1% low melting agarose (Sigma). Selection plates were incubated 5 days at 30° C.

### Example 2

### Cloning of the β-1,3-Glucan Synthase Expression Plasmid [pGS_{—}2] and Transformation into S. commune

The expression plasmid for the second β-1,3-glucan synthase (SEQ ID NO: 3) (pGS_{—}2) was prepared analogously to the preparation of (pGS_{—}1) as described above in Example 1.

As a source of the promoter sequence tef1 (SEQ ID NO: 17); the same PCR product as in Example 1 was used.

Polynucleotide represented by SEQ ID NO: 3 was amplified from the (pMK_GS_{—}2) plasmid following PCR reaction: 50 μl PCR reaction contained 1.25 U PfuUltra Hotstart Mastermix (Stratagene) and 1.25 U Taq PCR Mastermix (Quiagen), 22 μl H₂O, 23 pmol of each primer: GS2_forw (SpeI)/SEQ ID NO: 31) and GS2_rev (EcoRV)(SEQ ID NO: 32), 100 ng of template (pMK_GS_{—}2). The reaction was carried out in Gene Amp® PCR System 9700 Thermal Cycler from PE Applied Biosystems. The following program was used for the amplification: an initial heating step up to 95° C. for 4 minutes was followed by 30 cycles of 30 seconds denaturing at 95° C., 30 seconds of annealing step at 53° C., 8 minutes elongation step at 72° C., followed by one cycle at 72° C. for 10 minutes.

For isolation of tef1 terminator sequence (SEQ ID NO: 18) and introduction of the EcoRV (5′) and ApaI (3′) sites, the following PCR reaction was carried out: 50 μl PCR reaction contained 1.25 U of Pwo Hotstart Mastermix (Roche) and 1.25 U Taq PCR Mastermix (Quiagen), 22 μl of H₂O, 37 pmol of forward primer TefT_forw (EcoRV) (SEQ ID NO: 25) and 25 pmol of reverse primer TefT_rev (ApaI)(SEQ ID NO: 26), and 100 ng of template (genomic DNA of Schizophyllum commune). The reaction was carried out in Gene Amp® PCR System 9700 Thermal Cycler from PE Applied Biosystems. The following program was used: an initial heating step up to 95° C. for 4 minutes was followed by 30 cycles of 30 seconds denaturing at 95° C., 30 seconds of annealing step at 58° C., 1 minute elongation step at 72° C., followed by one cycle at 72° C. for 10 minutes. The PCR product was treated with EcoRV and ApaI restriction enzyme (Roche) and ligated with the vector (pBluescript 2KSP, Stratagene Cloning Systems), which was before digested the same restriction enzymes. After ligation, the DNA construct was transformed into Escherichia coli XL10 cells (Stratagene), according to manufacturer's instructions.

Subsequently, tef1 promoter was cloned into the plasmid. For this purpose, the PCR product was digested with XbaI and SpeI (Roche) and ligated with the plasmid described above according to manufacturer's instructions, containing tef1 terminator which was linearized using XbaI and SpeI. The ligation was carried out as described in Example 1 herein. After ligation, the DNA construct was transformed into Escherichia coli XL10 cells (Stratagene) according to manufacturer's instructions.

Subsequently, ura1 was cloned into the plasmid. The same PCR product as in Example 1 was used. After digestion of the PCR product with NotI and XbaI, the fragment was cloned into the plasmid carrying the polynucleotide represented by SEQ ID NO: 7, tef1 promoter and terminator sequences. Before ligation, the plasmid was linearized by NotI and XbaI. Transformation was carried out as described above in Example 1.

Finally, β-1,3-glucan synthase (SEQ ID NO: 3) was ligated into the plasmid. For this purpose, the PCR product was treated with SpeI and EcoRV and ligated into the target expression plasmid as described above. Transformation was carried out as described above in Example 1.

Transformation of Schizophyllum commune with (pGS_{—}2) followed as described in Example 1.

### Example 3

### Verification of the Functionality of the Engineered S. commune Strains

Genetically modified S. commune strains generated as described above were tested in shaking flasks for increased schizophyllan production. To assure the reproducibility of the results, a three-step cultivation was applied, consisting of two pre-cultures and one main culture as further described herein below.

For the cultivation of the genetically modified Schizophyllum commune strains, two different media were used. For cultivation on solid media, CYM medium (25 g agar (Difco), 20 g glucose (Sigma), 2 g trypticase peptone (Roth), 2 g yeast extract (Difco), 0.5 g MgSO₄×7H₂O (Roth), 0.5 g KH₂PO₄ and 1 g K₂HPO₄ (both from Riedel-de Haën) per liter H₂O) was used. Strains were inoculated on agar plates containing CYM medium covered with cellophane (to avoid mycelium growth into the agar) and incubated for three to four days at 26° C.

For the liquid cultures, the following medium was used (hereinafter referred to as “Standard Medium”): 30 g glucose (Sigma), 3 g yeast extract (Difco), 1 g KH₂PO₄ (Riedel-de Haën), 0.5 g MgSO₄×7H₂O (Roth) per liter H₂O.

For both pre-cultures and for main culture, 250 ml shaking flasks filled with 30 ml Standard Medium were used. The cultivation was carried out at 27° C. and 225 rpm. Before each inoculation, the biomass was homogenized for 1 minute at 13500 rpm using T 25 digital ULTRA-TURRAX® (IKA).

The first pre-culture was inoculated with 50 mg of wet biomass. The cultures were incubated for 72 hours. After 72 hours, the second pre-culture was started. The concentration of the homogenized wet biomass from the first pre-culture used for inoculation was 250 mg. Cultivation time was 45 hours. After 45 hours, the main culture was inoculated with 500 mg of homogenized wet biomass from the second pre-culture and cultivated for another 45 hours.

After the cultivation was finished, standard analytical methods as described herein below were applied to define the biomass concentration, schizophyllan concentration, ethanol concentration and residual glucose in medium. 50 ml aliquots of the cultures were stabilized with 3 g/l Acticide BW20 (Thor).

Ethanol and glucose concentration was estimated using HPLC method. For this purpose 14 ml of the culture were centrifuged (30 min, 8500 rpm). The supernatant was sterile-filtrated and 1 ml of the filtrate was injected for the HPLC analysis (HPLC cation exchanger: Aminex HPX-87-H, BIO-RAD with 0.5 M H₂SO₄, Roth, as eluent and 0.5 ml/min flow rate at 30° C.).

Due to the fact that schizophyllan consists only of glucose molecules, the quantification of this polymer can be done using standard analytical methods for glucose. 10 ml of the culture, 20 ml H₂O and 90 μl Acticide BW20 were mixed. The sample was digested for 24 h at 40° C. with β-glucanase (0.3 ml) (Erbslöh). After the incubation, the sample was centrifuged (30 minutes at 3400 g) and the supernatant was analyzed for glucose and ethanol content using HPLC cation exchanger (Aminex HPX-87-H, BIO-RAD) with 0.5 M H₂SO₄ (Roth) as eluent and 0.5 ml/min flow rate at 30° C.

For the biomass determination, the remaining biomass in form of pellet (after β-glucanase digestion sample was centrifuged) was washed twice with 50 ml H₂O, filtrated using Whatman-Filter (with determination of filter's weight before filtration), washed twice with H₂O and dried in HB43S drying scale from Mettler Toledo. Drying of the filter was carried out for 5 to 10 minutes at 180° C. Subsequently, weight of the dry filter was determined.

The evaluation of the results obtained in shaking flasks showed clear effect of the overexpression of both β-1,3-glucan synthases on the schizophyllan production. Because of the fact that in the expression plasmid was ectopically integrated into genome and the integration locus has an explicit effect on the expression of the target gene, 40 clones carrying the plasmid (pGS_{—}1) and 40 clones carrying the plasmid (pGS_{—}2) were tested in shaking flask experiments. The increase of schizophyllan production in the genetically modified strains is shown in Table 1 in comparison to the non-modified Schizophyllum commune control strain. The results shown in the Table 1 refer to the best strain of each 40 strains tested. For classification of the strains, the amount of schizophyllan in the sample was decisive. 10 ml of the culture, 20 ml H₂O and 90 μl Acticide BW20 were mixed. The sample was digested for 24 h at 40° C. with 0.3 ml β-glucanase (Erbslöh). After the incubation, the sample was centrifuged (30 minutes at 3400 g) and the supernatant was analyzed for glucose and ethanol content using HPLC cation exchanger (Aminex HPX-87-H, BIO-RAD) with 0.5 M H₂SO₄ (Roth) as eluent and 0.5 ml/min flow rate at 30° C.

In addition to increased yields of schizophyllan production in the genetically modified S. commune strains, a clear decrease in the synthesis of the by-product ethanol was observed. This can be an indication that the excess rate of glucose by up-regulated β-1,3-glucan synthase activity is metabolized more directly in the schizophyllan pathway instead of partly being used for ethanol synthesis.

TABLE 1
Comparison of Schizophyllum commune control strain with
two genetically modified S. commune strains carrying glucan synthase
expression plasmid (pGS_1) or (pGS_2).

| | Schizophyllan | EtOH [% |
|---|---|---|
| Strain | [%] | [%] |
| S. commune control strain | 100 | 100 |
| S. commune (pGS_1) | 220 | 9 |
| S. commune (pGS_2) | 215 | 3.6 |

### Structure and Conformation Analysis of the Product

To assure that the polymer synthesized through genetically modified S. commune strains is schizophyllan, XRD and NMR methods were applied to confirm the structure of the molecule as follows.

Powder X-ray diffraction (XRD) allows rapid, non-destructive analysis of materials consisting of multiple components. Moreover, the sample preparation is straightforward. The data from the measurement is presented as a diffractogram in which the diffracted intensity (I) is shown as a function of scattering angle 2θ. The crystallinity of the given material can be determined by this measurement. In general, crystalline materials have reflection patterns of a series of sharp peaks whereas amorphous materials give a broad signals. Many polymers exhibit semicrystalline behaviour which can also be detected by XRD (Hammond, The basics of crystallography and diffraction, 3^{rd} Ed., Oxford University Press 2009).

Sample Preparation from Aqueous Solution

Aqueous solution containing schizophyllan was poured in ethanol to precipitate schizophyllan. The precipitation was filtered and dried either in a vacuum oven. The dried sample was measured by XRD.

### Sample Measurement and Results by XRD

Schizophyllan exhibits a triple helical structure. This was evident from the diffractogram of the precipitated and dried schizophyllan sample (FIG. 2). The triple helix could be seen as an intensive diffraction at 5° 2θ and the amorphous region of the material gives broad diffraction in the range of 20-25° 2θ (Hisamatsu, Carbohydr Res (1997), 298: 117).

### Sample Measurement and Results by NMR

The NMR spectra were recorded on a Varian VNMRS 600 MHz system equipped with a ¹³C-enhanced cryo probe (inverse configuration) at ambient temperatures or at 50° C. using standard pulse sequences for ¹H and ¹³C.

It is known that schizophyllan has a triple helical structure formed by three β(1-3)-D-glucan chains held together by hydrogen bonds in water. This structure is shielded in the magnetic field due to the rigid, ordered conformation. This means that in NMR spectrum, chemical shifts for schizophyllan are not obtained (Rinaudo, Carbohydr Polym (1982), 2: 135; Vlachou, Carbohydr Polym (2001), 46: 349) (2D NMR). In order to investigate the molecular structure of schizophyllan and not the macromolecular structure consisting of triple helices and further to record the successful NMR spectra with a good signal-to-noise ratio, the conformation of the triple helix has to be changed. It is also known that the triple helix of schizophyllan can be altered to form a random coil structure by addition of DMSO. When the DMSO concentration exceeds a certain threshold values (i.e. 87%), the conformation change takes place; therefore deuterated [D₆]-DMSO was used as a solvent for the measurements. This conformation matter is important to take into consideration when conducting NMR experiments for schizophyllan. Hence, the sample was measured in [D₆]-DMSO, the well-resolved spectra can be obtained (FIGS. 2 and 3).

### Summary

The chemical structures of the materials from S. commune (GS_{—}1) and S. commune (GS_{—}2) strain was identified to be the correct for that of schizophyllan. In addition, the materials exhibit the triple helix conformations.

### Sequences Referred to in the Present Application

TABLE 2
Assignment of SEQ ID NOs.

| SEQ | | |
|---|---|---|
| ID NO: | type of sequence | description |
| 1 | nucleotide sequence | Gene sequence* 1,3-β-D-glucan |
| | | synthase I of S. commune strain |
| | | Lu15531 |
| 2 | amino acid sequence | translation of SEQ ID NO: 5 |
| 3 | nucleotide sequence | Gene sequence* 1,3-β-D-glucan |
| | | synthase II of S. commune strain |
| | | Lu15531 |
| 4 | amino acid sequence | translation of SEQ ID NO: 7 |
| 5 | nucleotide sequence | cDNA 1,3-β-D-glucan |
| | | synthase I of S. commune strain |
| | | Lu15531 |
| 6 | amino acid sequence | polypeptide sequence 1,3-β-D-glucan |
| | | synthase I of S. commune strain |
| | | Lu15531 |
| 7 | nucleotide sequence | cDNA 1,3-β-D-glucan |
| | | synthase II of S. commune strain |
| | | Lu15531 |
| 8 | amino acid sequence | polypeptide sequence 1,3-β-D-glucan |
| | | synthase II of S. commune strain |
| | | Lu15531 |
| 9 | nucleotide sequence | Gene sequence* 1,3-β-D-glucan |
| | | synthase I of S. commune strain |
| | | Lu15634 |
| 10 | amino acid sequence | translation of SEQ ID NO: 13 |
| 11 | nucleotide sequence | Gene sequence* 1,3-β-D-glucan |
| | | synthase II of S. commune strain |
| | | Lu15634 |
| 12 | amino acid sequence | translation of SEQ ID NO: 15 |
| 13 | nucleotide sequence | cDNA 1,3-β-D-glucan |
| | | synthase I of S. commune strain |
| | | Lu15634 |
| 14 | amino acid sequence | polypeptide sequence 1,3-β-D-glucan |
| | | synthase I of S. commune strain |
| | | Lu15634 |
| 15 | nucleotide sequence | cDNA 1,3-β-D-glucan |
| | | synthase II of S. commune strain |
| | | Lu15634 |
| 16 | amino acid sequence | polypeptide sequence 1,3-β-D-glucan |
| | | synthase II of S. commune strain |
| | | Lu15634 |
| 17 | nucleotide sequence | tef1 promoter from S. commune |
| 18 | nucleotide sequence | tef1 terminator from S. commune |
| 19 | nucleotide sequence | Ura_forw (NotI) primer |
| 20 | nucleotide sequence | Ura_rev (XbaI) primer |
| 21 | nucleotide sequence | TefP_forw (XbaI) primer |
| 22 | nucleotide sequence | TefP_rev (SpeI) primer |
| 23 | nucleotide sequence | TefT_forw (SalI) primer |
| 24 | nucleotide sequence | TefT_rev (SalI) primer |
| 25 | nucleotide sequence | TefT_forw (EcoRV) primer |
| 26 | nucleotide sequence | TefT_rev (ApaI) primer |
| 27 | nucleotide sequence | GS1_forw (SpeI) primer |
| 28 | nucleotide sequence | GS1_rev (SalI) primer |
| 29 | nucleotide sequence | Fusion TefP_GS1_forw (XbaI) primer |
| 30 | nucleotide sequence | Fusion TefP_GS1_rev (SalI) primer |
| 31 | nucleotide sequence | GS2_forw (SpeI) primer |
| 32 | nucleotide sequence | GS2_rev (EcoRV) primer |
| 33 | nucleotide sequence | ura gene (S. commune) |
| 34 | amino acid sequence | Ura protein |

*Gene sequence includes introns and flanking regions. In the gene sequences below (for SEQ ID NOs. 1, 3, 9 and 11), predicted exons are shown in capital letters, introns are shown in lower case letters.

SEQ ID NO: 1
Gene sequence 1,3-β-D-glucan synthase I of S. commune
strain Lu15531
DNA
S. commune
CCCGTCCCTCAAGGCCGTTCTTTCGCTGGCGACCGACCCGGTGTTCGCGAGAACC
TGTTGTTTCTGACGATCATCAGCCCTTTCTTCTCGTCGCTCTTTAGCTCTCCCTAGA
CCGTCTTTTACTCTACTCTTCGACGCACGCCATGTCCGGCCCAGGATATGGCAGGA
ATCCATTCGACAATCCCCCGCCCAACAGAGGTCCCTATGGCCAGCAGCCAGGTTT
CCCGGGGCCCGGCCCTCGGCCTTACGACTCGGACGCGGACATGAGCCAGACCTA
TGGCAGCACAACCAGGCTCGCCGGCAGTGCCGGTTACAGCGACAGAAACGgtgcgc
acgtcgctaccgtacttcctcgatcgtcgattcacataccatgcagGCAGCTTCGACGGCGACCGCTCCTA
CGCGCCCTCAATTGACTCGCGCGCCAGCGTGCCCAGCATATCGCCCTTCGCAGAC
CCGGGTATCGGCTCTAATGAGCCGTATCCCGCTTGGTCGGTCGAACGCCAGATTC
CCATGTCCACGGAGGAGATTGAGGACATCTTCCTCGACCTCACCCAAAAGTTTGGC
TTCCAGCGCGACTCCATGCGGAATACGgtgcgtgaataagcagcccactcgaccgcgggaacagca
caattgacctgtcacccagTTCGACTTCATGATGCACCTCCTCGATTCCCGTGCCTCGCGCA
TGACGCCCAACCAAGCTCTGCTCACGCTTCACGCCGACTACATTGGTGGCCAGCA
TGCCAATTACCGGAAGTGGTATTTCGCCGCACAGCTCAACCTCGATGACGCGGTC
GGGCAAACCAATAACCCCGGTATCCAGCGCTTGAAGACCATCAAGGGCGCTACGA
AGACCAAGTCGCTCGACAGCGCACTCAACCGCTGGCGCAACGCGATGAACAACAT
GAGCCAGTACGATCGCCTCCGGCAAATTGCGCTCTACCTCCTCTGCTGGGGTGAA
GCAGGCAACATCCGTCTGGCGCCCGAGTGCTTGTGCTTCATCTTCAAGTGCGCGG
ACGACTACTACAGAAGTCCCGAGTGTCAGAACCGGATGGACCCCGTGCCGGAAGG
GCTGTACCTGCAGACGGTCATCAAGCCGCTCTATCGCTTCCTACGTGATCAGGCGT
ACGAAGTCGTTGATGGGAAGCAAGTGAAGCGCGAGAAGGACCACGACCAGATTAT
CGGTTATGACGACGTCAACCAGTTATTCTGGTATCCGGAAGGTTTGGCTAAGATCG
TCATGTCGGACAACgtgcgtatgatcttatcggttaaaattcgtccgctcacatctttccagACACGACTTGT
AGATGTACCTCCGGCGCAGCGGTTCATGAAGTTCGCCAAGATCGAGTGGAACCGC
GTCTTCTTCAAGACGTACTTTGAGAAGCGCTCTACTGCCCATCTCCTGGTCAACTTC
AACCGTATATGGATCCTCCACGTCTCGATGTACTTCTTCTACACGGCATTCAACTCT
CCACGAGTCTACGCGCCGCACGGCAAACTCGACCCCTCCCCTGAGATGACCTGGT
CCGCGACTGCCCTTGGAGGCGCTGTGTCCACCATGATCATGATCCTTGCCACTATC
GCGGAGTACACCTACATCCCCACGACATGGAACAATGCGTCGCACCTCACCACGC
GGCTCATTTTCCTCCTGGTCATCCTCGCGCTCACTGCTGGCCCAACATTCTATATC
GCCATGATAGACGGACGCACGGACATCGGCCAAGTACCACTCATCGTGGCCATAG
TGCAGTTCTTCATCTCCGTCGTCGCCACCCTCGCTTTCGCTACCATCCCTTCTGGT
CGCATGTTCGGCGACCGTGTGGCTGGCAAGTCAAGAAAGCACATGGCATCGCAGA
CGTTCACAGCGTCGTACCCGTCCATGAAGCGGTCATCTCGCGTAGCGAGTATCAT
GCTGTGGCTTTTGGTCTTTGGCTGCAAATACGTCGAGTCTTACTTCTTCTTGACGTC
CTCCTTCTCCAGCCCGATCGCGGTCATGGCGCGTACGAAGGTACAGGGCTGCAAC
GACCGTATCTTCGGCAGCCAGCTGTGCACGAATCAGGTCCCGTTCGCGCTGGCAA
TCATGTACGTGATGGACCTGGTACTGTTCTTCCTGGACACGTACCTGTGGTACATC
ATCTGGCTGGTGATCTTCTCGATGGTGCGCGCGTTCAAGCTTGGTATCTCGATCTG
GACGCCCTGGAGCGAGATCTTCACCCGCATGCCGAAGCGTATTTACGCAAAGCTG
CTGGCGACGGCCGAGATGGAGGTCAAGTATAAGCCCAAGgtatgctgaattcaatctggtcag
gtgaattcaccctcatattgtggtacagGTGCTCGTCTCACAAATCTGGAACGCGGTCATCATCTC
CATGTACCGGGAGCATCTCTTGTCCATCGAGCACGTCCAGCGCTTGCTTTACCACC
AGGTTGATGGTCCCGATGGCCGCCGCACCCTCAGGGCACCGCCGTTCTTCACCAG
CCAGCGAACTGCGAAGCCAGGCCTGTTCTTCCCTCCTGGTGGCGAGGCTGAGCGC
CGCATCTCGTTCTTTGCCTCATCGCTGACGACCGCGCTCCCGGAGCCTCTGCCGA
TCGACGCCATGCCCACCTTCACCGTGCTCGTTCCCCATTACTCCGAGAAGATTCTG
CTCAGTCTGCGCGAGATTATCCGCGAGGAGGACCAGAACACCCGCGTTACCTTAC
TGGAGTACCTCAAGCAGCTCCACCCTGTCGAATGGGACAATTTCGTCAAGGACACC
AAGATCTTGGCGGAAGAGTCGGGAGACGTCCAGGACGAGAAGCGCGCGCGCACG
GACGACTTGCCGTTCTATTGCATCGGGTTCAAGACCTCGTCACCAGAGTACACCCT
GCGTACGCGTATCTGGGCCTCACTGCGCGCACAGACGCTGTACCGCACGGTCTCC
GGTATGATGAACTACTCCAAGGCGATTAAGCTCCTCTATCGCGTCGAGAACCCGGA
TGTCGTTCATGCCTTCGGTGGGAACACGGAACGTCTTGAACGCGAGCTTGAGCGC
ATGTCTCGCCGCAAGTTCAAGTTCGTCATCTCGATGCAGCGGTACTCCAAGTTCAA
CAAGGAGGAGCAGGAGAACGCCGAGTTCCTTCTGCGCGCGTACCCGGATTTGCAG
ATCGCGTACCTCGATGAAGAGCCCGGTCCCAGCAAGAGCGACGAGGTTCGGTTGT
TTTCGACACTCATCGACGGACACTCCGAGGTGGACGAGAAGACGGGCCGCCGCAA
GCCCAAGTTCCGCATCGAGCTGCCCGGTAACCCCATCCTCGGTGACGGGAAGTCG
GATAACCAGAACCACGCCATCGTCTTCTACCGCGGCGAGTACATTCAGGTCATTGA
CGCTAACCAGGACAATTACCTGGAAGAGTGTCTCAAGATCCGTAATGTCCTGGGCG
AGTTTGAGGAATACTCCGTGTCGAGCCAGAGCCCGTACGCGCAGTGGGGCCACAA
GGAGTTCAACAAGTGCCCCGTCGCTATCCTGGGTTCCCGCGAGTACATCTTCTCG
GAGAACATCGGTATCCTCGGTGACATCGCTGCCGGCAAGGAACAGACGTTCGGTA
CCATTACGGCGCGTGCGCTTGCGTGGATCGGCGGCAAGCTGCATTACGGTCACCC
GGATTTCCTCAATGCGACGTTCATGACGACGCGTGGTGGCGTGTCAAAAGCGCAG
AAGGGCTTGCATCTTAACGAGGATATCTTCGCTGGTATGACCGCCGTGTCCCGCG
GAGGGCGCATCAAGCACATGGAGTACTACCAGTGCGGCAAAGGTCGTGATCTCGG
ATTCGGCACGATCTTGAACTTCCAGACCAAGATCGGTACTGGTATGGGCGAGCAG
CTGCTCTCGCGCGAGTACTACTATCTGGGCACGCAATTGCCTATCGACCGGTTCTT
GACGTTCTACTACGCGCACGCTGGTTTCCATGTCAACAACATCCTGGTCATCTACT
CCATCCAGGTCTTCATGGTCACCCgtaagtgcaggccctcatgaccgccgagcaagcagtctaacggat
gtgcagTGCTGTACCTGGGCACATTGAACAAGCAGCTGTTCATCTGCAAGGTCAACTC
CAATGGCCAGGTTCTTAGTGGACAAGCTGGGTGCTACAACCTCATCCCGGTCTTCG
AGTGGATTCGCCGGAGTATCATCTCCATCTTCTTGGTGTTCTTCATCGCCTTCTTGC
CGTTGTTCTTGCAAGgtatgttcacttctcatgtgccatttgtcaatcgctcactcgtacgacagAGCTTTGCG
AACGCGGAACAGGAAAGGCGTTGCTGCGTCTCGGGAAGCACTTCCTGTCACTGTC
GCCCATCTTCGAAGTGTTCTCCACCCAAATCTACTCGCAGGCGCTCTTGAACAACA
TGAGTTTCGGTGGTGCGCGCTACATCGCTACAGGACGCGGTTTCGCGACGAGTCG
GATACCCTTCAACATCCTCTACTCGCGTTTCGCGCCGCCGAGCATCTACATGGGCA
TGCGTAATCTGCTGCTCTTGCTGTACGCGACGATGGCCATTTGGATCCCACACCTG
ATCTACTTCTGGTTCTCCGTCCTCTCCCTCTGCATCGCGCCATTCATGTTCAATCCG
CATCAATTCTCGTACGCTGACTTCATCATCGACTACCGGGAGTTCTTGCGCTGGAT
GTCGCGCGGTAACTCGCGGACGAAGGCGAGTAGCTGGTACGGATATTGCCGTCTG
TCGCGTACCGCGATTACTGGGTACAAGAAGAAGAAACTGGGACACCCGTCGGAGA
AGCTGTCGGGCGATGTGCCGCGTGCGCCGTGGAGGAACGTCATCTTCTCGGAGAT
CCTTTGGCCCATCGGCGCGTGCATCATCTTCATCGTCGCGTACATGTTCGTCAAAT
CGTTCCCTGACGAGCAGGGCAACGCGCCGCCGAGCCCGCTGGTCCGCATTCTGC
TCATCGCGGTTGGCCCTACTGTGTGGAACGCGGCGGTGCTCATCACGCTGTTCTT
CCTGTCGCTCTTCCTGGGCCCGATGATGGATGGCTGGGTCAAGTTCGGCTCAGTC
ATGGCGGCACTTGCGCATGGTCTAGCGCTCATAGGCATGCTCACGTTCTTCGAGTT
CTTCgtacgtccttcgcgttgttgtggtcgagtgctttgctaacaccgccttcagTGGTTCCTCGAGCTCTGGG
ATGCCTCGCACGCCGTGCTCGGCGTCATCGCCATTATTGCCGTTCAGCGCGGGAT
CCAGAAGATCCTCATTGCCGTCTTCCTGACGCGTGAGTACAAGCACGACGAGACG
AACCGCGCGTGGTGGACAGGTAAATGGTATGGACGCGGGCTGGGTACCTCGGCC
ATGTCCCAGCCGGCGCGCGAGTTCATCGTGAAGATCGTGGAGATGTCGCTGTGGA
CGTCGGACTTCCTGCTTGCGCACCTGTTGCTCATCATCTTGACGGTGCCGCTACTG
CTGCCGTTCTTCAACTCGATCCATTCGACGATGCTTTgtgagtgatttgtagtcgttggtcacggat
gattgctgactcgcgtgcagTCTGGTTGCGCCCTTCGAAGCAGATTAGGCAACCTCTGTTCT
CCACTAAGCAGAAGCGGCAACGGCGATGGATTgtaagttcctttgattgctctggctaccgaccttcgc
tcacctgtctcagGTCATGAAGTATACCGTGGTATATCTCGTGGTGGTGGCTTTCCTCGTT
GCGCTCATCGCTCTGCgtacgttttctgtcgcgctcaccctctattttcactaacgtttcctccagCCGCGCTC
TTCCGCGAGAGCATCCACTTCAACTGCGAGATCTGCCAGAGTATATAGTCATATAA
CGACGTCTATCGTATCGCCGGACGAGAGCCCCGTCGCCTACACACTGACATGGAA
TTGCTGTGTATACAATCGATCTTCTGACCGCGTCGGGGGCGTTGCCGTCTTTCTAC
TATCAACTTGCTTGTGTATCAACATTTCTTCTCTCCAAGCCTACATTGACATAGAGTA
ATAGCCCATGTTCATACAACAATCGCATAGCATTGCATATACCAT
SEQ ID NO: 2
Translation of SEQ ID NO: 5
amino acid
S. commune
MSGPGYGRNPFDNPPPNRGPYGQQPGFPGPGPRPYDSDADMSQTYGSTTRLAGSA
GYSDRNGSFDGDRSYAPSIDSRASVPSISPFADPGIGSNEPYPAWSVERQIPMSTEEIE
DIFLDLTQKFGFQRDSMRNTFDFMMHLLDSRASRMTPNQALLTLHADYIGGQHANYRK
WYFAAQLNLDDAVGQTNNPGIQRLKTIKGATKTKSLDSALNRWRNAMNNMSQYDRLR
QIALYLLCWGEAGNIRLAPECLCFIFKCADDYYRSPECQNRMDPVPEGLYLQTVIKPLY
RFLRDQAYEVVDGKQVKREKDHDQIIGYDDVNQLFWYPEGLAKIVMSDNTRLVDVPPA
QRFMKFAKIEWNRVFFKTYFEKRSTAHLLVNFNRIWILHVSMYFFYTAFNSPRVYAPHG
KLDPSPEMTWSATALGGAVSTMIMILATIAEYTYIPTTVVNNASHLTTRLIFLLVILALTAGP
TFYIAMIDGRTDIGQVPLIVAIVQFFISVVATLAFATIPSGRMFGDRVAGKSRKHMASQTF
TASYPSMKRSSRVASIMLWLLVFGCKYVESYFFLTSSFSSPIAVMARTKVQGCNDRIFG
SQLCTNQVPFALAIMYVMDLVLFFLDTYLWYIIWLVIFSMVRAFKLGISIWTPWSEIFTRM
PKRIYAKLLATAEMEVKYKPKVLVSQIWNAVIISMYREHLLSIEHVQRLLYHQVDGPDGR
RTLRAPPFFTSQRTAKPGLFFPPGGEAERRISFFASSLTTALPEPLPIDAMPTFTVLVPH
YSEKILLSLREIIREEDQNTRVTLLEYLKQLHPVEWDNFVKDTKILAEESGDVQDEKRAR
TDDLPFYCIGFKTSSPEYTLRTRIWASLRAQTLYRIVSGMMNYSKAIKLLYRVENPDVV
HAFGGNTERLERELERMSRRKFKFVISMQRYSKFNKEEQENAEFLLRAYPDLQIAYLDE
EPGPSKSDEVRLFSTLIDGHSEVDEKTGRRKPKFRIELPGNPILGDGKSDNQNHAIVFY
RGEYIQVIDANQDNYLEECLKIRNVLGEFEEYSVSSQSPYAQWGHKEFNKCPVAILGSR
EYIFSENIGILGDIAAGKEQTFGTITARALAWIGGKLHYGHPDFLNATFMTTRGGVSKAQ
KGLHLNEDIFAGMTAVSRGGRIKHMEYYQCGKGRDLGFGTILNFQTKIGTGMGEQLLS
REYYYLGTQLPIDRFLTFYYAHAGFHVNNILVIYSIQVFMVILLYLGTLNKQLFICKVNSN
GQVLSGQAGCYNLIPVFEWIRRSIISIFLVFFIAFLPLFLQELCERGTGKALLRLGKHFLSL
SPIFEVFSTQIYSQALLNNMSFGGARYIATGRGFATSRIPFNILYSRFAPPSIYMGMRNLL
LLLYATMAIWIPHLIYFWFSVLSLCIAPFMFNPHQFSYADFIIDYREFLRWMSRGNSRTK
ASSWYGYCRLSRTAITGYKKKKLGHPSEKLSGDVPRAPWRNVIFSEILWPIGACIIFIVAY
MFVKSFPDEQGNAPPSPLVRILLIAVGPTVWNAAVLITLFFLSLFLGPMMDGWVKFGSV
MAALAHGLALIGMLTFFEFFWFLELWDASHAVLGVIAIIAVQRGIQKILIAVFLTREYKHDE
TNRAWWTGKWYGRGLGTSAMSQPAREFIVKIVEMSLWTSDFLLAHLLLIILTVPLLLPFF
NSIHSTMLFWLRPSKQIRQLFSTKQKRQRRWIVMKYTVVYLVVVAFLVALIALPALFRE
SIHFNCEICQSI
SEQ ID NO: 3
Gene sequence 1,3-β-D-glucan synthase II of
S. commune strain Lu15531
DNA
S. commune
CTGTCCAAAGAAGAGATCGAGGACATCTTCCTCGATCTGACGCAGAAGTTTGGCTT
TCAGCGGGATTCCATGCGGAACATGgtacgtggcgtatgcccatgtgcggcgttctgaggcctaaacgttt
tccgccagTTCGACTTCACCATGCAGCTGCTTGACAGCCGAGCGTCTCGTATGACCCC
CAACCAGGCGCTCCTCACCCTCCACGCCGACTACATTGGTGGCCAGCATGCGAAC
TACCGGAAGTGGTACTTCGCGGCGCAGCTCGACCTTGACGACGCCGTGGGACAAA
CTCAGAATCCGGGTCTCAACCGCCTCAAGTCCACTCGCGGATCGGGCAAGCGACC
ACGCCATGAAAAGTCGCTGAACACGGCATTGGAGCGCTGGCGGCAAGCCATGAAC
AACATGTCGCAGTATGACCGCTTACGCCAGATCGCGCTCTACCTGCTCTGCTGGG
GCGAAGCGGCGCAAGTGCGATTCATGCCCGAGTGCTTGTGCTTCATCTTCAAGTG
CGCCGACGACTATTATCGTTCGCCGGAGTGCCAGAACAGGATGGAGCCGGTACCG
GAGGGTCTCTACCTGAGGACGGTCGTAAAGCCGCTCTACAGATTTGTCCGGGATC
AAGGCTATGAGGTGGTGGAGGGAAAATTCGTACGGCGGGAACGGGATCACGACCA
AATCATTGGTTACGATGACGTGAATCAGCTGTTCTGGTACCCGGAGGGCATTGCCC
GTATCGTCCTGTCGGACAAGgtaagcacctctgtgcatcttctgtgacatacagggctaattgtcgagcagA
GTCGTCTGGTCGACCTCCCTCCAGCACAGCGCTTCATGAAGTTCGACCGTATCGA
GTGGAATCGCGTCTTCTTCAAGACGTTCTACGAGACTCGATCCTTTACGCATCTTTT
GGTCGACTTCAACCGTATCTGGGTCGTGCACATCGCTCTCTACTTCTTCTACACCG
CATACAACTCCCCCACGATCTACGCCATCAACGGCAACACTCCGACGTCTCTGGCT
TGGAGCGCGACTGCGCTCGGCGGTGCGGTAGCGACAGGTATCATGATCCTCGCC
ACGATCGCCGAGTTCTCGCACATCCCCACGACATGGAACAACACCTCGCATCTGAC
TCGCCGCCTCGCCTTCCTCCTCGTCACGCTCGGCCTCACATGTGGTCCGACGTTC
TACGTCGCGATTGCAGAGAGCAACGGGAGCGGCGGCTCTTTGGCCTTGATTCTCG
GCATCGTCCAGTTCTTCATCTCCGTCGTAGCGACTGCGCTCTTCACTATCATGCCTT
CTGGTCGTATGTTCGGCGACCGCGTCGCAGGCAAGAGTCGCAAGTATCTCGCCAG
CCAGACGTTCACGGCCAGCTACCCGTCGTTGCCCAAGCACCAGCGGTTCGCATCA
CTCCTGATGTGGTTCCTCATCTTCGGGTGCAAGTTGACGGAGAGTTACTTCTTCCT
GACGTTGTCCTTCCGCGACCCTATTCGCGTCATGGTCGGCATGAAGATCCAGAACT
GCGAGGACAAGATTTTCGGCAGCGGCCTTTGCAGGAATCACGCAGCATTCACCCT
CACGATCATGTACATCATGGACCTCGTCTTGTTCTTCCTCGACACCTTCCTTTGGTA
TGTCATCTGGAACTCGGTTTTCAGTATCGCACGCTCTTTCGTACTCGGCCTTTCGAT
CTGGACACCATGGAGGGACATCTTCCAGCGTCTGCCGAAGCGTATCTACGCGAAG
CTTCTAGCGACCGGCGACATGGAGGTCAAGTACAAGCCCAAGgtgtgtgaatagctcgctgt
aaggttcttgattctgactcattcgcagGTCTTGGTTTCGCAAATCTGGAACGCCATCATCATCTC
CATGTACCGCGAGCACTTGCTCTCTATCGAGCACGTTCAAAAGCTCCTGTACCATC
AAGTGGACACTGGCGAAGCCGGCAAGCGGAGTCTTCGCGCGCCTCCGTTCTTCGT
CGCGCAGGGCAGCAGCGGTGGCTCGGGCGAGTTCTTCCCGCCTGGTAGCGAGGC
TGAGCGTCGTATCTCTTTCTTCGCGCAGTCTCTATCTACGGAGATTCCTCAGCCCAT
CCCGGTTGACGCCATGCCGACGTTCACAGTGCTTACGCCTCACTACAGCGAGAAG
gtgcgctttttcctgggcgcattcaacattagctgactgtcgtgcacagATCCTTCTTTCGCTCCGTGAGATT
ATCCGCGAGGAGGACCAGAACACCCGCGTGACATTGCTTGAGTATCTCAAGCAGC
TTCACCCGGTCGAGTGGGAGAACTTCGTCAAGGACACCAAGATTTTGGCCGAGGA
GTCCGCTATGTTCAACGGTCCAAGTCCTTTCGGCAACGATGAGAAGGGTCAGTCCA
AGATGGACGATCTTCCTTTCTACTGCATCGGTTTCAAGAGCGCCGCGCCCGAGTAC
ACCCTCCGCACCCGTATCTGGGCGTCCTTGCGCGCGCAGACCCTCTACCGCACGG
TCTCCGGCATGATGAACTATGCGAAGGCGATTAAGCTGCTCTACCGCGTCGAGAAC
CCCGAGGTCGTGCAGCAGTTCGGCGGTAACACGGACAAGCTCGAGCGCGAGTTG
GAGCGGATGGCCCGGCGGAAGTTCAAGTTCCTGGTGTCCATGCAGCGCTACTCGA
AGTTCAACAAGGAGGAGCACGAGAACGCCGAGTTCTTGCTCCGCGCGTACCCGGA
CCTGCAGATCGCGTACCTGGAGGAAGAGCCTCCTCGCAAGGAGGGTGGCGATCC
ACGCATCTTCTCTGCCCTCGTCGACGGCCACAGCGACATCATCCCGGAGACCGGC
AAGCGGCGCCCCAAGTTCCGCATCGAGCTGCCCGGCAACCCCATTCTCGGTGACG
GCAAGTCGGACAACCAGAACCACGCCATCGTCTTCTACCGCGGCGAGTACCTCCA
GCTTATCGACGCCAACCAGGACAACTACCTCGAGGAGTGCTTGAAGATCCGTAAC
GTACTCGCCGAGTTCGAGGAGTACGACGTCTCTAGCCAGAGTCCGTACGCGCAGT
GGAGTGTCAAGGAGTTCAAGCGCTCCCCGGTCGCCATCGTCGGTGCACGCGAGTA
TATCTTCTCGGAGCACATCGGTATTCTCGGTGATTTGGCGGCTGGCAAGGAACAGA
CGTTCGGTACGCTCACGGCACGCAACAACGCCTTCCTTGGCGGCAAGCTGCACTA
CGGTCACCCGGATTTCCTCAACGCCCTCTACATGAACACGCGCGGTGGTGTCTCC
AAGGCGCAGAAGGGTCTCCATCTCAACGAGGATATTTACGCCGGTATGAACGCGG
TCGGTCGCGGTGGACGCATCAAGCATAGCGAATACTACCAGTGCGGCAAGGGTCG
TGACCTCGGTTTTGGCACCATCTTGAACTTCCAGACCAAGATCGGTACGGGTATGG
GCGAGCAGATCCTCTCGCGCGAGTACTACTACCTCGGAACCCAATTGCCCATCGAT
CGCTTCCTCACGTTCTACTACGCGCACCCAGGTTTCCAGATCAACAACATGCTGGT
TATCCTATCCGTGCAGGTCTTCATCGTTACCAgtacgttgattgcatatcgttagcctgacagcgtctga
cgaattcccagTGGTCTTCCTCGGTACCTTGAAGTCTTCGGTCACGATCTGCAAGTACAC
GTCCAGCGGTCAGTACATCGGTGGTCAATCCGGTTGCTACAACCTCGTCCCGGTC
TTCCAGTGGATCGAGCGCTGCATCATCAGCATCTTCTTGGTGTTCATGATCGCTTTC
ATGCCGCTCTTCCTGCAAGgtaagagctcgtcaacctgctcaagggccttgcgctgatcatcattcagAAC
TCGTCGAGCGCGGTACCTGGAGTGCCATCTGGCGTCTGCTCAAGCAGTTTATGTC
GCTGTCGCCTGTCTTCGAGGTGTTCTCCACCCAGATTCAGACACACTCCGTGTTGA
GCAACTTGACGTTCGGTGGTGCGCGTTACATCGCTACCGGTCGTGGGTTCGCCAC
CAGTCGTATCAGCTTCAGCATCTTGTTCTCGCGTTTCGCAGGCCCGAGTATCTACC
TCGGCATGCGCACGCTCATTATGCTGCTCTACGTGACGTTGACGATCTGGACGCCA
TGGGTCATTTACTTCTGGGTTTCCATTCTCTCGCTCTGCATCGCGCCGTTCTTGTTC
AATCCGCATCAATTCGTCTTCTCGGATTTCCTCATCGACTACAGgtacgtcggacgagcgct
gttccgcgacgtaagctgaccggttatacagGGAATACCTCCGGTGGATGTCGCGTGGTAACTCG
CGCTCGCACAACAACTCCTGGATTGGGTACTGCCGGTTGTCCCGCACGATGATCA
CTGGGTACAAGAAGAAGAAGCTGGGCCACCCGTCGGAGAAGCTTTCCGGCGACGT
TCCTCGTGCAGGCTGGCGCGCCGTCTTATTCTCGGAGATCATCTTCCCGGCATGC
ATGGCCATCCTCTTCATCATCGCGTACATGTTCGTCAAGTCGTTCCCTCTCGACGG
CAAGCAGCCTCCCTCCGGCCTCGTTCGCATCGCCGTCGTGTCTATCGGCCCCATC
GTGTGGAACGCCGCCATCCTGTTGACGCTCTTCCTTGTGTCGTTGTTCCTCGGCCC
CATGCTCGACCCGGTCTTCCCCCTCTTCGGTTCCGTTATGGCCTTCATCGCGCATT
TCCTCGGCACAATCGGAATGATTGGGTTCTTCGAGTTCCTGgtatgtgcccatacctttcattcgt
cttcaactatctaacagattcatagTGGTTCCTCGAGTCCTGGGAGGCGTCGCATGCCGTGCTG
GGTCTCATCGCCGTCATCTCCATCCAGCGCGCCATTCACAAAATTCTTATCGCCGT
TTTCCTCAGTCGCGAGTTCAAGCACGACGAGACGAACAGGGCTTGGTGGACTGGT
CGCTGGTATGGCCGTGGCCTCGGCACGCACGCCATGTCGCAGCCGGCGCGTGAG
TTCGTCGTCAAGATCATCGAGTTGTCGCTCTGGAGCTCGGATCTCATACTCGGCCA
CATCCTGCTGTTCATGCTTACTCCGGCTGTCCTCATCCCGTACTTCGACCGTCTGC
ACGCCATGATGCTCTgtacgtcgtgtctcattgtttgtgttggtcatactcttaccctctcttagTCTGGCTGCG
CCCCTCAAAGCAAATCCGCGCGCCTCTGTACTCAATCAAGCAGAAGAGGCAAAGA
CGCTGGATTgtcagtgttcagtgccttattctatcagctcttactgacgtcttcatagATCATGAAGTACGGTA
CTGTATACGTTACCGTCATCGCGATCTTCGTCGCGCTCATCGCGCTTCgtgagtacccttg
ctatctttcgtacctgagcgtcgctgacccctttcccagCCCTCGTCTTCCGACACACTCTAAAGGTCGA
GTGCTCCCTTTGCGACAGCTTGTAATATCGGACTCGTATATATCTAGACTTCTCCGC
ACCATGTGTAGCTGACGCTTGGGTATACTTCGCGGTGCCGAGCTAATTGTCGACG
GACATTCTCCATCGTTGAGTGCAGCGACATCGGGTGGTTTACGACACGGACACTTT
TCATTGTACCCTCTACGAATGCAAGAACTCTCTTACGACCAGTACCTATGTGCTAAG
CCGTCGCCTGTTCAGGATCATACATACATACGTTTCTAGATACCTTACAGTTAGGCC
TATTCAGGGAGAGTCTGCATAAAA
SEQ ID NO: 4
Translation of SEQ ID NO: 7
amino acid
S. commune
MRNMFDFTMQLLDSRASRMTPNQALLTLHADYIGGQHANYRKWYFAAQLDLDDAVGQ
TQNPGLNRLKSTRGSGKRPRHEKSLNTALERWRQAMNNMSQYDRLRQIALYLLCWG
EAAQVRFMPECLCFIFKCADDYYRSPECQNRMEPVPEGLYLRTVVKPLYRFVRDQGY
EVVEGKFVRRERDHDQIIGYDDVNQLFWYPEGIARIVLSDKSRLVDLPPAQRFMKFDRI
EWNRVFFKTFYETRSFTHLLVDFNRIINVVHIALYFFYTAYNSPTIYAINGNTPTSLAWSA
TALGGAVATGIMILATIAEFSHIPTTINNNTSHLTRRLAFLLVTLGLTCGPTFYVAIAESNG
SGGSLALILGIVQFFISWATALFTIMPSGRMFGDRVAGKSRKYLASQTFTASYPSLPKH
QRFASLLMWFLIFGCKLTESYFFLTLSFRDPIRVMVGMKIQNCEDKIFGSGLCRNHAAFT
LTIMYIMDLVLFFLDTFLVVYVIWNSVFSIARSFVLGLSIVVTPWRDIFQRLPKRIYAKLLATG
DMEVKYKPKVLVSQIWNAIIISMYREHLLSIEHVQKLLYHQVDTGEAGKRSLRAPPFFVA
QGSSGGSGEFFPPGSEAERRISFFAQSLSTEIPQPIPVDAMPTFTVLTPHYSEKILLSLR
EIIREEDQNTRVTLLEYLKQLHPVEWENFVKDTKILAEESAMFNGPSPFGNDEKGQSKM
DDLPFYCIGFKSAAPEYTLRTRIWASLRAQTLYRTVSGMMNYAKINKLLYRVENPEVVQ
QFGGNTDKLERELERMARRKFKFLVSMQRYSKFNKEEHENAEFLLRAYPDLQIAYLEE
EPPRKEGGDPRIFSALVDGFISDIIPETGKRRPKFRIELPGNPILGDGKSDNQNHAIVFYR
GEYLQLIDANQDNYLEECLKIRNVLAEFEEYDVSSQSPYAQWSVKEFKRSPVAIVGARE
YIFSEHIGILGDLAAGKEQTFGTLTARNNAFLGGKLHYGHPDFLNALYMNTRGGVSKAQ
KGLHLNEDIYAGMNAVGRGGRIKHSEYYQCGKGRDLGFGTILNFQTKIGTGMGEQILS
REYYYLGTQLPIDRFLTFYYAHPGFQINNMLVILSVQVFIVTIMVFLGTLKSSVTICKYTSS
GQVIGGQSGCYNLVPVKIWIERCIISIFLVFMIAFMPLFLQELVERGTVVSAIWRLLKQFM
SLSPVFEVFSTQIQTHSVLSNLTFGGARYIATGRGFATSRISFSILFSRFAGPSIYLGMRT
LIMLLYVTLTIVVTPWVIYFWVSILSLCIAPFLFNPHQFVFSDFLIDYREYLRWMSRGNSRS
HNNSWIGYCRLSRTMITGYKKKKLGHPSEKLSGDVPRAGWRAVLFSEIIFPACMAILFIIA
YMFVKSFPLDGKQPPSGLVRIAVVSIGPINNVNAAILLTLFLVSLFLGPMLDPVFPLFGSV
MAFIAHFLGTIGMIGFFEFLWFLESWEASHAVLGLIAVISIQRAINKILIAVFLSREFKHDET
NRAwWTGRVVYGRGLGTHAMSQPAREFVVKIIELSLWSSDLILGHILLFMLTPAVLIPYF
DRLHAMMLFWLRPSKQIRAPLYSIKQKRQRRWIIMKYGTVYVTVIAIFVALIALPLVFRFIT
LKVECSLCDSL
SEQ ID NO: 5
cDNA 1,3-β-D-glucan synthase i of S. commune strain Lu15531
DNA
S. commune
ATGTCCGGCCCAGGATATGGCAGGAATCCATTCGACAATCCCCCGCCCAACAGAG
GTCCCTATGGCCAGCAGCCAGGTTTCCCGGGGCCCGGCCCTCGGCCTTACGACTC
GGACGCGGACATGAGCCAGACCTATGGCAGCACAACCAGGCTCGCCGGCAGTGC
CGGTTACAGCGACAGAAACGGCAGCTTCGACGGCGACCGCTCCTACGCGCCCTCA
ATTGACTCGCGCGCCAGCGTGCCCAGCATATCGCCCTTCGCAGACCCGGGTATCG
GCTCTAATGAGCCGTATCCCGCTTGGTCGGTCGAACGCCAGATTCCCATGTCCAC
GGAGGAGATTGAGGACATCTTCCTCGACCTCACCCAAAAGTTTGGCTTCCAGCGC
GACTCCATGCGGAATACGTTCGACTTCATGATGCACCTCCTCGATTCCCGTGCCTC
GCGCATGACGCCCAACCAAGCTCTGCTCACGCTTCACGCCGACTACATTGGTGGC
CAGCATGCCAATTACCGGAAGTGGTATTTCGCCGCACAGCTCAACCTCGATGACGC
GGTCGGGCAAACCAATAACCCCGGTATCCAGCGCTTGAAGACCATCAAGGGCGCT
ACGAAGACCAAGTCGCTCGACAGCGCACTCAACCGCTGGCGCAACGCGATGAACA
ACATGAGCCAGTACGATCGCCTCCGGCAAATTGCGCTCTACCTCCTCTGCTGGGG
TGAAGCAGGCAACATCCGTCTGGCGCCCGAGTGCTTGTGCTTCATCTTCAAGTGC
GCGGACGACTACTACAGAAGTCCCGAGTGTCAGAACCGGATGGACCCCGTGCCG
GAAGGGCTGTACCTGCAGACGGTCATCAAGCCGCTCTATCGCTTCCTACGTGATCA
GGCGTACGAAGTCGTTGATGGGAAGCAAGTGAAGCGCGAGAAGGACCACGACCA
GATTATCGGTTATGACGACGTCAACCAGTTATTCTGGTATCCGGAAGGTTTGGCTA
AGATCGTCATGTCGGACAACACACGACTTGTAGATGTACCTCCGGCGCAGCGGTT
CATGAAGTTCGCCAAGATCGAGTGGAACCGCGTCTTCTTCAAGACGTACTTTGAGA
AGCGCTCTACTGCCCATCTCCTGGTCAACTTCAACCGTATATGGATCCTCCACGTC
TCGATGTACTTCTTCTACACGGCATTCAACTCTCCACGAGTCTACGCGCCGCACGG
CAAACTCGACCCCTCCCCTGAGATGACCTGGTCCGCGACTGCCCTTGGAGGCGCT
GTGTCCACCATGATCATGATCCTTGCCACTATCGCGGAGTACACCTACATCCCCAC
GACATGGAACAATGCGTCGCACCTCACCACGCGGCTCATTTTCCTCCTGGTCATCC
TCGCGCTCACTGCTGGCCCAACATTCTATATCGCCATGATAGACGGACGCACGGA
CATCGGCCAAGTACCACTCATCGTGGCCATAGTGCAGTTCTTCATCTCCGTCGTCG
CCACCCTCGCTTTCGCTACCATCCCTTCTGGTCGCATGTTCGGCGACCGTGTGGCT
GGCAAGTCAAGAAAGCACATGGCATCGCAGACGTTCACAGCGTCGTACCCGTCCA
TGAAGCGGTCATCTCGCGTAGCGAGTATCATGCTGTGGCTTTTGGTCTTTGGCTGC
AAATACGTCGAGTCTTACTTCTTCTTGACGTCCTCCTTCTCCAGCCCGATCGCGGT
CATGGCGCGTACGAAGGTACAGGGCTGCAACGACCGTATCTTCGGCAGCCAGCTG
TGCACGAATCAGGTCCCGTTCGCGCTGGCAATCATGTACGTGATGGACCTGGTACT
GTTCTTCCTGGACACGTACCTGTGGTACATCATCTGGCTGGTGATCTTCTCGATGG
TGCGCGCGTTCAAGCTTGGTATCTCGATCTGGACGCCCTGGAGCGAGATCTTCAC
CCGCATGCCGAAGCGTATTTACGCAAAGCTGCTGGCGACGGCCGAGATGGAGGTC
AAGTATAAGCCCAAGGTGCTCGTCTCACAAATCTGGAACGCGGTCATCATCTCCAT
GTACCGGGAGCATCTCTTGTCCATCGAGCACGTCCAGCGCTTGCTTTACCACCAG
GTTGATGGTCCCGATGGCCGCCGCACCCTCAGGGCACCGCCGTTCTTCACCAGCC
AGCGAACTGCGAAGCCAGGCCTGTTCTTCCCTCCTGGTGGCGAGGCTGAGCGCC
GCATCTCGTTCTTTGCCTCATCGCTGACGACCGCGCTCCCGGAGCCTCTGCCGAT
CGACGCCATGCCCACCTTCACCGTGCTCGTTCCCCATTACTCCGAGAAGATTCTGC
TCAGTCTGCGCGAGATTATCCGCGAGGAGGACCAGAACACCCGCGTTACCTTACT
GGAGTACCTCAAGCAGCTCCACCCTGTCGAATGGGACAATTTCGTCAAGGACACCA
AGATCTTGGCGGAAGAGTCGGGAGACGTCCAGGACGAGAAGCGCGCGCGCACGG
ACGACTTGCCGTTCTATTGCATCGGGTTCAAGACCTCGTCACCAGAGTACACCCTG
CGTACGCGTATCTGGGCCTCACTGCGCGCACAGACGCTGTACCGCACGGTCTCCG
GTATGATGAACTACTCCAAGGCGATTAAGCTCCTCTATCGCGTCGAGAACCCGGAT
GTCGTTCATGCCTTCGGTGGGAACACGGAACGTCTTGAACGCGAGCTTGAGCGCA
TGTCTCGCCGCAAGTTCAAGTTCGTCATCTCGATGCAGCGGTACTCCAAGTTCAAC
AAGGAGGAGCAGGAGAACGCCGAGTTCCTTCTGCGCGCGTACCCGGATTTGCAGA
TCGCGTACCTCGATGAAGAGCCCGGTCCCAGCAAGAGCGACGAGGTTCGGTTGTT
TTCGACACTCATCGACGGACACTCCGAGGTGGACGAGAAGACGGGCCGCCGCAA
GCCCAAGTTCCGCATCGAGCTGCCCGGTAACCCCATCCTCGGTGACGGGAAGTCG
GATAACCAGAACCACGCCATCGTCTTCTACCGCGGCGAGTACATTCAGGTCATTGA
CGCTAACCAGGACAATTACCTGGAAGAGTGTCTCAAGATCCGTAATGTCCTGGGCG
AGTTTGAGGAATACTCCGTGTCGAGCCAGAGCCCGTACGCGCAGTGGGGCCACAA
GGAGTTCAACAAGTGCCCCGTCGCTATCCTGGGTTCCCGCGAGTACATCTTCTCG
GAGAACATCGGTATCCTCGGTGACATCGCTGCCGGCAAGGAACAGACGTTCGGTA
CCATTACGGCGCGTGCGCTTGCGTGGATCGGCGGCAAGCTGCATTACGGTCACCC
GGATTTCCTCAATGCGACGTTCATGACGACGCGTGGTGGCGTGTCAAAAGCGCAG
AAGGGCTTGCATCTTAACGAGGATATCTTCGCTGGTATGACCGCCGTGTCCCGCG
GAGGGCGCATCAAGCACATGGAGTACTACCAGTGCGGCAAAGGTCGTGATCTCGG
ATTCGGCACGATCTTGAACTTCCAGACCAAGATCGGTACTGGTATGGGCGAGCAG
CTGCTCTCGCGCGAGTACTACTATCTGGGCACGCAATTGCCTATCGACCGGTTCTT
GACGTTCTACTACGCGCACGCTGGTTTCCATGTCAACAACATCCTGGTCATCTACT
CCATCCAGGTCTTCATGGTCACCCTGCTGTACCTGGGCACATTGAACAAGCAGCTG
TTCATCTGCAAGGTCAACTCCAATGGCCAGGTTCTTAGTGGACAAGCTGGGTGCTA
CAACCTCATCCCGGTCTTCGAGTGGATTCGCCGGAGTATCATCTCCATCTTCTTGG
TGTTCTTCATCGCCTTCTTGCCGTTGTTCTTGCAAGAGCTTTGCGAACGCGGAACA
GGAAAGGCGTTGCTGCGTCTCGGGAAGCACTTCCTGTCACTGTCGCCCATCTTCG
AAGTGTTCTCCACCCAAATCTACTCGCAGGCGCTCTTGAACAACATGAGTTTCGGT
GGTGCGCGCTACATCGCTACAGGACGCGGTTTCGCGACGAGTCGGATACCCTTCA
ACATCCTCTACTCGCGTTTCGCGCCGCCGAGCATCTACATGGGCATGCGTAATCTG
CTGCTCTTGCTGTACGCGACGATGGCCATTTGGATCCCACACCTGATCTACTTCTG
GTTCTCCGTCCTCTCCCTCTGCATCGCGCCATTCATGTTCAATCCGCATCAATTCTC
GTACGCTGACTTCATCATCGACTACCGGGAGTTCTTGCGCTGGATGTCGCGCGGT
AACTCGCGGACGAAGGCGAGTAGCTGGTACGGATATTGCCGTCTGTCGCGTACCG
CGATTACTGGGTACAAGAAGAAGAAACTGGGACACCCGTCGGAGAAGCTGTCGGG
CGATGTGCCGCGTGCGCCGTGGAGGAACGTCATCTTCTCGGAGATCCTTTGGCCC
ATCGGCGCGTGCATCATCTTCATCGTCGCGTACATGTTCGTCAAATCGTTCCCTGA
CGAGCAGGGCAACGCGCCGCCGAGCCCGCTGGTCCGCATTCTGCTCATCGCGGT
TGGCCCTACTGTGTGGAACGCGGCGGTGCTCATCACGCTGTTCTTCCTGTCGCTCT
TCCTGGGCCCGATGATGGATGGCTGGGTCAAGTTCGGCTCAGTCATGGCGGCACT
TGCGCATGGTCTAGCGCTCATAGGCATGCTCACGTTCTTCGAGTTCTTCTGGTTCC
TCGAGCTCTGGGATGCCTCGCACGCCGTGCTCGGCGTCATCGCCATTATTGCCGT
TCAGCGCGGGATCCAGAAGATCCTCATTGCCGTCTTCCTGACGCGTGAGTACAAG
CACGACGAGACGAACCGCGCGTGGTGGACAGGTAAATGGTATGGACGCGGGCTG
GGTACCTCGGCCATGTCCCAGCCGGCGCGCGAGTTCATCGTGAAGATCGTGGAGA
TGTCGCTGTGGACGTCGGACTTCCTGCTTGCGCACCTGTTGCTCATCATCTTGACG
GTGCCGCTACTGCTGCCGTTCTTCAACTCGATCCATTCGACGATGCTTTTCTGGTT
GCGCCCTTCGAAGCAGATTAGGCAACCTCTGTTCTCCACTAAGCAGAAGCGGCAA
CGGCGATGGATTGTCATGAAGTATACCGTGGTATATCTCGTGGTGGTGGCTTTCCT
CGTTGCGCTCATCGCTCTGCCCGCGCTCTTCCGCGAGAGCATCCACTTCAACTGC
GAGATCTGCCAGAGTATATAG
SEQ ID NO: 6
polypeptide sequence 1,3-B-D-glucan synthase I of
S. commune strain Lu15531
amino acid
S. commune
MSGPGYGRNPFDNPPPNRGPYGQQPGFPGPGPRPYDSDADMSQTYGSTTRLAGSA
GYSDRNGSFDGDRSYAPSIDSRASVPSISPFADPGIGSNEPYPAWSVERQIPMSTEEIE
DIFLDLTQKFGFQRDSMRNTFDFMMHLLDSRASRMTPNQALLTLHADYIGGQHANYRK
WYFAAQLNLDDAVGQTNNPGIQRLKTIKGATKTKSLDSALNRWRNAMNNMSQYDRLR
QIALYLLCWGEAGNIRLAPECLCFIFKCADDYYRSPECQNRMDPVPEGLYLQTVIKPLY
RFLRDQAYEVVDGKQVKREKDHDQIIGYDDVNQLFVVYPEGLAKIVMSDNTRLVDVPPA
QRFMKFAKIEWNRVFFKTYFEKRSTAHLLVNFNRIWILHVSMYFFYTAFNSPRVYAPHG
KLDPSPEMTVVSATALGGAVSTMIMILATIAEYTYIPTTANNNASHLTTRLIFLLVILALTAGP
TFYIAMIDGRTDIGQVPLIVAIVQFFISVVATLAFATIPSGRMFGDRVAGKSRKHMASQTF
TASYPSMKRSSRVASIMLWLLVFGCKYVESYFFLTSSFSSPIAVMARTKVQGCNDRIFG
SQLCTNQVPFALAIMYVMDLVLFFLDTYLWYIIWLVIFSMVRAFKLGISIWTPWSEIFTRM
PKRIYAKLLATAEMEVKYKPKVLVSQIWNAVIISMYREHLLSIEHVQRLLYHQVDGPDGR
RTLRAPPFFTSQRTAKPGLFFPPGGEAERRISFFASSLTTALPEPLPIDAMPTFTVLVPH
YSEKILLSLREIIREEDQNTRVTLLEYLKQLHPVEWDNFVKDTKILAEESGDVQDEKRAR
TDDLPFYCIGFKTSSPEYTLRTRIWASLRAQTLYRTVSGMMNYSKAIKLLYRVENPDVV
HAFGGNTERLERELERMSRRKFKFVISMQRYSKFNKEEQENAEFLLRAYPDLQIAYLDE
EPGPSKSDEVRLFSTLIDGHSEVDEKTGRRKPKFRIELPGNPILGDGKSDNQNHAIVFY
RGEYIQVIDANQDNYLEECLKIRNVLGEFEEYSVSSQSPYAQWGHKEFNKCPVAILGSR
EYIFSENIGILGDIAAGKEQTFGTITARALAWIGGKLHYGHPDFLNATFMTTRGGVSKAQ
KGLHLNEDIFAGMTAVSRGGRIKHMEYYQCGKGRDLGFGTILNFQTKIGTGMGEQLLS
REYYYLGTQLPIDRFLTFYYAHAGFHVNNILVIYSIQVFMVTLLYLGTLNKQLFICKVNSN
GQVLSGQAGCYNLIPVFEWIRRSIISIFLvFFIAFLPLFLQELCERGIGKALLRLGKHFLSL
SPIFEVFSTQIYSQALLNNMSFGGARYIATGRGFATSRIPFNILYSRFAPPSIYMGMRNLL
LLLYATMAIWIPHLIYFWFSVLSLCIAPFMFNPHQFSYADFIIDYREFLRWMSRGNSRTK
ASSWYGYCRLSRTAITGYKKKKLGHPSEKLSGDVPRAPWRNVIFSEILWPIGACIIFIVAY
MFVKSFPDEQGNAPPSPLVRILLIAVGPTVWNAAVLITLFFLSLFLGPMMDGVVVKFGSV
MAALAHGLALIGMLTFFEFFWFLELWDASHAVLGVIAIIAVQRGIQKILlAVFLTREYKHDE
TNRAWWTGKWYGRGLGTSAMSQPAREFIVKIVEMSLWTSDFLLAHLLLIILTVPLLLPFF
NSIHSTMLFWLRPSKQIRQPLFSTKQKRQRRWIVMKYTVVYLVVVAFLVALIALPALFRE
SIHFNCEICQSI
SEQ ID NO: 7
cDNA 1,3-β-D-glucan synthase II of S. commune strain Lu15531
DNA
S. commune
ATGCGGAACATGTTCGACTTCACCATGCAGCTGCTTGACAGCCGAGCGTCTCGTAT
GACCCCCAACCAGGCGCTCCTCACCCTCCACGCCGACTACATTGGTGGCCAGCAT
GCGAACTACCGGAAGTGGTACTTCGCGGCGCAGCTCGACCTTGACGACGCCGTG
GGACAAACTCAGAATCCGGGTCTCAACCGCCTCAAGTCCACTCGCGGATCGGGCA
AGCGACCACGCCATGAAAAGTCGCTGAACACGGCATTGGAGCGCTGGCGGCAAG
CCATGAACAACATGTCGCAGTATGACCGCTTACGCCAGATCGCGCTCTACCTGCTC
TGCTGGGGCGAAGCGGCGCAAGTGCGATTCATGCCCGAGTGCTTGTGCTTCATCT
TCAAGTGCGCCGACGACTATTATCGTTCGCCGGAGTGCCAGAACAGGATGGAGCC
GGTACCGGAGGGTCTCTACCTGAGGACGGTCGTAAAGCCGCTCTACAGATTTGTC
CGGGATCAAGGCTATGAGGTGGTGGAGGGAAAATTCGTACGGCGGGAACGGGAT
CACGACCAAATCATTGGTTACGATGACGTGAATCAGCTGTTCTGGTACCCGGAGGG
CATTGCCCGTATCGTCCTGTCGGACAAGAGTCGTCTGGTCGACCTCCCTCCAGCA
CAGCGCTTCATGAAGTTCGACCGTATCGAGTGGAATCGCGTCTTCTTCAAGACGTT
CTACGAGACTCGATCCTTTACGCATCTTTTGGTCGACTTCAACCGTATCTGGGTCGT
GCACATCGCTCTCTACTTCTTCTACACCGCATACAACTCCCCCACGATCTACGCCAT
CAACGGCAACACTCCGACGTCTCTGGCTTGGAGCGCGACTGCGCTCGGCGGTGC
GGTAGCGACAGGTATCATGATCCTCGCCACGATCGCCGAGTTCTCGCACATCCCC
ACGACATGGAACAACACCTCGCATCTGACTCGCCGCCTCGCCTTCCTCCTCGTCAC
GCTCGGCCTCACATGTGGTCCGACGTTCTACGTCGCGATTGCAGAGAGCAACGGG
AGCGGCGGCTCTTTGGCCTTGATTCTCGGCATCGTCCAGTTCTTCATCTCCGTCGT
AGCGACTGCGCTCTTCACTATCATGCCTTCTGGTCGTATGTTCGGCGACCGCGTCG
CAGGCAAGAGTCGCAAGTATCTCGCCAGCCAGACGTTCACGGCCAGCTACCCGTC
GTTGCCCAAGCACCAGCGGTTCGCATCACTCCTGATGTGGTTCCTCATCTTCGGGT
GCAAGTTGACGGAGAGTTACTTCTTCCTGACGTTGTCCTTCCGCGACCCTATTCGC
GTCATGGTCGGCATGAAGATCCAGAACTGCGAGGACAAGATTTTCGGCAGCGGCC
TTTGCAGGAATCACGCAGCATTCACCCTCACGATCATGTACATCATGGACCTCGTC
TTGTTCTTCCTCGACACCTTCCTTTGGTATGTCATCTGGAACTCGGTTTTCAGTATC
GCACGCTCTTTCGTACTCGGCCTTTCGATCTGGACACCATGGAGGGACATCTTCCA
GCGTCTGCCGAAGCGTATCTACGCGAAGCTTCTAGCGACCGGCGACATGGAGGTC
AAGTACAAGCCCAAGGTCTTGGTTTCGCAAATCTGGAACGCCATCATCATCTCCAT
GTACCGCGAGCACTTGCTCTCTATCGAGCACGTTCAAAAGCTCCTGTACCATCAAG
TGGACACTGGCGAAGCCGGCAAGCGGAGTCTTCGCGCGCCTCCGTTCTTCGTCGC
GCAGGGCAGCAGCGGTGGCTCGGGCGAGTTCTTCCCGCCTGGTAGCGAGGCTGA
GCGTCGTATCTCTTTCTTCGCGCAGTCTCTATCTACGGAGATTCCTCAGCCCATCC
CGGTTGACGCCATGCCGACGTTCACAGTGCTTACGCCTCACTACAGCGAGAAGAT
CCTTCTTTCGCTCCGTGAGATTATCCGCGAGGAGGACCAGAACACCCGCGTGACA
TTGCTTGAGTATCTCAAGCAGCTTCACCCGGTCGAGTGGGAGAACTTCGTCAAGGA
CACCAAGATTTTGGCCGAGGAGTCCGCTATGTTCAACGGTCCAAGTCCTTTCGGCA
ACGATGAGAAGGGTCAGTCCAAGATGGACGATCTTCCTTTCTACTGCATCGGTTTC
AAGAGCGCCGCGCCCGAGTACACCCTCCGCACCCGTATCTGGGCGTCCTTGCGC
GCGCAGACCCTCTACCGCACGGTCTCCGGCATGATGAACTATGCGAAGGCGATTA
AGCTGCTCTACCGCGTCGAGAACCCCGAGGTCGTGCAGCAGTTCGGCGGTAACAC
GGACAAGCTCGAGCGCGAGTTGGAGCGGATGGCCCGGCGGAAGTTCAAGTTCCT
GGTGTCCATGCAGCGCTACTCGAAGTTCAACAAGGAGGAGCACGAGAACGCCGAG
TTCTTGCTCCGCGCGTACCCGGACCTGCAGATCGCGTACCTGGAGGAAGAGCCTC
CTCGCAAGGAGGGTGGCGATCCACGCATCTTCTCTGCCCTCGTCGACGGCCACAG
CGACATCATCCCGGAGACCGGCAAGCGGCGCCCCAAGTTCCGCATCGAGCTGCC
CGGCAACCCCATTCTCGGTGACGGCAAGTCGGACAACCAGAACCACGCCATCGTC
TTCTACCGCGGCGAGTACCTCCAGCTTATCGACGCCAACCAGGACAACTACCTCGA
GGAGTGCTTGAAGATCCGTAACGTACTCGCCGAGTTCGAGGAGTACGACGTCTCT
AGCCAGAGTCCGTACGCGCAGTGGAGTGTCAAGGAGTTCAAGCGCTCCCCGGTCG
CCATCGTCGGTGCACGCGAGTATATCTTCTCGGAGCACATCGGTATTCTCGGTGAT
TTGGCGGCTGGCAAGGAACAGACGTTCGGTACGCTCACGGCACGCAACAACGCCT
TCCTTGGCGGCAAGCTGCACTACGGTCACCCGGATTTCCTCAACGCCCTCTACATG
AACACGCGCGGTGGTGTCTCCAAGGCGCAGAAGGGTCTCCATCTCAACGAGGATA
TTTACGCCGGTATGAACGCGGTCGGTCGCGGTGGACGCATCAAGCATAGCGAATA
CTACCAGTGCGGCAAGGGTCGTGACCTCGGTTTTGGCACCATCTTGAACTTCCAGA
CCAAGATCGGTACGGGTATGGGCGAGCAGATCCTCTCGCGCGAGTACTACTACCT
CGGAACCCAATTGCCCATCGATCGCTTCCTCACGTTCTACTACGCGCACCCAGGTT
TCCAGATCAACAACATGCTGGTTATCCTATCCGTGCAGGTCTTCATCGTTACCATGG
TCTTCCTCGGTACCTTGAAGTCTTCGGTCACGATCTGCAAGTACACGTCCAGCGGT
CAGTACATCGGTGGTCAATCCGGTTGCTACAACCTCGTCCCGGTCTTCCAGTGGAT
CGAGCGCTGCATCATCAGCATCTTCTTGGTGTTCATGATCGCTTTCATGCCGCTCTT
CCTGCAAGAACTCGTCGAGCGCGGTACCTGGAGTGCCATCTGGCGTCTGCTCAAG
CAGTTTATGTCGCTGTCGCCTGTCTTCGAGGTGTTCTCCACCCAGATTCAGACACA
CTCCGTGTTGAGCAACTTGACGTTCGGTGGTGCGCGTTACATCGCTACCGGTCGT
GGGTTCGCCACCAGTCGTATCAGCTTCAGCATCTTGTTCTCGCGTTTCGCAGGCCC
GAGTATCTACCTCGGCATGCGCACGCTCATTATGCTGCTCTACGTGACGTTGACGA
TCTGGACGCCATGGGTCATTTACTTCTGGGTTTCCATTCTCTCGCTCTGCATCGCG
CCGTTCTTGTTCAATCCGCATCAATTCGTCTTCTCGGATTTCCTCATCGACTACAGG
GAATACCTCCGGTGGATGTCGCGTGGTAACTCGCGCTCGCACAACAACTCCTGGA
TTGGGTACTGCCGGTTGTCCCGCACGATGATCACTGGGTACAAGAAGAAGAAGCT
GGGCCACCCGTCGGAGAAGCTTTCCGGCGACGTTCCTCGTGCAGGCTGGCGCGC
CGTCTTATTCTCGGAGATCATCTTCCCGGCATGCATGGCCATCCTCTTCATCATCG
CGTACATGTTCGTCAAGTCGTTCCCTCTCGACGGCAAGCAGCCTCCCTCCGGCCT
CGTTCGCATCGCCGTCGTGTCTATCGGCCCCATCGTGTGGAACGCCGCCATCCTG
TTGACGCTCTTCCTTGTGTCGTTGTTCCTCGGCCCCATGCTCGACCCGGTCTTCCC
CCTCTTCGGTTCCGTTATGGCCTTCATCGCGCATTTCCTCGGCACAATCGGAATGA
TTGGGTTCTTCGAGTTCCTGTGGTTCCTCGAGTCCTGGGAGGCGTCGCATGCCGT
GCTGGGTCTCATCGCCGTCATCTCCATCCAGCGCGCCATTCACAAAATTCTTATCG
CCGTTTTCCTCAGTCGCGAGTTCAAGCACGACGAGACGAACAGGGCTTGGTGGAC
TGGTCGCTGGTATGGCCGTGGCCTCGGCACGCACGCCATGTCGCAGCCGGCGCG
TGAGTTCGTCGTCAAGATCATCGAGTTGTCGCTCTGGAGCTCGGATCTCATACTCG
GCCACATCCTGCTGTTCATGCTTACTCCGGCTGTCCTCATCCCGTACTTCGACCGT
CTGCACGCCATGATGCTCTTCTGGCTGCGCCCCTCAAAGCAAATCCGCGCGCCTC
TGTACTCAATCAAGCAGAAGAGGCAAAGACGCTGGATTATCATGAAGTACGGTACT
GTATACGTTACCGTCATCGCGATCTTCGTCGCGCTCATCGCGCTTCCCCTCGTCTT
CCGACACACTCTAAAGGTCGAGTGCTCCCTTTGCGACAGCTTGTAA
SEQ ID NO: 8
polypeptide sequence 1,3-β-D-glucan synthase II
of S. commune strain Lu15531
amino acid
S. commune
MRNMFDFTMQLLDSRASRMTPNQALLTLHADYIGGQHANYRKWYFAAQLDLDDAVGQ
TQNPGLNRLKSTRGSGKRPRHEKSLNTALERWRQAMNNMSQYDRLRQIALYLLCWG
EAAQVRFMPECLCFIFKCADDYYRSPECQNRMEPVPEGLYLRTVVKPLYRFVRDQGY
EVVEGKFVRRERDHDQIIGYDDVNQLFWYPEGIARIVLSDKSRLVDLPPAQRFMKFDRI
EWNRVFFKTFYETRSFTHLLVDFNRIWVVHIALYFFYTAYNSPTIYAINGNTPTSLAWSA
TALGGAVATGIMILATIAEFSHIPTTWNNTSHLTRRLAFLLVTLGLTCGPTFYVAIAESNG
SGGSLALILGIVQFFISVVATALFTIMPSGRMFGDRVAGKSRKYLASQTFTASYPSLPKH
QRFASLLMWFLIFGCKLTESYFFLTLSFRDPIRVMVGMKIQNCEDKIFGSGLCRNHAAFT
LTIMYIMDLVLFFLDTFLWYVIWNSVFSIARSFVLGLSIWTPWRDIFQRLPKRIYAKLLATG
DMEVKYKPKVLVSQIWNAIIISMYREHLLSIEHVQKLLYHQVDTGEAGKRSLRAPPFFVA
QGSSGGSGEFFPPGSEAERRISFFAQSLSTEIPQPIPVDAMPTFTVLTPHYSEKILLSLR
EIIREEDQNTRVTLLEYLKQLHPVEWENFVKDTKILAEESAMFNGPSPFGNDEKGQSKM
DDLPFYCIGFKSAAPEYTLRTRIWASLRAQTLYRTVSGMMNYAKAIKLLYRVENPEVVQ
QFGGNTDKLERELERMARRKFKFLVSMQRYSKFNKEEHENAEFLLRAYPDLQIAYLEE
EPPRKEGGDPRIFSALVDGHSDIIPETGKRRPKFRIELPGNPILGDGKSDNQNHAIVFYR
GEYLQLIDANQDNYLEECLKIRNVLAEFEEYDVSSQSPYAQWSVKEFKRSPVAIVGARE
YIFSEHIGILGDLAAGKEQTFGTLTARNNAFLGGKLHYGHPDFLNALYMNTRGGVSKAQ
KGLHLNEDIYAGMNAVGRGGRIKHSEYYQCGKGRDLGFGTILNFQTKIGTGMGEQILS
REYYYLGTQLPIDRFLTFYYAHPGFQINNMLVILSVQVFIVTMVFLGTLKSSVTICKYTSS
GQYIGGQSGCYNLVPVFQWIERCIISIFLVFMIAFMPLFLQELVERGTWSAIWRLLKQFM
SLSPVFEVFSTQIQTHSVLSNLTFGGARYIATGRGFATSRISFSILFSRFAGPSIYLGMRT
LIMLLYVTLTIWTPWVIYFWVSILSLCIAPFLFNPHQFVFSDFLIDYREYLRWMSRGNSRS
HNNSWIGYCRLSRTMITGYKKKKLGHPSEKLSGDVPRAGWRAVLFSEIIFPACMAILFIIA
YMFVKSFPLDGKQPPSGLVRIAVVSIGPIVWNAAILLTLFLVSLFLGPMLDPVFPLFGSV
MAFIAHFLGTIGMIGFFEFLWFLESWEASHAVLGLIAVISIQRAIHKILIAVFLSREFKHDET
NRAWWTGRWYGRGLGTHAMSQPAREFVVKIIELSLWSSDLILGHILLFMLTPAVLIPYF
DRLHAMMLFWLRPSKQIRAPLYSIKQKRQRRWIIMKYGTVYVTVIAIFVALIALPLVFRFHT
LKVECSLCDSL
SEQ ID NO: 9
Gene sequence 1,3-β-D-glucan synthase I of S. commune
strain Lu15634
DNA
S. commune
CCCGTCCCTCAAGGCCGTTCTTTCGCTGGCGACCGACCCGGTGTTCGCGAGAACC
TGTTGTTTCTGACGATCATCAACCCTTTCTTCTCGTCGCTCTTTAGCTCTCCCTAGA
CCGTCTTTTACTCTACTCTTCGACGCACGCCATGTCCGGTCCAGGATATGGCAGGA
ATCCATTCGACAATCCCCCGCCCAACAGAGGTCCCTATGGCCAGCAGCCAGGTTT
CCCGGGGCCCGGCCCTCGGCCTTACGACTCGGACGCGGACATGAGCCAGACCTA
TGGCAGCACAACCAGGCTCGCCGGCAGTGCCGGTTACAGCGACAGAAACGgtgcga
acgtcgctaccgtacttcctcgatcgtcgactcacatatcacgcagGCAGCTTCGACGGCGACCGCTCCT
ACGCGCCCTCAATTGACTCGCGCGCCAGCGTGCCCAGCATATCGCCCTTCGCAGA
CCCGGGTATCGGCTCTAATGAGCCGTATCCCGCTTGGTCGGTCGAACGCCAGATC
CCCATGTCCACGGAGGAGATTGAGGATATCTTCCTCGACCTCACCCAAAAGTTTGG
CTTCCAGCGCGACTCCATGCGGAATACGgtgcgtgaataagcagcccactcgaccgcgggaacagc
tcaattgacctgtcacccagTTCGACTTCATGATGCACCTCCTTGATTCCCGTGCCTCGCGCA
TGACGCCCAACCAAGCTCTGCTCACGCTTCACGCCGACTACATTGGTGGCCAGCA
CGCCAACTATAGGAAGTGGTATTTCGCCGCTCAGCTCAACCTCGATGACGCGGTC
GGGCAAACCAATAACCCCGGTATCCAGCGCTTGAAGACCATCAAGGGCGCTACGA
AGACCAAGTCGCTCGACAGCGCACTCAACCGCTGGCGCAATGCGATGAACAACAT
GAGCCAGTACGATCGCCTCCGGCAAATTGCGCTCTATCTCCTCTGCTGGGGAGAA
GCAGGCAACATCCGTCTGGCGCCCGAGTGCTTGTGCTTCATCTTCAAGTGCGCGG
ACGACTACTACAGAAGTCCCGAGTGTCAGAACCGGATGGACCCCGTGCCGGAAGG
GCTGTACCTCCAGACGGTCATCAAGCCGCTCTATCGCTTCCTACGTGATCAGGCGT
ACGAAGTCGTTGATGGGAAGCAAGTGAAGCGCGAGAAGGACCACGACCAGATTAT
CGGTTATGACGACGTCAACCAGTTATTCTGGTATCCGGAAGGTTTGGCTAAGATCG
TCATGTCGGACAACgtgcgtatgatcttatcggttacaattcgtccgctcacatctttccagACACGACTTGTA
GATGTACCTCCGGCGCAGCGGTTCATGAAGTTCGCCAAGATCGAGTGGAACCGCG
TCTTCTTCAAGACGTACTTTGAGAAGCGCTCTACTGCCCATCTCCTGGTCAACTTCA
ACCGTATATGGATCCTCCACGTCTCGATGTACTTCTTCTACACGGCATTCAACTCTC
CACGAGTCTACGCGCCGCACGGCAAACTCGACCCCTCCCCTGAGATGACCTGGTC
CGCGACTGCCCTTGGAGGCGCTGTGTCCACCATGATCATGATCCTTGCCACTATCG
CGGAGTACACCTACATCCCCACGACATGGAACAATGCGTCGCACCTCACCACGCG
GCTCATTTTCCTCCTGGTCATCCTCGCGCTCACTGCTGGACCAACATTCTATATCGC
CATGATAGACGGACGCACGGACATCGGCCAAGTACCACTCATCGTGGCCATAGTG
CAGTTCTTCATCTCCGTCGTCGCCACCCTCGCTTTCGCTACCATCCCTTCTGGTCG
CATGTTCGGCGACCGTGTGGCTGGCAAGTCAAGAAAGCACATGGCATCGCAGACG
TTCACAGCGTCGTACCCGTCCATGAAGCGGTCATCTCGCGTAGCGAGTATCATGCT
GTGGCTTTTGGTCTTTGGCTGCAAATACGTCGAGTCTTACTTCTTCTTGACGTCCTC
CTTCTCCAGCCCGATCGCGGTCATGGCGCGTACGAAGGTACAGGGCTGCAACGAC
CGTATCTTCGGCAGCCAGCTGTGCACGAATCAGGTCCCGTTCGCGCTGGCAATCA
TGTACGTGATGGACCTGGTACTGTTCTTCCTGGACACGTACCTGTGGTACATCATC
TGGCTGGTGATCTTCTCGATGGTGCGCGCGTTCAAGCTTGGTATCTCGATCTGGAC
GCCCTGGAGCGAGATCTTCACCCGCATGCCGAAGCGTATCTACGCGAAGCTGCTG
GCGACGGCCGAGATGGAGGTCAAGTATAAGCCCAAGgtatgctgaatgcaatctggtcaggtga
attcaccctcatattgttgtgcagGTGCTCGTCTCGCAAATCTGGAACGCGGTCATCATCTCCAT
GTACCGGGAGCATCTCTTGTCCATCGAGCACGTCCAGCGCCTGCTATACCACCAG
GTTGATGGTCCAGACGGTCGCCGCACCCTCAGGGCACCGCCGTTCTTCACCAGCC
AGCGAACTGCGAAGCCAGGCCTGTTCTTCCCTCCTGGTGGCGAGGCTGAGCGCC
GTATCTCGTTCTTTGCCTCATCGCTGACGACCGCGCTCCCTGAGCCTCTGCCGATC
GACGCCATGCCCACCTTCACCGTGCTCGTTCCCCATTACTCGGAGAAGATTCTGCT
CAGTCTGCGCGAGATTATTCGCGAGGAGGACCAGAACACCCGCGTCACCTTGCTG
GAGTACCTCAAGCAGCTCCACCCTGTCGAATGGGACAACTTCGTCAAGGACACCAA
GATCTTGGCGGAAGAGTCGGGCGACGTCCAGGACGAGAAGCGCGCGCGCACGGA
CGACTTGCCGTTCTACTGCATCGGGTTCAAGACCTCGTCACCAGAGTACACCCTGC
GTACGCGTATCTGGGCTTCACTGCGCGCACAGACGCTGTACCGCACGGTCTCCGG
TATGATGAACTACTCCAAGGCGATCAAGCTCCTCTATCGCGTCGAGAACCCGGATG
TCGTTCATGCCTTCGGTGGGAACACGGAACGTCTTGAACGCGAGCTTGAGCGCAT
GTCTCGCCGCAAGTTCAAGTTCGTCATCTCGATGCAGCGGTACTCTAAGTTCAACA
AGGAGGAGCAAGAGAACGCCGAATTCCTTCTGCGCGCGTACCCGGATTTGCAGAT
CGCGTACCTCGATGAAGAGCCCGGTCCCAGCAAGAGCGACGAGGTTCGGTTGTTT
TCGACACTCATCGATGGACACTCCGAGGTGGATGAGAAGACCGGCCGCCGCAAGC
CCAAGTTCCGCATTGAGCTGCCCGGTAACCCCATCCTCGGTGACGGGAAGTCGGA
TAACCAGAACCACGCCATTGTCTTCTACCGCGGCGAGTACATCCAGGTCATCGACG
CTAACCAGGACAATTACCTGGAAGAGTGTCTCAAGATCCGTAACGTCCTGGGCGAG
TTTGAGGAATACTCCGTGTCGAGCCAGAGCCCGTACGCACAGTGGGGCCACAAGG
AGTTCAACAAGTGCCCCGTCGCTATCCTGGGTTCTCGCGAGTACATCTTCTCGGAG
AACATCGGTATCCTCGGTGACATCGCCGCCGGCAAGGAACAGACGTTCGGTACCA
TTACGGCGCGTGCGCTTGCGTGGATCGGCGGCAAGCTGCATTACGGTCACCCGGA
TTTCCTCAATGCGACGTTCATGACGACGCGTGGTGGCGTGTCAAAAGCGCAGAAG
GGCTTGCATCTCAACGAGGATATCTTCGCTGGTATGACCGCCGTGTCCCGCGGAG
GGCGCATCAAGCACATGGAGTACTACCAGTGCGGCAAAGGTCGTGATCTCGGTTT
CGGCACGATCTTGAACTTCCAGACGAAGATCGGTACTGGTATGGGCGAGCAGCTC
CTCTCGCGCGAGTACTACTACCTGGGCACGCAATTGCCTATCGACCGGTTCTTGAC
GTTCTACTACGCGCACGCTGGTTTCCACGTCAACAACATCCTGGTCATCTACTCCA
TCCAGGTCTTCATGGTCACCTgtaagtgcaggcgctcatgaccgccgagaacgtagtctgacggatgtgca
gTGCTGTACCTGGGCACATTGAACAAGCAGCTGTTCATCTGCAAGGTCAACTCCAA
TGGCCAGGTTCTTAGTGGACAAGCTGGGTGCTACAACCTCATCCCGGTCTTCGAGT
GGATTCGCCGGAGTATCATCTCCATCTTCTTGGTGTTCTTCATCGCCTTCTTGCCTC
TATTCTTGCAAGgtatgttcactttccatgtgtcatccgttagccgctcaccatacgacagAGCTGTGCGAGC
GCGGAACGGGAAAGGCGTTGCTGCGTCTCGGGAAGCACTTCTTGTCACTGTCGCC
CATTTTCGAAGTGTTCTCCACCCAGATTTACTCGCAGGCGCTCTTGAACAACATGA
GCTTCGGTGGTGCGCGCTACATCGCCACAGGTCGTGGTTTCGCGACTAGTCGCAT
ACCCTTCAACATCCTCTACTCGCGTTTCGCGCCGCCAAGCATCTACATGGGCATGC
GTAACCTGCTGCTCCTGCTGTACGCGACGATGGCCATTTGGATCCCGCACCTGATC
TACTTCTGGTTCTCCGTCCTCTCCCTCTGCATCGCGCCATTCATGTTCAATCCGCAT
CAATTCTCGTACGCCGACTTCATCATCGACTACCGGGAGTTCTTGCGCTGGATGTC
GCGCGGTAACTCGCGAACGAAGGCGAGCAGCTGGTACGGATACTGCCGTCTGTC
GCGTACCGCGATTACTGGGTACAAGAAGAAGAAGCTGGGACACCCGTCGGAGAAG
CTGTCGGGCGACGTACCGCGTGCGCCGTGGAGGAACGTTATCTTCTCGGAGATCC
TGTGGCCCATCGGCGCGTGCATCATCTTCATCGTCGCGTACATGTTCGTCAAGTCG
TTCCCCGACGAGCAGGGCAACGCGCCGCCGAGCCCGCTGGTCCGGATTCTGCTC
ATCGCGGTTGGCCCTACTGTGTGGAACGCGGCGGTGCTCATAACGCTGTTCTTCC
TGTCGCTCTTCCTGGGCCCGATGATGGATGGCTGGGTCAAGTTCGGCTCGGTCAT
GGCGGCCCTTGCGCATGGCCTGGCGCTTATAGGCATGCTCACGTTCTTTGAGTTCT
TCgtacgtccttcgcgttgtgtcgtcaagtgctctgctaacgccgtcttcagTGGTTCCTTGAGCTCTGGGATG
CCTCGCACGCCGTGCTCGGCGTCATCGCTATCATTGCCGTTCAGCGCGGGATCCA
GAAGATCCTCATTGCCGTCTTCCTGACGCGTGAGTACAAGCACGACGAGACGAAC
CGCGCGTGGTGGACAGGTAAATGGTATGGACGCGGGCTGGGTACCTCGGCCATG
TCCCAGCCGGCGCGCGAGTTCATCGTGAAGATCGTGGAGATGTCGTTGTGGACGT
CGGACTTCCTGCTTGCGCACCTGTTGCTCATCATCTTGACGGTGCCGCTACTGCTG
CCGTTCTTCAACTCAATTCATTCGACGATGCTTTgtgagtggtttgtagtcgttggtcatggatgatttct
gactcgcgtgcagTCTGGTTGCGCCCTTCGAAGCAGATTAGGCAACCTCTGTTCTCCACC
AAGCAGAAGCGGCAACGGCGATGGATTgtgagttcctttgattgctctgggtaccgaccttcgctcaccttt
cttagGTCATGAAGTATACCGTGGTATATCTCGTGGTGGTGGCTTTCCTCGTCGCGCT
CATCGCTCTGCgtacgttttccctcgcgctcaccctgtattttcactaacgtttectccagCCGCCCTCTTCCG
CGAGAGCATCCACTTCAACTGCGAGATCTGCCAGAGTATATAGTCATATAACGACG
TCTATCGTATCGCCGGACGAGAGCCCCGTCGCCTACACACTGACATGGAATCGCT
GTGTATACAATCGATCTTCTGACCGCGTCGGGGGCGTTGCCGTCTTTCTACTATCA
ATTTGCTTGTGTATCAACATTTCTTCTCTCCAAGCCTACATTGACATAGAGTAATAGC
CCATGTTCATACAACAATCGCATAGCATTGCATATACCAT
SEQ ID NO: 10
translation of SEQ ID NO: 13
amino acid
S. commune
MSGPGYGRNPFDNPPPNRGPYGQQPGFPGPGPRPYDSDADMSQTYGSTTRLAGSA
GYSDRNGSFDGDRSYAPSIDSRASVPSISPFADPGIGSNEPYPAWSVERQIPMSTEEIE
DIFLDLTQKFGFQRDSMRNTFDFMMHLLDSRASRMTPNQALLTLHADYIGGQHANYRK
WYFAAQLNLDDAVGQTNNPGIQRLKTIKGATKTKSLDSALNRWRNAMNNMSQYDRLR
QIALYLLCWGEAGNIRLAPECLCFIFKCADDYYRSPECQNRMDPVPEGLYLQTVIKPLY
RFLRDQAYEVVDGKQVKREKDHDQIIGYDDVNQLFWYPEGLAKIVMSDNTRLVDVPPA
QRFMKFAKIEWNRVFFKTYFEKRSTAHLLVNFNRIWILHVSMYFFYTAFNSPRVYAPHG
KLDPSPEMTWSATALGGAVSTMIMILATIAEYTYIPTTWNNASHLTTRLIFLLVILALTAGP
TFYIAMIDGRTDIGQVPLIVAIVQFFISVVATLAFATIPSGRMFGDRVAGKSRKHMASQTF
TASYPSMKRSSRVASIMLWLLVFGCKYVESYFFLTSSFSSPIAVMARTKVQGCNDRIFG
SQLCTNQVPFALAIMYVMDLVLFFLDTYLWYIIWLVIFSMVRAFKLGISIWTPWSEIFTRM
PKRIYAKLLATAEMEVKYKPKVLVSQIWNAVIISMYREHLLSIEHVQRLLYHQVDGPDGR
RTLRAPPFFTSQRTAKPGLFFPPGGEAERRISFFASSLTTALPEPLPIDAMPTFTVLVPH
YSEKILLSLREIIREEDQNTRVTLLEYLKQLHPVEWDNFVKDTKILAEESGDVQDEKRAR
TDDLPFYCIGFKTSSPEYTLRTRIWASLRAQTLYRTVSGMMNYSKAIKLLYRVENPDVV
HAFGGNTERLERELERMSRRKFKFVISMQRYSKFNKEEQENAEFLLRAYPDLQIAYLDE
EPGPSKSDEVRLFSTLIDGHSEVDEKTGRRKPKFRIELPGNPILGDGKSDNQNHAIVFY
RGEYIQVIDANQDNYLEECLKIRNVLGEFEEYSVSSQSPYAQWGHKEFNKCPVAILGSR
EYIFSENIGILGDIAAGKEQTFGTITARALAWIGGKLHYGHPDFLNATFMTTRGGVSKAQ
KGLHLNEDIFAGMTAVSRGGRIKHMEYYQCGKGRDLGFGTILNFQTKIGTGMGEQLLS
REYYYLGTQLPIDRFLTFYYAHAGFHVNNILVIYSIQVFMVTLLYLGTLNKQLFICKVNSN
GQVLSGQAGCYNLIPVFEWIRRSIISIFLVFFIAFLPLFLQELCERGTGKALLRLGKHFLSL
SPIFEVFSTQIYSQALLNNMSFGGARYIATGRGFATSRIPFNILYSRFAPPSIYMGMRNLL
LLLYATMAIWIPHLIYFWFSVLSLCIAPFMFNPHQFSYADFIIDYREFLRWMSRGNSRIK
ASSWYGYCRLSRTAITGYKKKKLGHPSEKLSGDVPRAPWRNVIFSEILWPIGACIIFIVAY
MFVKSFPDEQGNAPPSPLVRILLIAVGPTVWNAAVLITLFFLSLFLGPMMDGWVKFGSV
MAALAHGLALIGMLTFFEFFWFLELWDASHAVLGVIAIIAVQRGIQKILIAVFLTRKWYGR
GLGTSAMSQPAREFIVKIVEMSLWTSDFLLAHLLLIILTVPLLLPFFNSIHSTMLFWLRPSK
QIRQPLFSTKQKRQRRWIVMKYTVVYLVVVAFLVALIALPALFRESIHFNCEICQSI
SEQ ID NO: 11
Gene sequence 1,3-β-D-glucan synthase II of
S. commune strain Lu15634
DNA
S. commune
CTGTCCAAGGAGGAGATCGAGGACATCTTCCTCGATTTGACGCAGAAGTTTGGCTT
TCAGCGGGATTCCATGCGGAATATGgtacgtggcgtgtgcccatgtgcggcgttctgaggcctaacgttttc
cgccagTTCGACTTCACCATGCAGCTGCTTGACAGCCGAGCGTCTCGTATGACCCCC
AACCAGGCGCTCCTCACCCTCCACGCCGACTACATTGGTGGCCAGCATGCGAACT
ACCGGAAGTGGTACTTCGCGGCGCAGCTCGACCTTGACGACGCCGTGGGACAAAC
TCAGAATCCGGGTCTCAACCGCCTCAAGTCCACTCGCGGATCGGGCAAGCGACCA
CGCCATGAAAAGTCGCTGAACACGGCATTGGAGCGCTGGCGGCAAGCCATGAACA
ACATGTCGCAGTATGACCGCTTACGCCAGATCGCGCTCTACCTGCTCTGCTGGGG
CGAAGCGGCGCAAGTGCGATTCATGCCCGAGTGCTTGTGCTTCATCTTCAAGTGC
GCCGACGACTACTATCGTTCGCCGGAGTGCCAGAACAGGATGGAGCCGGTACCG
GAGGGTCTCTACCTGAGGACGGTCGTAAAGCCGCTCTACAGATTTGTCCGGGATC
AAGGCTATGAGGTGGTGGAGGGAAAATTCGTACGGCGGGAACGGGATCACGACCA
AATCATTGGTTACGATGACGTGAATCAGCTGTTCTGGTACCCGGAGGGAATTGCCC
GTATCGTCCTGTCGGACAAGgtaagcacctctgtgcatcttctgtgacatacagggctaattgtcgagcagA
GTCGTCTAGTCGACCTCCCCCCAGCACAGCGCTTCATGAAGTTCGACCGTATCGA
GTGGAATCGCGTCTTCTTCAAGACGTTTTACGAGACTCGATCCTTCACGCATCTTTT
GGTCGACTTCAACCGTATCTGGGTCGTGCACATCGCTCTCTACTTCTTCTACACTG
CATACAACTCCCCCACGATCTACGCCATCAACGGCAACACACCGACGTCTCTGGCT
TGGAGCGCGACTGCGCTCGGCGGTGCGGTAGCGACAGGTATCATGATCCTCGCC
ACGATCGCCGAGTTCTCGCACATCCCCACGACATGGAACAACACCTCGCATCTGAC
TCGCCGCCTCGCCTTCCTCCTCGTCACGCTCGGCCTCACATGTGGTCCGACGTTC
TACGTCGCGATTGCAGAGAGCAACGGGAGCGGCGGCTCTTTGGCCTTGATTCTCG
GTATCGTCCAGTTCTTCATCTCCGTCGTGGCAACTGCGCTCTTCACTATCATGCCTT
CTGGTCGTATGTTCGGCGACCGTGTCGCAGGCAAGAGTCGCAAGTATCTCGCCAG
CCAGACGTTCACGGCCAGCTACCCGTCGTTGCCCAAGCACCAGCGGTTCGCCTCA
CTCCTGATGTGGTTCCTCATCTTCGGGTGCAAGTTGACGGAGAGTTACTTCTTTCT
GACGCTGTCCTTCCGCGACCCTATCCGCGTCATGGTCGGCATGAAGATCCAGAAC
TGCGAGGACAAGATTTTCGGCAGCGGCCTTTGCAGGAATCACGCAGCATTCACCC
TCACGATCATGTACATCATGGACCTCGTCTTGTTCTTCCTCGACACCTTCCTTTGGT
ATGTCATCTGGAACTCGGTTTTCAGTATCGCACGCTCTTTCGTACTCGGCCTTTCGA
TCTGGACACCGTGGAGAGACATCTTCCAGCGTCTGCCGAAGCGGATCTACGCGAA
GCTTCTGGCGACTGGCGACATGGAGGTCAAGTACAAGCCCAAGgtatgcgttgagctcgcc
gtaaatccacttaaggctaacacgttcgcagGTCTTGGTCTCGCAAATCTGGAACGCCATCATCAT
CTCCATGTACCGCGAGCACTTGCTCTCTATTGAGCACGTCCAGAAGCTCCTGTACC
ACCAAGTGGACACTGGCGAAGCCGGCAAGCGGAGTCTTCGCGCGCCTCCGTTCTT
CGTCGCGCAGGGCAGCAGCGGTGGCTCGGGCGAGTTCTTCCCGCCTGGCAGCGA
GGCCGAGCGTCGTATCTCTTTCTTCGCGCAGTCGCTTTCTACGGAGATTCCTCAGC
CCATCCCGGTCGACGCCATGCCGACGTTCACGGTGCTTACGCCTCACTACAGCGA
GAAGgtacatgctccccttgtagccatatgacatcagctgactgtcgtgcacagATCCTTCTCTCTCTCCGTG
AAATTATCCGCGAGGAGGACCAGAACACTCGCGTTACGTTGCTCGAGTACCTGAAG
CAGCTGCATCCGGTCGAGTGGGAGAATTTCGTCAAGGACACTAAAATTTTGGCCGA
GGAGTCCGCTATGTTTAACGGTCCGAGTCCTTTCGGCAACGACGAGAAGGGTCAG
TCCAAGATGGACGATCTACCGTTCTACTGCATCGGTTTCAAGAGCGCCGCGCCCG
AGTACACCCTCCGCACCCGTATCTGGGCGTCCCTGCGCGCGCAGACGCTGTACCG
CACGGTCTCCGGCATGATGAACTATGCGAAGGCGATCAAGCTGCTCTACCGCGTT
GAGAACCCGGAGGTCGTACAACAGTTCGGCGGCAACACGGACAAGCTCGAGCGC
GAGTTGGAGCGGATGGCGCGACGGAAGTTCAAGTTCCTCGTGTCCATGCAGCGCT
ACTCGAAGTTCAACAAGGAGGAGCACGAGAACGCCGAGTTCTTGCTCCGCGCGTA
CCCGGACTTGCAGATCGCGTACCTCGAGGAAGAGCCCCCTCGCAAGGAGGGCGG
CGATCCACGCATCTTCTCTGCCCTCGTCGACGGCCACAGCGACATCATCCCGGAG
ACCGGCAAGCGGCGCCCCAAGTTCCGTATCGAGCTGCCCGGTAACCCCATTCTCG
GTGACGGTAAATCCGACAATCAGAACCACGCTATCGTCTTCTACCGCGGCGAGTAC
CTCCAGCTTATCGACGCCAACCAGGACAACTACCTCGAGGAGTGCTTGAAGATCC
GTAACGTGCTCGCCGAGTTTGAGGAGTACGACGTCTCCAGCCAGAGCCCGTACGC
GCAGTGGAGTGTCAAGGAGTTCAAGCGCTCTCCGGTCGCCATCGTCGGTGCACGC
GAGTACATCTTCTCAGAGCACATCGGTATCCTCGGTGATCTGGCGGCTGGCAAGG
AACAGACGTTCGGTACGCTCACGGCACGCAACAACGCCTTCCTTGGCGGCAAGCT
GCACTACGGTCACCCCGATTTCCTCAACGCCCTCTACATGAACACGCGCGGTGGT
GTCTCCAAGGCGCAGAAGGGTCTCCATCTCAACGAGGATATCTACGCCGGTATGA
ACGCGGTCGGTCGCGGTGGACGCATTAAGCACAGCGAGTACTATCAGTGCGGCAA
GGGTCGTGACCTCGGTTTCGGCACCATCTTGAACTTCCAGACCAAGATCGGTACG
GGTATGGGCGAGCAGATCCTCTCGCGCGAGTACTACTATCTCGGAACACAACTGC
CCATCGATCGCTTCCTCACGTTCTACTACGCGCACCCGGGTTTCCAGATCAACAAC
ATGCTGGTCATCCTCTCCGTGCAGGTCTTCATCGTTACCAgtacgttcaatgcatattgttagcct
gacaacgtctgacgaatttccagTGGTCTTCCTCGGTACCTTGAAGTCTTCGGTCACGATCTG
CAAGTACACGTCCAGCGGTCAGTACATCGGTGGTCAATCCGGTTGCTACAACCTCG
TCCCGGTCTTCCAGTGGATCGAGCGCTGCATCATCAGCATCTTCTTGGTGTTCATG
ATCGCTTTCATGCCGCTCTTCCTGCAAGgtaagagcttgtcaacctgctcaaggggcttgcgctgatcat
catctcagAACTCGTCGAGCGCGGTACCTGGAGTGCCATCTGGCGTCTGCTCAAGCAG
TTTATGTCGCTGTCGCCTGTCTTCGAGGTGTTCTCCACCCAGATTCAGACGCACTC
CGTGTTGAGCAACTTGACGTTCGGTGGTGCGCGTTACATCGCTACCGGTCGTGGG
TTCGCCACCAGTCGTATCAGCTTCAGCATCTTGTTCTCGCGTTTCGCAGGCCCGAG
TATCTACCTCGGCATGCGCACGCTCATTATGCTGCTCTACGTGACGTTGACGATCT
GGACGCCATGGGTCATTTACTTCTGGGTTTCCATTCTCTCGCTCTGCATCGCGCCG
TTCTTGTTCAACCCGCATCAATTCGTATTCTCGGACTTCCTCATCGACTACAGgtacgt
cggacgagcgctgttccgcgacgtaagctgaccggttatacagGGAATACCTGCGGTGGATGTCGCGT
GGCAACTCGCGCTCGCACAACAACTCCTGGATTGGGTACTGCCGGTTGTCCCGCA
CGATGATCACTGGGTACAAGAAGAAGAAGCTGGGCCACCCGTCGGAGAAGCTTTC
CGGCGACGTTCCTCGTGCAGGCTGGCGCGCCGTCTTGTTCTCGGAGATCATCTTC
CCGGCGTGCATGGCCATCCTCTTCATCATCGCGTACATGTTCGTCAAGTCGTTCCC
TCTCGACGGCAAGCAGCCTCCCTCCGGCCTCGTTCGCATCGCCGTCGTGTCTATC
GGCCCCATCGTGTGGAACGCCGCCATCCTGTTGACGCTCTTCCTTGTGTCGTTGTT
CCTCGGCCCCATGCTCGACCCGGTCTTCCCCCTCTTCGGTTCCGTTATGGCCTTCA
TCGCGCATTTCCTTGGCACAATCGGAATGATTGGGTTCTTCGAGTTCCTGgtatgtgccc
atacctttcattcgacttcaactatctaacagattcatagTGGTTCCTCGAGTCCTGGGAGGCGTCGCAT
GCCGTGCTGGGTCTCATCGCCGTCATCTCCATCCAGCGCGCCATTCACAAGATCCT
TATCGCCGTTTTCCTCAGTCGCGAGTTCAAGCACGACGAGACGAACAGGGCCTGG
TGGACTGGTCGCTGGTATGGCCGTGGCCTCGGCACGCACGCCATGTCGCAGCCG
GCGCGTGAGTTCGTCGTCAAGATCATCGAGTTGTCGCTTTGGAGCTCGGATCTCAT
ACTCGGCCACATCCTGCTGTTCATGCTTACTCCGGCCGTCCTCATCCCGTACTTCG
ACCGTTTGCACGCCATGATGCTCTgtacgtcgtgtctcattgtctgtgttggtcatactcttaccctctcttagTC
TGGCTGCGTCCCTCGAAGCAAATCCGCGCGCCTCTGTACTCGATCAAGCAGAAGA
GGCAAAGACGCTGGATTgtcagtgttcagtgccttattctatcagctcttactaacgtcttcatagATCATGAA
GTACGGTACTGTATACGTTACCGTCATCGCGATCTTCGTCGCGCTCATCGCGCTTC
gtgagtttccttgctatttttcgtacctgagcgtcgctgacccctttcccagCCCTCGTATTCCGACACACTCTAA
AGGTCGAGTGCTCCCTTTGCGACAGCTTGTAATATCGGACTCGTATATATCTAGACT
TCTCCGCACCATGTGTAGCTGACGCTTGGGTATACTTCGCGGTGCCGAGCTAATTG
TCGACGGACATTCTCCATCGTTGAGTGCAGCGACGTCGGGTGGTTTACGACACGG
ACACTTTTCATTGTACCCTCTACGAATGCAAGAACTCTCTTACGACCAGTACCTATG
TGCTAAGCCGTCGCCTGTTCAGGATCATACATACATACGTTTCTAGATACCTTACAG
TTAGGCCTATTCAGGGAGAGTCTGCATAAAA
SEQ ID NO: 12
translation of SEQ ID NO: 15
amino acid
S. commune
MPRPGGTSAEGGYASSPSMETTPSDPFGTANGAPRRYYDNDSEEYGPGRRDTYASD
SSNQGLTDPGYYDQNGAYDPYPTGDTDSDGDVYGQRYGPSAESLGTHKFGHSDSST
PTFVDYSASSGGRDSYPAWTAERNIPLSKEEIEDIFLDLTQKFGFQRDSMRNMFDFTM
QLLDSRASRMTPNQALLTLHADYIGGQHANYRKWYFAAQLDLDDAVGQTQNPGLNRL
KSTRGSGKRPRHEKSLNTALERWRQAMNNMSQYDRLRQIALYLLCWGEAAQVRFMP
ECLCFIFKCADDYYRSPECQNRMEPVPEGLYLRTVVKPLYRFVRDQGYEVVEGKFVRR
ERDHDQIIGYDDVNQLFWYPEGIARIVLSDKSRLVDLPPAQRFMKFDRIEWNRVFFKTF
YETRSFTHLVDFNRIWVVHIALYFFYTAYNSPTIYAINGNTPTSLAWSATALGGAVATGI
MILATIAEFSHIPTIWNNTSHLTRRLAFLLVTLGLTCGPTFYVAIAESNGSGGSLALILGIV
QFFISVVATALFTIMPSGRMFGDRVAGKSRKYLASQTFTASYPSLPKHQRFASLLMWFL
IFGCKLTESYFFLTLSFRDPIRVMVGMKIQNCEDKIFGSGLCRNHAAFTLTIMYIMDLVLF
FLDTFLWYVIWNSVFSIARSFVLGLSIWTPWRDIFQRLPKRIYAKLLATGDMEVKYKPKV
LVSQIWNAIIISMYREHLISIEHVQKLLYHQVDTGEAGKRSLRAPPFFVAQGSSGGSGEF
FPPGSEAERRISFFAQSLSTEIPQPIPVDAMPTFTVLTPHYSEKILLSLREIIREEDQNTRV
TLLEYLKQLHPVEWENFVKDTKILAEESAMFNGPSPFGNDEKGQSKMDDLPFYCIGFK
SAAPEYTLRTRIWASLRAQTLYRTVSGMMNYAKAIKLLYRVENPEVVQQFGGNTDKLE
RELERMARRKFKFLVSMQRYSKFNKEEHENAEFLLRAYPDLQIAYLEEEPPRKEGGDP
RIFSALVDGHSDIIPETGKRRPKFRIELPGNPILGDGKSDNQNHAIVFYRGEYLQLIDANQ
DNYLEECLKIRNVLAEFEEYDVSSQSPYAQWSVKEFKRSPVAIVGAREYIFSEHIGILGD
LAAGKEQTFGTLTARNNAFLGGKLHYGHPDFLNALYMNTRGGVSKAQKGLHLNEDIYA
GMNAVGRGGRIKHSEYYQCGKGRDLGFGTILNFQTKIGTGMGEQILSREYYYLGTQLPI
DRFLTFYYAHPGFQINNMLVILSVQVFIVTMVFLGTLKSSVTICKYTSSGQYIGGQSGCY
NLVPVFQWIERCIISIFLVFMIAFMPLFLQELVERGTWSAIWRLLKQFMSLSPVFEVFSTQ
IQTHSVLSNLTFGGARYIATGRGFATSRISFSILFSRFAGPSIYLGMRTLIMLLYVTLTIWT
PWVIYFWVSILSLCIAPFLFNPHQFVFSDFLIDYREYLRWMSRGNSRSHNNSWIGYCRL
SRTMITGYKKKKLGHPSEKLSGDVPRAGWRAVLFSEIIFPACMAILFIIAYMFVKSFPLDG
KQPPSGLVRIAVVSIGPIVWNAAILLTLFLVSLFLGPMLDPVFPLFGSVMAFIAHFLGTIG
MIGFFEFLWFLESWEASHAVLGLIAVISIQRAIHKILIAVFLSREFKHDETNRAWWTGRW
YGRGLGTHAMSQPAREFVVKIIELSLWSSDLILGHILLFMLTPAVLIPYFDRLHAMMLFW
LRPSKQIRAPLYSIKQKRQRRWIIMKYGTVYVTVIAIFVALIALPLVFRHTLKVECSLCDSL
SEQ ID NO: 13
cDNA 1,3-β-D-glucan synthase I of S. commune
strain Lu15634
DNA
S. commune
ATGTCCGGTCCAGGATATGGCAGGAATCCATTCGACAATCCCCCGCCCAACAGAG
GTCCCTATGGCCAGCAGCCAGGTTTCCCGGGGCCCGGCCCTCGGCCTTACGACTC
GGACGCGGACATGAGCCAGACCTATGGCAGCACAACCAGGCTCGCCGGCAGTGC
CGGTTACAGCGACAGAAACGGCAGCTTCGACGGCGACCGCTCCTACGCGCCCTCA
ATTGACTCGCGCGCCAGCGTGCCCAGCATATCGCCCTTCGCAGACCCGGGTATCG
GCTCTAATGAGCCGTATCCCGCTTGGTCGGTCGAACGCCAGATCCCCATGTCCAC
GGAGGAGATTGAGGATATCTTCCTCGACCTCACCCAAAAGTTTGGCTTCCAGCGCG
ACTCCATGCGGAATACGTTCGACTTCATGATGCACCTCCTTGATTCCCGTGCCTCG
CGCATGACGCCCAACCAAGCTCTGCTCACGCTTCACGCCGACTACATTGGTGGCC
AGCACGCCAACTATAGGAAGTGGTATTTCGCCGCTCAGCTCAACCTCGATGACGC
GGTCGGGCAAACCAATAACCCCGGTATCCAGCGCTTGAAGACCATCAAGGGCGCT
ACGAAGACCAAGTCGCTCGACAGCGCACTCAACCGCTGGCGCAATGCGATGAACA
ACATGAGCCAGTACGATCGCCTCCGGCAAATTGCGCTCTATCTCCTCTGCTGGGGA
GAAGCAGGCAACATCCGTCTGGCGCCCGAGTGCTTGTGCTTCATCTTCAAGTGCG
CGGACGACTACTACAGAAGTCCCGAGTGTCAGAACCGGATGGACCCCGTGCCGGA
AGGGCTGTACCTCCAGACGGTCATCAAGCCGCTCTATCGCTTCCTACGTGATCAGG
CGTACGAAGTCGTTGATGGGAAGCAAGTGAAGCGCGAGAAGGACCACGACCAGAT
TATCGGTTATGACGACGTCAACCAGTTATTCTGGTATCCGGAAGGTTTGGCTAAGA
TCGTCATGTCGGACAACACACGACTTGTAGATGTACCTCCGGCGCAGCGGTTCATG
AAGTTCGCCAAGATCGAGTGGAACCGCGTCTTCTTCAAGACGTACTTTGAGAAGCG
CTCTACTGCCCATCTCCTGGTCAACTTCAACCGTATATGGATCCTCCACGTCTCGAT
GTACTTCTTCTACACGGCATTCAACTCTCCACGAGTCTACGCGCCGCACGGCAAAC
TCGACCCCTCCCCTGAGATGACCTGGTCCGCGACTGCCCTTGGAGGCGCTGTGTC
CACCATGATCATGATCCTTGCCACTATCGCGGAGTACACCTACATCCCCACGACAT
GGAACAATGCGTCGCACCTCACCACGCGGCTCATTTTCCTCCTGGTCATCCTCGCG
CTCACTGCTGGACCAACATTCTATATCGCCATGATAGACGGACGCACGGACATCGG
CCAAGTACCACTCATCGTGGCCATAGTGCAGTTCTTCATCTCCGTCGTCGCCACCC
TCGCTTTCGCTACCATCCCTTCTGGTCGCATGTTCGGCGACCGTGTGGCTGGCAA
GTCAAGAAAGCACATGGCATCGCAGACGTTCACAGCGTCGTACCCGTCCATGAAG
CGGTCATCTCGCGTAGCGAGTATCATGCTGTGGCTTTTGGTCTTTGGCTGCAAATA
CGTCGAGTCTTACTTCTTCTTGACGTCCTCCTTCTCCAGCCCGATCGCGGTCATGG
CGCGTACGAAGGTACAGGGCTGCAACGACCGTATCTTCGGCAGCCAGCTGTGCAC
GAATCAGGTCCCGTTCGCGCTGGCAATCATGTACGTGATGGACCTGGTACTGTTCT
TCCTGGACACGTACCTGTGGTACATCATCTGGCTGGTGATCTTCTCGATGGTGCGC
GCGTTCAAGCTTGGTATCTCGATCTGGACGCCCTGGAGCGAGATCTTCACCCGCAT
GCCGAAGCGTATCTACGCGAAGCTGCTGGCGACGGCCGAGATGGAGGTCAAGTAT
AAGCCCAAGGTGCTCGTCTCGCAAATCTGGAACGCGGTCATCATCTCCATGTACCG
GGAGCATCTCTTGTCCATCGAGCACGTCCAGCGCCTGCTATACCACCAGGTTGATG
GTCCAGACGGTCGCCGCACCCTCAGGGCACCGCCGTTCTTCACCAGCCAGCGAAC
TGCGAAGCCAGGCCTGTTCTTCCCTCCTGGTGGCGAGGCTGAGCGCCGTATCTCG
TTCTTTGCCTCATCGCTGACGACCGCGCTCCCTGAGCCTCTGCCGATCGACGCCAT
GCCCACCTTCACCGTGCTCGTTCCCCATTACTCGGAGAAGATTCTGCTCAGTCTGC
GCGAGATTATTCGCGAGGAGGACCAGAACACCCGCGTCACCTTGCTGGAGTACCT
CAAGCAGCTCCACCCTGTCGAATGGGACAACTTCGTCAAGGACACCAAGATCTTGG
CGGAAGAGTCGGGCGACGTCCAGGACGAGAAGCGCGCGCGCACGGACGACTTGC
CGTTCTACTGCATCGGGTTCAAGACCTCGTCACCAGAGTACACCCTGCGTACGCGT
ATCTGGGCTTCACTGCGCGCACAGACGCTGTACCGCACGGTCTCCGGTATGATGA
ACTACTCCAAGGCGATCAAGCTCCTCTATCGCGTCGAGAACCCGGATGTCGTTCAT
GCCTTCGGTGGGAACACGGAACGTCTTGAACGCGAGCTTGAGCGCATGTCTCGCC
GCAAGTTCAAGTTCGTCATCTCGATGCAGCGGTACTCTAAGTTCAACAAGGAGGAG
CAAGAGAACGCCGAATTCCTTCTGCGCGCGTACCCGGATTTGCAGATCGCGTACC
TCGATGAAGAGCCCGGTCCCAGCAAGAGCGACGAGGTTCGGTTGTTTTCGACACT
CATCGATGGACACTCCGAGGTGGATGAGAAGACCGGCCGCCGCAAGCCCAAGTTC
CGCATTGAGCTGCCCGGTAACCCCATCCTCGGTGACGGGAAGTCGGATAACCAGA
ACCACGCCATTGTCTTCTACCGCGGCGAGTACATCCAGGTCATCGACGCTAACCAG
GACAATTACCTGGAAGAGTGTCTCAAGATCCGTAACGTCCTGGGCGAGTTTGAGGA
ATACTCCGTGTCGAGCCAGAGCCCGTACGCACAGTGGGGCCACAAGGAGTTCAAC
AAGTGCCCCGTCGCTATCCTGGGTTCTCGCGAGTACATCTTCTCGGAGAACATCGG
TATCCTCGGTGACATCGCCGCCGGCAAGGAACAGACGTTCGGTACCATTACGGCG
CGTGCGCTTGCGTGGATCGGCGGCAAGCTGCATTACGGTCACCCGGATTTCCTCA
ATGCGACGTTCATGACGACGCGTGGTGGCGTGTCAAAAGCGCAGAAGGGCTTGCA
TCTCAACGAGGATATCTTCGCTGGTATGACCGCCGTGTCCCGCGGAGGGCGCATC
AAGCACATGGAGTACTACCAGTGCGGCAAAGGTCGTGATCTCGGTTTCGGCACGA
TCTTGAACTTCCAGACGAAGATCGGTACTGGTATGGGCGAGCAGCTCCTCTCGCG
CGAGTACTACTACCTGGGCACGCAATTGCCTATCGACCGGTTCTTGACGTTCTACT
ACGCGCACGCTGGTTTCCACGTCAACAACATCCTGGTCATCTACTCCATCCAGGTC
TTCATGGTCACCTTGCTGTACCTGGGCACATTGAACAAGCAGCTGTTCATCTGCAA
GGTCAACTCCAATGGCCAGGTTCTTAGTGGACAAGCTGGGTGCTACAACCTCATCC
CGGTCTTCGAGTGGATTCGCCGGAGTATCATCTCCATCTTCTTGGTGTTCTTCATC
GCCTTCTTGCCTCTATTCTTGCAAGAGCTGTGCGAGCGCGGAACGGGAAAGGCGT
TGCTGCGTCTCGGGAAGCACTTCTTGTCACTGTCGCCCATTTTCGAAGTGTTCTCC
ACCCAGATTTACTCGCAGGCGCTCTTGAACAACATGAGCTTCGGTGGTGCGCGCTA
CATCGCCACAGGTCGTGGTTTCGCGACTAGTCGCATACCCTTCAACATCCTCTACT
CGCGTTTCGCGCCGCCAAGCATCTACATGGGCATGCGTAACCTGCTGCTCCTGCT
GTACGCGACGATGGCCATTTGGATCCCGCACCTGATCTACTTCTGGTTCTCCGTCC
TCTCCCTCTGCATCGCGCCATTCATGTTCAATCCGCATCAATTCTCGTACGCCGACT
TCATCATCGACTACCGGGAGTTCTTGCGCTGGATGTCGCGCGGTAACTCGCGAAC
GAAGGCGAGCAGCTGGTACGGATACTGCCGTCTGTCGCGTACCGCGATTACTGGG
TACAAGAAGAAGAAGCTGGGACACCCGTCGGAGAAGCTGTCGGGCGACGTACCG
CGTGCGCCGTGGAGGAACGTTATCTTCTCGGAGATCCTGTGGCCCATCGGCGCGT
GCATCATCTTCATCGTCGCGTACATGTTCGTCAAGTCGTTCCCCGACGAGCAGGGC
AACGCGCCGCCGAGCCCGCTGGTCCGGATTCTGCTCATCGCGGTTGGCCCTACTG
TGTGGAACGCGGCGGTGCTCATAACGCTGTTCTTCCTGTCGCTCTTCCTGGGCCC
GATGATGGATGGCTGGGTCAAGTTCGGCTCGGTCATGGCGGCCCTTGCGCATGGC
CTGGCGCTTATAGGCATGCTCACGTTCTTTGAGTTCTTCTGGTTCCTTGAGCTCTG
GGATGCCTCGCACGCCGTGCTCGGCGTCATCGCTATCATTGCCGTTCAGCGCGGG
ATCCAGAAGATCCTCATTGCCGTCTTCCTGACGCGTGAGTACAAGCACGACGAGAC
GAACCGCGCGTGGTGGACAGGTAAATGGTATGGACGCGGGCTGGGTACCTCGGC
CATGTCCCAGCCGGCGCGCGAGTTCATCGTGAAGATCGTGGAGATGTCGTTGTGG
ACGTCGGACTTCCTGCTTGCGCACCTGTTGCTCATCATCTTGACGGTGCCGCTACT
GCTGCCGTTCTTCAACTCAATTCATTCGACGATGCTTTTCTGGTTGCGCCCTTCGAA
GCAGATTAGGCAACCTCTGTTCTCCACCAAGCAGAAGCGGCAACGGCGATGGATT
GTCATGAAGTATACCGTGGTATATCTCGTGGTGGTGGCTTTCCTCGTCGCGCTCAT
CGCTCTGCCCGCCCTCTTCCGCGAGAGCATCCACTTCAACTGCGAGATCTGCCAG
AGTATATAG
SEQ ID NO: 14
polypeptide sequence 1,3-β-D-glucan synthase I
of S. commune strain Lu15634
amino acid
S. commune
MSGPGYGRNPFDNPPPNRGPYGQQPGFPGPGPRPYDSDADMSQTYGSTTRLAGSA
GYSDRNGSFDGDRSYAPSIDSRASVPSISPFADPGIGSNEPYPAWSVERQIPMSTEEIE
DIFLDLTQKFGFQRDSMRNTFDFMMHLLDSRASRMTPNQALLTLHADYIGGQHANYRK
WYFAAQLNLDDAVGQTNNPGIQRLKTIKGATKTKSLDSALNRWRNAMNNMSQYDRLR
QIALYLLCWGEAGNIRLAPECLCFIFKCADDYYRSPECQNRMDPVPEGLYLQTVIKPLY
RFLRDQAYEVVDGKQVKREKDHDQIIGYDDVNQLFWYPEGLAKIVMSDNTRLVDVPPA
QRFMKFAKIEWNRVFFKTYFEKRSTAHLLVNFNRIWILHVSMYFFYTAFNSPRVYAPHG
KLDPSPEMTWSATALGGAVSTMIMILATIAEYTYIPTTWNNASHLTTRLIFLLVILALTAGP
TFYIAMIDGRTDIGQVPLIVAIVQFFISVVATLAFATIPSGRMFGDRVAGKSRKHMASQTF
TASYPSMKRSSRVASIMLWLLVFGCKYVESYFFLTSSFSSPIAVMARTKVQGCNDRIFG
SQLCTNQVPFALAIMYVMDLVLFFLDTYLWYIIWLVIFSMVRAFKLGISIWTPWSEIFTRM
PKRIYAKLLATAEMEVKYKPKVLVSQIWNAVIISMYREHLLSIEHVQRLLYHQVDGPDGR
RTLRAPPFFTSQRTAKPGLFFPPGGEAERRISFFASSLTTALPEPLPIDAMPTFTVLVPH
YSEKILLSLREIIREEDQNTRVTLLEYLKQLHPVEWDNFVKDTKILAEESGDVQDEKRAR
TDDLPFYCIGFKTSSPEYTLRTRIWASLRAQTLYRTVSGMMNYSKAIKLLYRVENPDVV
HAFGGNTERLERELERMSRRKFKFVISMQRYSKFNKEEQENAEFLLRAYPDLQIAYLDE
EPGPSKSDEVRLFSTLIDGHSEVDEKTGRRKPKFRIELPGNPILGDGKSDNQNHAIVFY
RGEYIQVIDANQDNYLEECLKIRNVLGEFEEYSVSSQSPYAQWGHKEFNKCPVAILGSR
EYIFSENIGILGDIAAGKEQTFGTITARALAWIGGKLHYGHPDFLNATFMTTRGGVSKAQ
KGLHLNEDIFAGMTAVSRGGRIKHMEYYQCGKGRDLGFGTILNFQTKIGTGMGEQLLS
REYYYLGTQLPIDRFLTFYYAHAGFHVNNILVIYSIQVFMVTLLYLGTLNKQLFICKVNSN
GQVLSGQAGCYNLIPVFEWIRRSIISIFLVFFIAFLPLFLQELCERGTGKALLRLGKHFLSL
SPIFEVFSTQIYSQALLNNMSFGGARYIATGRGFATSRIPFNILYSRFAPPSIYMGMRNLL
LLLYATMAIWIPHLIYFWFSVLSLCIAPFMFNPHQFSYADFIIDYREFLRWMSRGNSRTK
ASSWYGYCRLSRTAITGYKKKKLGHPSEKLSGDVPRAPWRNVIFSEILWPIGACIIFIVAY
MFVKSFPDEQGNAPPSPLVRILLIAVGPTVWNAAVLITLFFLSLFLGPMMDGWVKFGSV
MAALAHGLALIGMLTFFEFFWFLELWDASHAVLGVIAIIAVQRGIQKILIAVFLTREYKHDE
TNRAWWTGKWYGRGLGTSAMSQPAREFIVKIVEMSLWTSDFLLAHLLLIILTVPLLLPFF
NSIHSTMLFWLRPSKQIRQPLFSTKQKRQRRWIVMKYTVVYLVVVAFLVALIALPALFRE
SIHFNCEICQSI
SEQ ID NO: 15
cDNA 1,3-β-D-glucan synthase II of S. commune
strain Lu15634
DNA
S. commune
ATGCCGAGGCCGGGCGGCACCAGCGCAGAAGGCGGCTACGCATCATCGCCGTCG
ATGGAGACGACCCCCAGCGATCCCTTCGGAACCGCGAACGGCGCGCCCCGCCGC
TACTACGACAATGATTCTGAGGAGTACGGACCTGGCCGTAGAGACACCTACGCGT
CCGACAGCAGTAATCAGGGCCTCACGGACCCGGGCTACTACGACCAGAATGGCGC
CTATGATCCCTATCCGACCGGGGACACCGATTCCGACGGCGACGTCTACGGCCAG
CGATATGGACCCTCAGCAGAGTCGCTTGGCACCCACAAGTTCGGCCATTCCGATTC
ATCCACGCCGACTTTTGTCGACTACAGCGCATCCTCCGGCGGGAGGGATTCGTAC
CCTGCATGGACTGCCGAACGCAACATCCCGCTGTCCAAGGAGGAGATCGAGGACA
TCTTCCTCGATTTGACGCAGAAGTTTGGCTTTCAGCGGGATTCCATGCGGAATATG
TTCGACTTCACCATGCAGCTGCTTGACAGCCGAGCGTCTCGTATGACCCCCAACCA
GGCGCTCCTCACCCTCCACGCCGACTACATTGGTGGCCAGCATGCGAACTACCGG
AAGTGGTACTTCGCGGCGCAGCTCGACCTTGACGACGCCGTGGGACAAACTCAGA
ATCCGGGTCTCAACCGCCTCAAGTCCACTCGCGGATCGGGCAAGCGACCACGCCA
TGAAAAGTCGCTGAACACGGCATTGGAGCGCTGGCGGCAAGCCATGAACAACATG
TCGCAGTATGACCGCTTACGCCAGATCGCGCTCTACCTGCTCTGCTGGGGCGAAG
CGGCGCAAGTGCGATTCATGCCCGAGTGCTTGTGCTTCATCTTCAAGTGCGCCGA
CGACTACTATCGTTCGCCGGAGTGCCAGAACAGGATGGAGCCGGTACCGGAGGGT
CTCTACCTGAGGACGGTCGTAAAGCCGCTCTACAGATTTGTCCGGGATCAAGGCTA
TGAGGTGGTGGAGGGAAAATTCGTACGGCGGGAACGGGATCACGACCAAATCATT
GGTTACGATGACGTGAATCAGCTGTTCTGGTACCCGGAGGGAATTGCCCGTATCGT
CCTGTCGGACAAGAGTCGTCTAGTCGACCTCCCCCCAGCACAGCGCTTCATGAAG
TTCGACCGTATCGAGTGGAATCGCGTCTTCTTCAAGACGTTTTACGAGACTCGATC
CTTCACGCATCTTTTGGTCGACTTCAACCGTATCTGGGTCGTGCACATCGCTCTCTA
CTTCTTCTACACTGCATACAACTCCCCCACGATCTACGCCATCAACGGCAACACAC
CGACGTCTCTGGCTTGGAGCGCGACTGCGCTCGGCGGTGCGGTAGCGACAGGTA
TCATGATCCTCGCCACGATCGCCGAGTTCTCGCACATCCCCACGACATGGAACAAC
ACCTCGCATCTGACTCGCCGCCTCGCCTTCCTCCTCGTCACGCTCGGCCTCACAT
GTGGTCCGACGTTCTACGTCGCGATTGCAGAGAGCAACGGGAGCGGCGGCTCTTT
GGCCTTGATTCTCGGTATCGTCCAGTTCTTCATCTCCGTCGTGGCAACTGCGCTCT
TCACTATCATGCCTTCTGGTCGTATGTTCGGCGACCGTGTCGCAGGCAAGAGTCGC
AAGTATCTCGCCAGCCAGACGTTCACGGCCAGCTACCCGTCGTTGCCCAAGCACC
AGCGGTTCGCCTCACTCCTGATGTGGTTCCTCATCTTCGGGTGCAAGTTGACGGAG
AGTTACTTCTTTCTGACGCTGTCCTTCCGCGACCCTATCCGCGTCATGGTCGGCAT
GAAGATCCAGAACTGCGAGGACAAGATTTTCGGCAGCGGCCTTTGCAGGAATCAC
GCAGCATTCACCCTCACGATCATGTACATCATGGACCTCGTCTTGTTCTTCCTCGAC
ACCTTCCTTTGGTATGTCATCTGGAACTCGGTTTTCAGTATCGCACGCTCTTTCGTA
CTCGGCCTTTCGATCTGGACACCGTGGAGAGACATCTTCCAGCGTCTGCCGAAGC
GGATCTACGCGAAGCTTCTGGCGACTGGCGACATGGAGGTCAAGTACAAGCCCAA
GGTCTTGGTCTCGCAAATCTGGAACGCCATCATCATCTCCATGTACCGCGAGCACT
TGCTCTCTATTGAGCACGTCCAGAAGCTCCTGTACCACCAAGTGGACACTGGCGAA
GCCGGCAAGCGGAGTCTTCGCGCGCCTCCGTTCTTCGTCGCGCAGGGCAGCAGC
GGTGGCTCGGGCGAGTTCTTCCCGCCTGGCAGCGAGGCCGAGCGTCGTATCTCTT
TCTTCGCGCAGTCGCTTTCTACGGAGATTCCTCAGCCCATCCCGGTCGACGCCATG
CCGACGTTCACGGTGCTTACGCCTCACTACAGCGAGAAGATCCTTCTCTCTCTCCG
TGAAATTATCCGCGAGGAGGACCAGAACACTCGCGTTACGTTGCTCGAGTACCTGA
AGCAGCTGCATCCGGTCGAGTGGGAGAATTTCGTCAAGGACACTAAAATTTTGGCC
GAGGAGTCCGCTATGTTTAACGGTCCGAGTCCTTTCGGCAACGACGAGAAGGGTC
AGTCCAAGATGGACGATCTACCGTTCTACTGCATCGGTTTCAAGAGCGCCGCGCC
CGAGTACACCCTCCGCACCCGTATCTGGGCGTCCCTGCGCGCGCAGACGCTGTAC
CGCACGGTCTCCGGCATGATGAACTATGCGAAGGCGATCAAGCTGCTCTACCGCG
TTGAGAACCCGGAGGTCGTACAACAGTTCGGCGGCAACACGGACAAGCTCGAGCG
CGAGTTGGAGCGGATGGCGCGACGGAAGTTCAAGTTCCTCGTGTCCATGCAGCGC
TACTCGAAGTTCAACAAGGAGGAGCACGAGAACGCCGAGTTCTTGCTCCGCGCGT
ACCCGGACTTGCAGATCGCGTACCTCGAGGAAGAGCCCCCTCGCAAGGAGGGCG
GCGATCCACGCATCTTCTCTGCCCTCGTCGACGGCCACAGCGACATCATCCCGGA
GACCGGCAAGCGGCGCCCCAAGTTCCGTATCGAGCTGCCCGGTAACCCCATTCTC
GGTGACGGTAAATCCGACAATCAGAACCACGCTATCGTCTTCTACCGCGGCGAGTA
CCTCCAGCTTATCGACGCCAACCAGGACAACTACCTCGAGGAGTGCTTGAAGATCC
GTAACGTGCTCGCCGAGTTTGAGGAGTACGACGTCTCCAGCCAGAGCCCGTACGC
GCAGTGGAGTGTCAAGGAGTTCAAGCGCTCTCCGGTCGCCATCGTCGGTGCACGC
GAGTACATCTTCTCAGAGCACATCGGTATCCTCGGTGATCTGGCGGCTGGCAAGG
AACAGACGTTCGGTACGCTCACGGCACGCAACAACGCCTTCCTTGGCGGCAAGCT
GCACTACGGTCACCCCGATTTCCTCAACGCCCTCTACATGAACACGCGCGGTGGT
GTCTCCAAGGCGCAGAAGGGTCTCCATCTCAACGAGGATATCTACGCCGGTATGA
ACGCGGTCGGTCGCGGTGGACGCATTAAGCACAGCGAGTACTATCAGTGCGGCAA
GGGTCGTGACCTCGGTTTCGGCACCATCTTGAACTTCCAGACCAAGATCGGTACG
GGTATGGGCGAGCAGATCCTCTCGCGCGAGTACTACTATCTCGGAACACAACTGC
CCATCGATCGCTTCCTCACGTTCTACTACGCGCACCCGGGTTTCCAGATCAACAAC
ATGCTGGTCATCCTCTCCGTGCAGGTCTTCATCGTTACCATGGTCTTCCTCGGTAC
CTTGAAGTCTTCGGTCACGATCTGCAAGTACACGTCCAGCGGTCAGTACATCGGTG
GTCAATCCGGTTGCTACAACCTCGTCCCGGTCTTCCAGTGGATCGAGCGCTGCATC
ATCAGCATCTTCTTGGTGTTCATGATCGCTTTCATGCCGCTCTTCCTGCAAGAACTC
GTCGAGCGCGGTACCTGGAGTGCCATCTGGCGTCTGCTCAAGCAGTTTATGTCGC
TGTCGCCTGTCTTCGAGGTGTTCTCCACCCAGATTCAGACGCACTCCGTGTTGAGC
AACTTGACGTTCGGTGGTGCGCGTTACATCGCTACCGGTCGTGGGTTCGCCACCA
GTCGTATCAGCTTCAGCATCTTGTTCTCGCGTTTCGCAGGCCCGAGTATCTACCTC
GGCATGCGCACGCTCATTATGCTGCTCTACGTGACGTTGACGATCTGGACGCCAT
GGGTCATTTACTTCTGGGTTTCCATTCTCTCGCTCTGCATCGCGCCGTTCTTGTTCA
ACCCGCATCAATTCGTATTCTCGGACTTCCTCATCGACTACAGGGAATACCTGCGG
TGGATGTCGCGTGGCAACTCGCGCTCGCACAACAACTCCTGGATTGGGTACTGCC
GGTTGTCCCGCACGATGATCACTGGGTACAAGAAGAAGAAGCTGGGCCACCCGTC
GGAGAAGCTTTCCGGCGACGTTCCTCGTGCAGGCTGGCGCGCCGTCTTGTTCTCG
GAGATCATCTTCCCGGCGTGCATGGCCATCCTCTTCATCATCGCGTACATGTTCGT
CAAGTCGTTCCCTCTCGACGGCAAGCAGCCTCCCTCCGGCCTCGTTCGCATCGCC
GTCGTGTCTATCGGCCCCATCGTGTGGAACGCCGCCATCCTGTTGACGCTCTTCCT
TGTGTCGTTGTTCCTCGGCCCCATGCTCGACCCGGTCTTCCCCCTCTTCGGTTCCG
TTATGGCCTTCATCGCGCATTTCCTTGGCACAATCGGAATGATTGGGTTCTTCGAGT
TCCTGTGGTTCCTCGAGTCCTGGGAGGCGTCGCATGCCGTGCTGGGTCTCATCGC
CGTCATCTCCATCCAGCGCGCCATTCACAAGATCCTTATCGCCGTTTTCCTCAGTC
GCGAGTTCAAGCACGACGAGACGAACAGGGCCTGGTGGACTGGTCGCTGGTATG
GCCGTGGCCTCGGCACGCACGCCATGTCGCAGCCGGCGCGTGAGTTCGTCGTCA
AGATCATCGAGTTGTCGCTTTGGAGCTCGGATCTCATACTCGGCCACATCCTGCTG
TTCATGCTTACTCCGGCCGTCCTCATCCCGTACTTCGACCGTTTGCACGCCATGAT
GCTCTTCTGGCTGCGTCCCTCGAAGCAAATCCGCGCGCCTCTGTACTCGATCAAG
CAGAAGAGGCAAAGACGCTGGATTATCATGAAGTACGGTACTGTATACGTTACCGT
CATCGCGATCTTCGTCGCGCTCATCGCGCTTCCCCTCGTATTCCGACACACTCTAA
AGGTCGAGTGCTCCCTTTGCGACAGCTTGTAA
SEQ ID NO: 16
polypeptide sequence 1,3-β-D-glucan synthase II
of S. commune strain Lu15634
amino acid
S. commune
MPRPGGTSAEGGYASSPSMETTPSDPFGTANGAPRRYYDNDSEEYGPGRRDTYASD
SSNQGLTDPGYYDQNGAYDPYPTGDTDSDGDVYGQRYGPSAESLGTHKFGHSDSST
PTFVDYSASSGGRDSYPAWTAERNIPLSKEEIEDIFLDLTQKFGFQRDSMRNMFDFTM
QLLDSRASRMTPNQALLTLHADYIGGQHANYRKWYFAAQLDLDDAVGQTQNPGLNRL
KSTRGSGKRPRHEKSLNTALERWRQAMNNMSQYDRLRQIALYLLCWGEAAQVRFMP
ECLCFIFKCADDYYRSPECQNRMEPVPEGLYLRTVVKPLYRFVRDQGYEVVEGKFVRR
ERDHDQIIGYDDVNQLFWYPEGIARIVLSDKSRLVDLPPAQRFMKFDRIEWNRVFFKTF
YETRSFTHLLVDFNRIWVVHIALYFFYTAYNSPTIYAINGNTPTSLAWSATALGGAVATGI
MILATIAEFSHIPTTWNNTSHLTRRLAFLLVTLGLTCGPTFYVAIAESNGSGGSLALILGIV
QFFISVVATALFTIMPSGRMFGDRVAGKSRKYLASQTFTASYPSLPKHQRFASLLMWFL
IFGCKLTESYFFLTLSFRDPIRVMVGMKIQNCEDKIFGSGLCRNHAAFTLTIMYIMDLVLF
FLDTFLWYVIWNSVFSIARSFVLGLSIWTPWRDIFQRLPKRIYAKLLATGDMEVKYKPKV
LVSQIWNAIIISMYREHLLSIEHVQKLLYHQVDTGEAGKRSLRAPPFFVAQGSSGGSGEF
FPPGSEAERRISFFAQSLSTEIPQPIPVDAMPTFTVLTPHYSEKILLSLREIIREEDQNTRV
TLLEYLKQLHPVEWENFVKDTKILAEESAMFNGPSPFGNDEKGQSKMDDLPFYCIGFK
SAAPEYTLRTRIWASLRAQTLYRTVSGMMNYAKAIKLLYRVENPEVVQQFGGNTDKLE
RELERMARRKFKFLVSMQRYSKFNKEEHENAEFLLRAYPDLQIAYLEEEPPRKEGGDP
RIFSALVDGHSDIIPETGKRRPKFRIELPGNPILGDGKSDNQNHAIVFYRGEYLQLIDANQ
DNYLEECLKIRNVLAEFEEYDVSSQSPYAQWSVKEFKRSPVAIVGAREYIFSEHIGILGD
LAAGKEQTFGTLTARNNAFLGGKLHYGHPDFLNALYMNTRGGVSKAQKGLHLNEDIYA
GMNAVGRGGRIKHSEYYQCGKGRDLGFGTILNFQTKIGTGMGEQILSREYYYLGTQLPI
DRFLTFYYAHPGFQINNMLVILSVQVFIVTMVFLGTLKSSVTICKYTSSGQYIGGQSGCY
NLVPVFQWIERCIISIFLVFMIAFMPLFLQELVERGTWSAIWRLLKQFMSLSPVFEVFSTQ
IQTHSVLSNLTFGGARYIATGRGFATSRISFSILFSRFAGPSIYLGMRTLIMLLYVTLTIWT
PWVIYFWVSILSLCIAPFLFNPHQFVFSDFLIDYREYLRWMSRGNSRSHNNSWIGYCRL
SRTMITGYKKKKLGHPSEKLSGDVPRAGWRAVLFSEIIFPACMAILFIIAYMFVKSFPLDG
KQPPSGLVRIAVVSIGPIVWNAAILLTLFLVSLFLGPMLDPVFPLFGSVMAFIAHFLGTIG
MIGFFEFLWFLESWEASHAVLGLIAVISIQRAIHKILIAVFLSREFKHDETNRAWWTGRW
YGRGLGTHAMSQPAREFVVKIIELSLWSSDLILGHILLFMLTPAVLIPYFDRLHAMMLFW
LRPSKQIRAPLYSIKQKRQRRWIIMKYGTVYVTVIAIFVALIALPLVFRHTLKVECSLCDSL
SEQ ID NO: 17
tef1 promoter
DNA
S. commune
ATCGCCATTGTAAGCCGCAGACGGGCACGCTTCCAACCCCCATCGATGGGCGCTC
GATGTCCATCTCATCGGCGACTCATCATTGTATCTCGCGCAGTCCCATCCCTCGCC
GCTCGCCTGTAGTTTATGCTATTTATCTTTGCACCAGTCGTTGTATTACTCCCTCGT
CGTGTAGAAAGTACCAGATAAAATGCATGTAATCCTAATGAAATTTGCACGACACGA
AGATCCGGCAGGGTTGTGGGCAAGGGGCAGCGGGAACGAATGGATGGCGGGGTA
CAGCGAGTACCCGGCAGTGCCACAGTCAGTGTCACACACGTGACTGATTGTCCATT
AGCGTGACCGATAACATCGATCAAAAATTTTATTTCAGAGGACGATAAATAAGGGCC
GACGGTGCGCGTCCGTCTTTCTCTCAACCCTCATCTTCCTCTCGTCTCTCACTCTTC
CCCCCTCCACCACTACCAAGTAAGTTCAAACTTCCTCTCATCGCCTTTGCACACATC
GCCTACGCCCCATCTCTCTCCATCTGCCTCGCGAACGGCGCCCCCATCGTCGCTT
TCCCGCGCGAGATCTTGTGCGATCTAGTTTACTGACAATCTCACCTAGAAAACATCA
AA
SEQ ID NO: 18
tef1 terminator
DNA
S. commune
ATCCAAGTCCGGTGGCAAGGTCACCAAGTCCGCCGAGAAGGCCGCCAAGAAGAAG
TAAATGTAGATGTACATATGTATTTTCTCATTCCGTTTCCTTCCTCTTGTTGTTGTTTC
ACTGGTCCTCTCGTGCTCGCTCGCATCGCATACAGCCATTGTTGTCACCACTATAA
CTTCACGCATTCTGTATTTCATGCCAGGCGACGGGGTGTTCCTGCCAGGCCTGTCG
CTTGTTGTAACGCTAATGAAAAGTCACGAGTAGTGGACGAACGACGATGTATTTCTA
TGTGCTGTAGCGATTATCCATTTCGAGTTCGCCATCGAGCTCTCTTCAAACCTAGGT
GCGACGTTGTGAATGCAGTAGCAAGTGCAGAGTATTGCAGACTCGTCCATTGATGA
TAACTTCAAGCTACGTCAGAGCCAGATGCTACTGAACCCGGGCC
SEQ ID NO: 19
Ura_forw (NotI) primer
DNA
artificial
ATAAGAATGCGGCCGCTCCAGCTCGACCTTGCGCCG
SEQ ID NO: 20
Ura_rev (XbaI) primer
DNA
artificial
CTAGTCTAGAGGATCCGACGTGGAGGAGCC
SEQ ID NO. 21
TefP_forw (XbaI) primer
DNA
artificial
CTAGTCTAGAATCGCCATTGTAAGCCGCAG
SEQ ID NO: 22
TefP_rev (SpeI) primer
DNA
artificial
CTAGACTAGTTTTGATGTTTTCTAGGTGAG
SEQ ID NO: 23
TefT_forw (SalI) primer
DNA
artificial
ACGCGTCGACCAAGTCCGGTGGCAAGGTCA
SEQ ID NO: 24
TefT_rev (SalI) primer
DNA
artificial
CCGACGTCGACGGGTTCAGTAGCATCTGGCT
SEQ ID NO: 25
TefT_forw (EcoRV) primer
DNA
artificial
CATGGTGATATCCAAGTCCGGTGGCAAGGTCA
SEQ ID NO: 26
TefT_rev (ApaI) primer
DNA
artificial
CCGTATGGGCCCGGGTTCAGTAGCATCTGGCT
SEQ ID NO: 27
GS1_forw (SpeI) primer
DNA
artificial
CTAGACTAGTCCCGTCCCTCAAGGCCGTTC
SEQ ID NO: 28
GS1_rev (SalI) primer
DNA
artificial
AATGGCCGACGTCGACATGGTATATGCAATGCTATG
SEQ ID NO: 29
Fusion TefP_GS1_forw (XbaI) primer
DNA
artificial
CTAGTCTAGAATCGCCATTGTAAGCCGCAG
SEQ ID NO: 30
Fusion TefP_GS1_rev (SalI) primer
DNA
artificial
AATGGCCGACGTCGACATGGTATATGCAATGCTATG
SEQ ID NO: 31
GS2_forw (SpeI) primer
DNA
artificial
CTAGACTAGTCTGTCCAAAGAAGAGATCGA
SEQ ID NO: 32
GS2_rev (EcoRV) primer
DNA
artificial
TACATGCGATATCTTTTATGCAGACTCTCCCTG
SEQ ID NO: 33
ura gene
DNA
S. commune
TCCAGCTCGACCTTGCGCCGCTTGGAGTAACGTTCAGCGTCTTCGTCGTCCTCGTC
GCGCTCGTGTACGATGATGGGCTCAGCCATGGCAGGTATACAAGCTCAGAGTCAA
TGGGGGACGAGGTCTCAAGCCGTGAAAGTCGTCGTCGAACAACGTCAAGTTCGAG
ACGGACCAGAGTTGGATTTCGTGATTAGATCTACGCTCGATCACAGAATGATCAAA
GAACAAAGCTTGCCAAAAGGGGATCTCCCATCAACTTCAACTTGCCCCAAACCATC
ATGACCGCCGCTCATAAGCTCACATACGGTCAGCGCGCTGCAAGGTTCACCAATC
CCGCGGCGAAAGCCCTGCTGGAAACCATGGAGCGCAAGAAGAGCAATCTATCCGT
CAGCGTCGACGTCGTAAAATCCGCCGATCTGCTCGCTATTGTCGATACCGTCGGG
CCCTATATCTGTCTGATAAAGGCATTGCACTGTCGCTTGCGGTCTTGGGATGCTGC
TTATACTCTATGAAGACCCATGTGGATGTTGTCGAAGACTTCGACTCGTCGCTCGT
CACCAAGCTTCAGGCTCTGGCCGAGAAGCATGATTTCCTCATCTTTGAGGACAGAA
AATTCGCCGACATAGGTCTGTCCGTCGAATCTCTATCGATGTCAACTCTGATGACTT
GCACAGGCAACACCGTCGCTCTGCAGTACTCTAGTGGCGTGCACAAAATTGCCAG
CTGGTCGCACATCACGAACGCACACCCTGTTCCAGGACCGTCAATCATCAGTGGC
CTCGCATCGGTAGGACAACCCCTCGGTCGCGGACTCCTCCTGCTCGCAGAGATGA
GCACGAAGGGCTCACTTGCGACAGGCGCGTACACTGAAGCCGCCGTCCAGATGG
CAAGGGAGAACCGCGGCTTCGTCATCGGGTTCATCGCCCAACGGCGGATGGATG
GTATTGGCGCGCCTCCAGGGGTGAATGTCGAGGACGAGGATTTTCTTGTCTTGACA
CCAGGTGTCGGACTCGATGTGAAGGGCGATGGGATGGGGCAGCAATACAGGACG
CCGAAGCAAGTGGTACAGGAAGATGGGTGCGATGTAATCATCGTGGGTCGCGGGA
TTTATGGCAAGGACCCATCGAAGGTGGAAGAGATACGGAGGCAGGCAGAGCGTTA
CCAGGCTGCAGGATGGGCGGCGTACATTGAGAGGGTCAACGCCTTGGTATAGCTA
ATCTGATCGGTGTTGTCTTGTTAAGCGTCAGGCTCAATGGAACGCTTTGGACGAGC
GGAGAGTAACTTGAATTAGCAGTGTATACTTCGGGCAAATCAATCGTGATAAATACA
AGAGCACGCTCACGCACGTCCAATCTCCCTCAAAATCTCCATCTTTCTCGCCTCATT
CACCTTCCTGAACCCAGCCGGCGACATCTCGAACAGACCATGCCCACCCGACAGC
GCACGCAGCCTATTCGAGTAGTCCAGCATCCGGCTGAGCGGCGCCACCGCCTGCA
CCGCGCGCTTCATCTTCACGCCCGCCGCCTCCCTCGCCGCAGTGCCGCCAGAGG
GCGACACCCACTCCGGGGGCACGTACACGCCGTCCGCAGGGTACGGCTCCTCCA
CGTCGGATCC
SEQ ID NO: 34
Ura protein
amino acid
S. commune
MTAAHKLTYGQRAARFTNPAAKALLETMERKKSNLSVSVDVVKSADLLAIVDTVGPYIC
LIKTHVDVVEDFDSSLVTKLQALAEKHDFLIFEDRKFADIGNTVALQYSSGVHKIASWSHI
TNAHPVPGPSIISGLASVGQPLGRGLLLLAEMSTKGSLATGAYTEAAVQMARENRGFVI
GFIAQRRMDGIGAPPGVNVEDEDFLVLTPGVGLDVKGDGMGQQYRTPKQVVQEDGC
DVIIVGRGIYGKDPSKVEEIRRQAERYQAAGWAAYIERVNALV

1.-10. (canceled)

11. A genetically modified microorganism capable of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, characterized in that said genetically modified microorganism overexpresses (i) a polynucleotide encoding a polypeptide having 1,3-β-D-glucan synthase-activity, and/or (ii) a polypeptide having 1,3-β-D-glucan synthase-activity, compared to a corresponding non-modified control microorganism of the same strain.

12. Use of a polynucleotide encoding a polypeptide having 1,3-β-D-glucan synthase-activity, or a polypeptide having 1,3-β-D-glucan synthase-activity, or of a genetically modified microorganism according to claim 11 for producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3.

13. A method of producing a polymer consisting of a linear main chain of β-D-(1-3)-glucopyranosyl units having a single β-D-glucopyranosyl unit (1-6) linked to a β-D-glucopyranosyl unit of the linear main chain with an average branching degree of about 0.3, said method comprising the steps of:
(a) introducing (i) a strong promoter upstream of a polynucleotide encoding a polypeptide having 1,3-β-D-glucan synthase-activity thereby increasing the expression of said polynucleotide, or (ii) a polynucleotide encoding a polypeptide having 1,3-β-D-glucan synthase-activity into a microorganism that is able to synthesize said polymer;
(b) culturing said microorganism of (a) in a medium, thereby allowing said microorganism to produce said polymer; and
(c) optionally recovering said polymer from the medium.

14. The genetically modified microorganism according to claim 11, wherein said polymer is selected from the group consisting of schizophyllan, scleroglucan, pendulan, cinerian, laminarin, lentinan and pleuran.

15. The use according to claim 12, wherein said polymer is selected from the group consisting of schizophyllan, scleroglucan, pendulan, cinerian, laminarin, lentinan and pleuran.

16. The method according to claim 13, wherein said polymer is selected from the group consisting of schizophyllan, scleroglucan, pendulan, cinerian, laminarin, lentinan and pleuran.

17. The genetically modified microorganism according to claim 11, wherein said polynucleotide is a 1,3-β-D-glucan synthase gene.

18. The use according to claim 12, wherein said polynucleotide is a 1,3-β-D-glucan synthase gene.

19. The method according to claim 13, wherein said polynucleotide is a 1,3-β-D-glucan synthase gene.

20. The genetically modified microorganism according to claim 11, wherein said polynucleotide comprises a nucleotide sequence being at least 70% identical to the nucleotide sequence of SEQ ID NO: 1, SEQ ID NO: 3, SEQ ID NO: 5, SEQ ID NO: 7, SEQ ID NO: 9, SEQ ID NO: 11, SEQ ID NO: 13, or SEQ ID NO: 15.

21. The use according to claim 12, wherein said polynucleotide comprises a nucleotide sequence being at least 70% identical to the nucleotide sequence of SEQ ID NO: 1, SEQ ID NO: 3, SEQ ID NO: 5, SEQ ID NO: 7, SEQ ID NO: 9, SEQ ID NO: 11, SEQ ID NO: 13, or SEQ ID NO: 15.

22. The method according to claim 13, wherein said polynucleotide comprises a nucleotide sequence being at least 70% identical to the nucleotide sequence of SEQ ID NO: 1, SEQ ID NO: 3, SEQ ID NO: 5, SEQ ID NO: 7, SEQ ID NO: 9, SEQ ID NO: 11, SEQ ID NO: 13, or SEQ ID NO: 15.

23. The genetically modified microorganism according to claim 11, wherein said polypeptide is a 1,3-β-D-glucan synthase.

24. The use according to claim 12, wherein said polypeptide is a 1,3-β-D-glucan synthase.

25. The method according to claim 13, wherein said polypeptide is a 1,3-β-D-glucan synthase.

26. The genetically modified microorganism according to claim 11, wherein said polypeptide comprises an amino acid which is at least 70% identical to the amino acid sequence of SEQ ID NO: 6, SEQ ID NO: 8, SEQ ID NO: 14, or SEQ ID NO: 16.

27. The use according to claim 12, wherein said polypeptide comprises an amino acid which is at least 70% identical to the amino acid sequence of SEQ ID NO: 6, SEQ ID NO: 8, SEQ ID NO: 14, or SEQ ID NO: 16.

28. The method according to claim 13, wherein said polypeptide comprises an amino acid which is at least 70% identical to the amino acid sequence of SEQ ID NO: 6, SEQ ID NO: 8, SEQ ID NO: 14, or SEQ ID NO: 16.

29. The genetically modified microorganism according to claim 11, use according to claim 12, or method according to claim 13, wherein said microorganism is selected from the group consisting of Schizophyllum commune, Sclerotium rolfsii, Sclerotium glucanicum, Sclerotium delphinii, Porodisculus pendulus, Botrytis cinerea, Laminaria sp., Lentinula edoles, and Monilinia fructigena.

30. The use according to claim 12, wherein said microorganism is selected from the group consisting of Schizophyllum commune, Sclerotium rolfsii, Sclerotium glucanicum, Sclerotium delphinii, Porodisculus pendulus, Botrytis cinerea, Laminaria sp., Lentinula edoles, and Monilinia fructigena.

31. The method according to claim 13, wherein said microorganism is selected from the group consisting of Schizophyllum commune, Sclerotium rolfsii, Sclerotium glucanicum, Sclerotium delphinii, Porodisculus pendulus, Botrytis cinerea, Laminaria sp., Lentinula edoles, and Monilinia fructigena.

32. The genetically modified microorganism according to claim 11, wherein said modified microorganism is able to produce at least 1.5 times more of said polymer compared to said non-modified control microorganism.
